(19) United States
(12) Patent Application Publication
(10) Pub. No.: US 2020/0207759 A1
(43) Pub. Date: Jul. 2, 2020

(54) NEW PROPANAMINE DERIVATIVES FOR TREATING PAIN AND PAIN RELATED CONDITIONS

(71) Applicant: ESTEVE PHARMACEUTICALS, S.A., Barcelona (ES)

(72) Inventors: Marina VIRGILI-BERNADO, Barcelona (ES); Carmen ALMANSA-ROSALES, Barcelona (ES); Monica ALONSO-XALMA, Barcelona (ES); Laura OSORIO-PLANES, Tarragona (ES)

(21) Appl. No.: 16/633,595

(22) PCT Filed: Jul. 27, 2018
(86) PCT No.: PCT/EP2018/070418

§ 371 Date: Jan. 24, 2020

(30) Foreign Application Priority Data
Jul. 27, 2017 (EP) 17382502.7

(51) Int. Cl. C07D 471/04; C07D 409/06

(52) U.S. Cl. CPC C07D 471/04; C07D 409/06

(57) ABSTRACT

The present invention relates to new compounds of general formula (I) that show dual activity towards α2δ subunit of voltage-gated calcium channels (VGCC), especially the α2δ-1 subunit, and to the noradrenallne transporter (NET). The invention is also related to the process for the preparation of said compounds as well as to compositions comprising them, and to their use as medicaments.

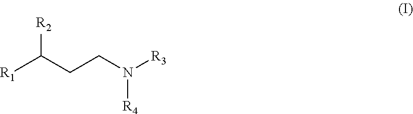

### FIELD OF THE INVENTION

The present invention relates to new compounds that show great affinity and dual activity towards the subunit α2δ of voltage-gated calcium channels (VGCC), especially the α2δ1 subunit of voltage-gated calcium channels and the noradrenaline transporter (NET). The invention is also related to the process for the preparation of said compounds as well as to compositions comprising them, and to their use as medicaments.

### BACKGROUND OF THE INVENTION

The adequate management of pain represents an important challenge, since currently available treatments provide in many cases only modest improvements, leaving many patients unrelieved (Turk, D. C., Wilson, H. D. Cahana, A.; 2011; Lancet. 377; 2226-2235). Pain affects a big portion of the population with an estimated prevalence of 20% and its incidence, particularly in the case of chronic pain, is increasing due to the population ageing. Additionally, pain is clearly correlated to comorbidities, such as depression, anxiety and insomnia, which leads to important productivity losses and socio-economical burden (Goldberg. D. S., McGee, S. J.; 2011; BMC Pubic Heath; 11; 770). Existing pain therapies include non-steroidal anti-inflammatory drugs (NSAIDs), opioid agonists, calcium channel blockers and antidepressants, but they are much less than optimal regarding their safety ratio. All of them show limited efficacy and a range of secondary effects that preclude their use, especially in chronic settings.

Voltage-gated calcium channels (VGCC) are required for many key functions in the body. Different subtypes of voltage-gated calcium channels have been described (Zamponi et al.; Pharmacol. Rev.; 2015; 67; 821-870). The VGCC are assembled through interactions of different subunits, namely α1 (Caᵥα1), β (Caᵥβ) α2δ (Caᵥα2δ) and γ (Caᵥγ). The α1 subunits are the key porous forming units of the channel complex, being responsible for Ca²⁺ conduction and generation of Ca influx. The α2δ, β and γ subunits are auxiliary, although they are very important for the regulation of the channel since they increase the expression of α1 subunits in the plasma membrane as well as modulate their function resulting in functional diversity in different cell types. Based on their physiological and pharmacological properties, VGCC can be subdivided into low voltage-activated T-type (Caᵥ3.1, Caᵥ3.2, and Caᵥ3.3), and high voltage-activated L-(Caᵥ11.1 through Caᵥ1.4), N-(Caᵥ2.2), P/Q-(Caᵥ2.1), and R-(Caᵥ2.3) types, depending on the channel forming Caᵥα subunits. All of these five subclasses are found in the central and peripheral nervous systems. Regulation of intracellular calcium through activation of these VGCC plays obligatory roles in: 1) neurotransmitter release, 2) membrane depolarization and hyperpolarization, 3) enzyme activation and inactivation, and 4) gene regulation (Perret and Luo; Neurotherapeutics; 2009; 6; 679-692; Zamponl et al., 2015; Neumaler et al.; Prog. Neurobiol.; 2015; 129; 1-36). A large body of data has clearly indicated that VGCC are implicated in mediating various disease states including pain processing. Drugs interacting with the different calcium channel subtypes and subunits have been developed. Current therapeutic agents include drugs targeting L-type Caᵥ1.2 calcium channels, particularly 1,4-dihydropyridines, which are widely used in the treatment of hypertension. T-type (Caᵥ3) channels are the target of ethosuximide, widely used in absence epilepsy. Ziconotide, a peptide blocker of N-type (Caᵥ2.2) calcium channels, has been approved as a treatment of intractable pain.

The Caᵥ1 and Caᵥ2 subfamilies contain an auxiliary α2δ subunit which is the therapeutic target of the gabapentinoid drugs of value in certain epilepsies and chronic neuropathic pain (Perret and Luo, 2009; Vink and Alewood; British J. Pharmacol.; 2012; 167; 970-989). To date, there are four known α2δ subunits, each encoded by a unique gene and all possessing splice variants. Each α2δ protein is encoded by a single messenger RNA and is post-translationally cleaved and then linked by disulfide bonds. Four genes encoding α2δ subunits have now been cloned. α2δ-1 was initially cloned from skeletal muscle and shows a fairly ubiquitous distribution. The α2δ-2 and α2δ-3 subunits were subsequently cloned from brain. The most recently identified subunit, α2δ-4, is largely non-neuronal. The human α2δ-4 protein sequence shares 30, 32 and 61% identity with the human α2δ-1, α2δ-2 and α2δ-3 subunits, respectively. The gene structure of all α2δ subunits is similar. All α2δ subunits show several splice variants (Davies et al.; Trends Pharmacol. Sci.; 2007; 28; 220-228; Dolphin, A. C.; Nat. Rev. Neurosci.; 2012; 13; 542-555; Dolphin, A. C.; Biochim. Blophys. Acta; 2013; 1828; 1541-1549).

The Caᵥα2δ-1 subunit may play an important role in neuropathic pain development (Perret and Luo, 2009; Vink and Alewood, 2012). Biochemical data have indicated a significant Caᵥα2δ-1, but not Caᵥα2δ-2, subunit upregulation in the spinal dorsal horn, and DRG (dorsal root ganglia) after nerve injury that correlates with neuropathic pain development. In addition, blocking axonal transport of injury-induced DRG Caᵥα2δ-1 subunit to the central presynaptic terminals diminishes tactile allodynia in nerve injured animals, suggesting that elevated DRG Caᵥα2δ-1 subunit contributes to neuropathic allodynia.

The Caᵥα2δ-1 subunit (and the Caᵥα2δ-2, but not Caᵥα2δ-3 and Caᵥα2δ-4, subunits) is the binding site for gabapentin which has anti-allodynic/hyperalgesic properties in patients and animal models. Because injury-induced Caᵥα2δ-1 expression correlates with neuropathic pain, development and maintenance, and various calcium channels are known to contribute to spinal synaptic neurotransmission and DRG neuron excitability, injury-induced Caᵥα2δ-1 subunit upregulation may contribute to the initiation and maintenance of neuropathic pain by altering the properties and/or distribution of VGCC in the subpopulation of DRG neurons and their central terminals, therefore modulating excitability and/or synaptic neuroplasticity in the dorsal horn. Intrathecal antisense oligonucleotides against the Caᵥα2δ-1 subunit can block nerve injury-induced Caᵥα2δ-1 upregulation and prevent the onset of allodynia and reserve established allodynia.

As above mentioned, the α2δ subunits of VGCC form the binding site for gabapentin and pregabalin which are structural derivatives of the inhibitory neurotransmitter GABA although they do not bind to GABAA, GABAB, or benzodiazepine receptors, or alter GABA regulation in animal brain preparations. The binding of gabapentin and pregabalin to the Caᵥα2δ-1 subunit results in a reduction in the calcium-dependent release of multiple neurotransmitters, leading to efficacy and tolerability for neuropathic pain management. Gabapentinoids may also reduce excitability by inhibiting synaptogenesis (Perret and Luo, 2009; Vink and Alewood, 2012, Zamponi et al., 2015).

It is also known that Noradrenaline (NA), also called norepinephrine, functions in the human brain and body as a hormone and neurotransmitter. Noradrenaline exerts many effects and mediates a number of functions in living organisms. The effects of noradrenaline are mediated by two distinct super-families of receptors, named alpha- and beta-adrenoceptors. They are further divided into subgroups exhibiting specific roles in modulating behavior and cognition of animals. The release of the neurotransmitter noradrenaline throughout the mammalian brain is important for modulating attention, arousal, and cognition during many behaviors (Mason, S. T.; Prog. Neurobiol.; 1981; 16; 263-303).

The noradrenaline transporter (NET, SLC6A2) is a monoamine transporter mostly expressed in the peripheral and central nervous systems. NET recycles primarily NA, but also serotonin and dopamine, from synaptic spaces into presynaptic neurons. NET is a target of drugs treating a variety of mood and behavioral disorders, such as depression, anxiety, and attention-deficit/hyperactivity disorder (ADHD). Many of these drugs inhibit the uptake of NA into the presynaptic cells through NET. These drugs therefore increase the availability of NA for binding to postsynaptic receptors that regulate adrenergic neurotransmission. NET inhibitors can be specific. For example, the ADHD drug atomoxetine is a NA reuptake inhibitor (NRI) that is highly selective for NET. Reboxetine was the first NRI of a new antidepressant class (Kasper et al.; Expert Opin. Pharmacother.; 2000; 1; 771-782). Some NET inhibitors also bind multiple targets, increasing their efficacy as well as their potential patient population.

Endogenous, descending noradrenergic fibers impose analgesic control over spinal afferent circuitry mediating the transmission of pain signals (Ossipov et al.; J. Clin. Invest.; 2010; 120; 3779-3787). Alterations in multiple aspects of noradrenergic pain processing have been reported, especially in neuropathic pain states (Ossipov et a., 2010; Wang et al.; J. Pain; 2013; 14; 845-853). Numerous studies have demonstrated that activation of spinal α2-adrenergic receptors exerts a strong antinociceptive effect. Spinal clonidine blocked thermal and capsaicin-induced pain in healthy human volunteers (Ossipov et a., 2010). Noradrenergic reuptake inhibitors have been used for the treatment of chronic pain for decades: most notably the tricyclic antidepressants, amitriptyline, and nortriptyline. Once released from the presynaptic neuron, NA typically has a short-lived effect, as much of it is rapidly transported back into the nerve terminal. In blocking the reuptake of NA back into the presynaptic neurons, more neurotransmitter remains for a longer period of time and is therefore available for interaction with pre- and postsynaptic α₂-adrenergic receptors (AR). Tricyclic antidepressants and other NA reuptake inhibitors enhance the antinociceptive effect of opioids by increasing the availability of spinal NA. The α₂A-AR subtype is necessary for spinal adrenergic analgesia and synergy with opioids for most agonist combinations in both animal and humans (Chabot-Doré et al.; Neuropharmacology; 2015; 99; 285-300). A selective upregulation of spinal NET in a rat model of neuropathic pain with concurrent downregulation of serotonin transporters has been shown (Fairbanks et al.; Pharmacol. Ther.; 2009; 123; 224-238). Inhibitors of NA reuptake such as nisoxetine, nortriptyline and maprotiline and dual inhibitors of the noradrenaline and serotonin reuptake such as imipramine and milnacipran produce potent anti-nociceptive effects in the formalin model of tonic pain. Neuropathic pain resulting from the chronic constriction injury of the sciatic nerve was prevented by the dual uptake inhibitor, venlafaxine. In the spinal nerve ligation model, amitriptyline, a non-selective serotonin and noradrenaline reuptake blocker, the preferential noradrenaline reuptake inhibitor, desipramine and the selective serotonin and noradrenaline reuptake inhibitors, milnacipran and duloxetine, produce a decrease in pain sensitivity whereas the selective serotonin reuptake inhibitor, fluoxetine, is ineffective (Mochizucki, D.; Psychopharmacol.; 2004; Supplm. 1; S15-S19; Hartrick, C. T.; Expert Opin. Investig. Drugs; 2012; 21; 1827-1834). A number of nonselective investigational agents focused on noradrenergic mechanisms with the potential for additive or even synergistic interaction between multiple mechanisms of action are being developed (Hartrick, 2012).

Polypharmacology is a phenomenon in which a drug binds multiple rather than a single target with significant affinity. The effect of polypharmacology on therapy can be positive (effective therapy) and/or negative (side effects). Positive and/or negative effects can be caused by binding to the same or different subsets of targets; binding to some targets may have no effect. Multi-component drugs or multi-targeting drugs can overcome toxicity and other side effects associated with high doses of single drugs by countering biological compensation, allowing reduced dosage of each compound or accessing context-specific multitarget mechanisms. Because multitarget mechanisms require their targets to be available for coordinated action, one would expect synergies to occur in a narrower range of cellular phenotypes given differential expression of the drug targets than would the activities of single agents. In fact, it has been experimentally demonstrated that synergistic drug combinations are generally more specific to particular cellular contexts than are single agent activities, such selectivity is achieved through differential expression of the drugs' targets in cell types associated with therapeutic, but not toxic, effects (Lehar et al.; Nat. Biotechnol.; 2009; 27; 659-666).

In the case of chronic pain, which is a multifactorial disease, multi-targeting drugs may produce concerted pharmacological intervention of multiple targets and signaling pathways that drive pain. Because they actually make use of biological complexity, multi-targeting (or multi-component drugs) approaches are among the most promising avenues toward treating multifactorial diseases such as pain (Gilron et al.; Lancet Neurol.; 2013; 12(11); 1084-1095). In fact, positive synergistic interaction for several compounds, including analgesics, has been described (Schröder et al; J. Pharmacol. Exp. Ther.; 2011; 337; 312-320; Zhang et al.; Cell Death Dis.; 2014; 5; e1138; Gilron et al., 2013).

Given the significant differences in pharmacokinetics, metabolisms and bioavailability, reformulation of drug combinations (multi-component drugs) is challenging. Further, two drugs that are generally safe when dosed individually cannot be assumed to be safe in combination. In addition to the possibility of adverse drug-drug interactions, if the theory of network pharmacology indicates that an effect on phenotype may derive from hitting multiple targets, then that combined phenotypic perturbation may be efficacious or deleterious. The major challenge to both drug combination strategies is the regulatory requirement for each individual drug to be shown to be safe as an individual agent and in combination (Hopkins, A. L.; Nat. Chem. Biol.; 2008; 4; 682-690).

An alternative strategy for multitarget therapy is to design a single compound with selective polypharmacology (multi-targeting drug). It has been shown that many approved drugs act on multiple targets. Dosing with a single compound may have advantages over a drug combination in terms of equitable pharmacokinetics and biodistribution. Indeed, troughs in drug exposure due to incompatible pharmacokinetics between components of a combination therapy may create a low-dose window of opportunity where a reduced selection pressure can lead to drug resistance. In terms of drug registration, approval of a single compound acting on multiple targets faces significantly lower regulatory barriers than approval of a combination of new drugs (Hopkins, 2008).

Thus, the present invention discloses novel compounds having affinity for the α2δ subunits of voltage-gated calcium channels, preferably towards the α2δ-1 subunit of voltage-gated calcium channels, as well as inhibitory effect towards noradrenaline transporter (NET) and are, thus, more effective to pain, especially chronic pain.

There are two potentially important interactions between NET and α2δ-1 inhibition: 1) synergism in analgesia, thus reducing the risk of specific side effects; and 2) inhibition of pain-related affective comorbidities such as anxiety and/or depressive like behaviors (Nicolson et al.; Harv. Rev. Psychiatry; 2009; 17; 407-420).
1) Preclinical research has demonstrated that gabapentinoids attenuated pain-related behaviors through supraspinal activation of the descending noradrenergic system (Tanabe et al.; J. Neuroosci. Res.; 2008; Hayashida, K.; Eur. J. Pharmacol.; 2008; 598; 21-26). In consequence, the α2δ-1-related analgesia mediated by NA-induced activation of spinal α₂-adrenergic receptors can be potentiated by the inhibition of the NET. Some evidence from combination studies in preclinical models of neuropathic pain exist. Oral duloxetine with gabapentin was additive to reduce hypersensitivity induced by nerve injury in rats (Hayashida; 2008). The combination of gabapentin and nortriptyline drugs was synergic in mice submitted to orofacial pain and to peripheral nerve injury model (Miranda, H. F. et al.; J. Orofac. Pain; 2013; 27; 361-366; Pharmacology; 2015; 95; 59-64).
2) Drug modulation of NET and α2δ-1 has been shown to produce antidepressant and anti-anxiety effects respectively (Frampton, J. E.; CNS Drugs; 2014; 28; 835-854; Hajós, M. et al.; CNS Drug Rev.; 2004; 10; 23-44). In consequence, a dual drug that inhibited the NET and α2δ-1 subunit of VGCC may also stabilize pain-related mood impairments by acting directly on both physical pain and the possible mood alterations.

### SUMMARY OF THE INVENTION

The present invention discloses novel compounds with great affinity to the α2δ subunit of voltage-gated calcium channels, more specifically to the oα2δ1, as well as inhibitory effect towards the noradrenaline transporter (NET), thus resulting in a dual activity for treating pain and pain related disorders.

The main aspect of the present invention is related to compounds of general formula (I):

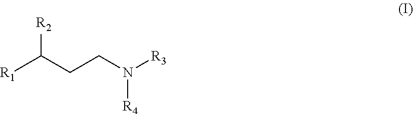

wherein:

R₁ is selected from an optionally substituted 5 or 6-membered aryl group or an optionally substituted 5 to 10-membered heteroaryl group having at least one heteroatom selected from the group of N, O or S;

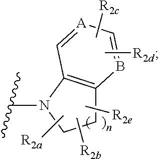

n is 1 or 2;

A and B independently represent a carbon atom leading to either —CH—, —CR_{2c}— or —CR_{2d}—; or a nitrogen atom with the proviso that if one is nitrogen the other is a carbon atom and with the proviso that when A and B are both carbon atoms, R₁ can not be phenyl;

R₂ₐ and R_{2b} are independently from one another a hydrogen atom or a branched or unbranched C₁₋₆ alkyl radical; or

R₂ₐ and R_{2b} being present at the same carbon atom can optionally form a spirocyclic structure;

R_{2c} and R_{2d} are independently from one another a hydrogen atom; a —(CH₂)ₘ—CN group m being 0 or 1; a halogen; a branched or unbranched C₁₋₆ alkyl radical; a C₁₋₆ alkylamino radical; an amino group; an hydroxyl group; a C₁₋₆ alkoxy radical; a C₁₋₆ haloalkoxy radical; an alkoxyalkyl C₁₋₆ radical; a C₃₋₆ cycloalkyl radical; a 5 or 6-membered heterocycloalkyl; an heterocydoalkylalkyl C₁₋₆; a C₁₋₆ haloalkyl radical; a —CF₃ group; an optionally substituted 5 or 6-membered aryl group; an arylalkyl radical C₁₋₆; an optionally substituted 5 to 10-membered heteroaryl group having at least one heteroatom selected from the group of N, O or S; or a heteroarylalkyl radical C₁₋₆;

R₂ₑ is a hydrogen atom; a═O group; or a branched or unbranched C₁₋₆ alkyl radical;

R₃ and R₄ are independently from one another a hydrogen atom or a branched or unbranched optionally substituted C₁₋₆ alkyl radical;

or a pharmaceutically acceptable salt, co-crystal, isomer, prodrug or solvate thereof.

It is also an aspect of the invention different processes for the preparation of compounds of formula (I).

Another aspect of the invention refers to the use of such compounds of general formula (I) for the treatment and/or prophylaxis of disorders mediated by the α2δ-1 subunit of voltage-gated calcium channels and/or noradrenaline transporter (NET). The compounds of the present invention are particularly suited for the treatment of pain, specially neuropathic pain, and pain related or pain derived conditions.

A further aspect of the invention is related to pharmaceutical compositions comprising one or more compounds of general formula (I) with at least one pharmaceutically acceptable excipient. The pharmaceutical compositions in accordance with the invention can be adapted in order to be administered by any route of administration, be it orally or parenterally, such as pulmonarily, nasally, rectally and/or intravenously. Therefore, the formulation in accordance with the invention may be adapted for topical or systemic application, particularly for dermal, subcutaneous, intramuscular, intra-articular, intraperitoneal, pulmonary, buccal, sublingual, nasal, percutaneous, vaginal, oral or parenteral application.

### DETAILED DESCRIPTION OF THE INVENTION

The invention first relates to compounds of general formula (I)

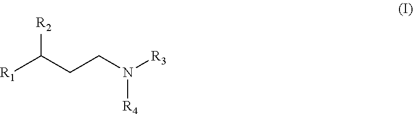

wherein:

R₁ is selected from an optionally substituted 5 or 6-membered aryl group or an optionally substituted 5 to 10-membered heteroaryl group having at least one heteroatom selected from the group of N, O or S;

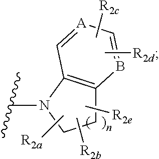

n is 1 or 2;

A and B independently represent a carbon atom leading to either —CH—, —CR_{2c}— or —CR_{2d}—; or a nitrogen atom the proviso that if one is nitrogen the other is a carbon atom and with the proviso that when A and B are both carbon atoms, R₁ can not be phenyl;

R₂ₐ and R_{2b} are independently from one another a hydrogen atom or a branched or unbranched C₁₋₆ alkyl radical; or

R₂ₐ and R_{2b} being present at the same carbon atom as substituents form a spirocyclic structure;

R_{2c} and R_{2d} are independently from one another a hydrogen atom; a —(CH₂)ₘ—CN group m being 0 or 1; a halogen; a branched or unbranched C₁₋₆ alkyl radical; a C₁₋₆ alkylamino radical; an amino group; an hydroxy group; a C₁₋₆ alkoxy radical; a C₁₋₆ haloalkoxy radical; an alkoxyalkyl C₁₋₆ radical; a C₃₋₆ cycloalkyl radical; a 5 or 6-membered heterocycloalkyl; an heterocycloalkylalkyl C₁₋₆; a C₁₋₆ haloalkyl radical; a —CF₃ group; an optionally substituted 5 or 6-membered aryl group; a arylalkyl radical C₁₋₆; an optionally substituted 5 to 10-membered heteroaryl group having at least one heteroatom selected from the group of N, O or S; or a heteroarylalkyl radical C₁₋₆;

R₂ₑ is a hydrogen atom; a ═O group; or a branched or unbranched C₁₋₆ alkyl radical;

R₃ and R₄ are independently from one another a hydrogen atom or a branched or unbranched optionally substituted C₁₋₆ alkyl radical;

or a pharmaceutically acceptable salt, co-crystal, isomer, prodrug or solvate thereof.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The compounds of general formula (I) or their salts, co-crystals or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, co-crystals, solvates or prodrugs.

“Halogen” or “halo” as referred in the present invention represent fluorine, chlorine, bromine or iodine. When the term “halo” is combined with other substituents, such as for instance “C₁₋₆ haloalkyl” or “C₁₋₆ haloalkoxy” it means that the alkyl or alkoxy radical can respectively contain at least one halogen atom.

A leaving group is a group that in a heterolytic bond cleavage keeps the electron pair of the bond. Suitable leaving groups are well known in the art and include Cl, Br, I and —O—SO₂R′, wherein R′ is F, C₁₋₄-alkyl, C₁₋₄-haloalkyl, or optionally substituted phenyl. The preferred leaving groups are Cl, Br, I, tosylate, mesylate, nosylate, triflate, nonaflate and fluorosulphonate.

“C₁₋₆ alkyl”, as referred to in the present invention, are saturated aliphatic radicals. They may be linear or branched and are optionally substituted. C₁₋₆-alkyl as expressed in the present invention means an alkyl radical of 1, 2, 3, 4, 5 or 6 carbon atoms. Preferred alkyl radicals according to the present invention include but are not restricted to methyl, ethyl, propyl, n-propyl, isopropyl, butyl, n-butyl, tert-butyl, isobutyl, sec-butyl, 1-methylpropyl, 2-methylpropyl, 1,1-dimethylethyl, pentyl, n-pentyl, 1,1-dimethylpropyl, 1,2-dimethylpropyl, 2,2-dimethylpropyl, hexyl or 1-methylpentyl. The most preferred alkyl radical are C₁₋₄ alkyl, such as methyl, ethyl, propyl, n-propyl, isopropyl, butyl, n-butyl, tert-butyl, isobutyl, sec-butyl, 1-methylpropyl, 2-methylpropyl or 1,1-dimethylethyl. Alkyl radicals, as defined in the present invention, are optionally mono- or polysubstituted by substitutents independently selected from a halogen, C₁₋₆-alkoxy, C₁₋₆-alkyl, C₁₋₆-haloalkoxy, C₁₋₆-haloalkyl, —CN, trihaloalkyl or a hydroxyl group.

“C₁₋₆ alkylamino” group or radical as referred to in the present invention, comprises a linear or branched, optionally at least mono-substituted alkyl chain of 1 to 6 carbon atoms which is bonded to an amino group. The alkylamino radical is bonded to the molecule through the alkyl chain.

“C₁₋₆ alkoxy” group or radical as referred in the present invention is an alkyl group as defined above attached via oxygen linkage to the rest of the molecule. Examples of alkoxy include, but are not limited to methoxy, ethoxy, propoxy, butoxy, tert-butoxy.

An alkoxyalkyl C₁₋₆ group/radical as defined in the present invention, comprises a linear or branched, optionally at least mono-substituted alkyl chain of 1 to 6 atoms which is bonded to an alkoxy group, as defined above. The alkoxyalkyl is bonded to the molecule through the alkyl chain. A preferred alkoxyalkyl group/radical is a methoxymethyl group.

“C₃₋₆ Cycloalkyl” as referred to in the present invention, is understood as meaning saturated and unsaturated (but not aromatic), cyclic hydrocarbons having from 3 to 6 carbon atoms which can optionally be unsubstituted, mono- or polysubstituted. Examples for cycloalkyl radical preferably include but are not restricted to cyclopropyl, cyclobutyl, cyclopentyl or cyclohexyl. Cycloalkyl radicals, as defined in the present invention, are optionally mono- or polysubstituted by substitutents independently selected from a halogen, C₁₋₆-alkyl, C₁₋₆-alkoxy, C₁₋₆-haloalkoxy, C₁₋₆-haloalkyl, trihaloalkyl or a hydroxyl group.

A cycloalkylalkyl group/radical C₁₋₆, as defined in the present invention, comprises a linear or branched, optionally at least mono-substituted alkyl chain of 1 to 6 atoms which is bonded to a cycloalkyl group, as defined above. The cycloalkylalkyl radical is bonded to the molecule through the alkyl chain. A preferred cycloalkylalkyl group/radical is a cyclopropylmethyl group or a cyclopentylpropyl group, wherein the alkyl chain is optionally branched or substituted. Preferred substituents for cycloalkylalkyl group/radical, according to the present invention, are independently selected from a halogen, C₁₋₆-alkyl, C₁₋₆-alkoxy, C₁₋₆-haloalkoxy, C₁₋₆-haloalkyl, trihaloalkyl or a hydroxyl group.

“Heterocycloalkyl” as referred to in the present invention, is understood as meaning saturated and unsaturated (but not aromatic), generally 5 or 6 membered cyclic hydrocarbons which can optionally be unsubstituted, mono- or polysubstituted and which have at least one heteroatom in their structure selected from N, O or S. Examples for heterocycloalkyl radical preferably include but are not restricted to pyrroline, pyrrolidine, pyrazoline, aziridine, azetidine, tetrahydropyrrole, oxirane, oxetane, dioxetane, tetrahydropyrane, tetrahydrofurane, dioxane, dioxolane, oxazolidine, piperidine, piperazine, morpholine, azepane or diazepane. Heterocycloalkyl radicals, as defined in the present invention, are optionally mono- or polysubstituted by substitutents independently selected from a halogen, C₁₋₆-alkyl, C₁₋₆-alkoxy, C₁₋₆-haloalkoxy, C₁₋₆-haloalkyl, trihaloalkyl or a hydroxyl group.

A heterocycloalkylalkyl group/radical C₁₋₆, as defined in the present invention, comprises a linear or branched, optionally at least mono-substituted alkyl chain of 1 to 6 atoms which is bonded to a heterocycloalkyl group, as defined above. The heterocycloalkylalkyl radical is bonded to the molecule through the alkyl chain. A preferred heterocycloalkylalkyl group/radical is a piperidinylmethyl, piperidinylethyl group or a piperazinylmethyl group, wherein the alkyl chain is optionally branched or substituted. Preferred substituents for heterocycloalkylalkyl group/radical, according to the present invention, are independently selected from a halogen, C₁₋₆-alkyl, C₁₋₆-alkoxy, C₁₋₆-haloalkoxy, C₁₋₆-haloalkyl, trihaloalky or a hydroxyl group.

“Aryl” as referred to in the present invention, is understood as meaning ring systems with at least one aromatic ring but without heteroatoms even in only one of the rings. These aryl radicals may optionally be mono- or polysubstituted by substitutents independently selected from a halogen, branched or unbranched C₁₋₆-alkyl, C₁₋₆-alkoxy, C₁₋₆-haloalkoxy, C₁₋₆-haloalkyl, CN or a hydroxyl group. Preferred examples of aryl radicals include but are not restricted to phenyl, naphthyl, fluoranthenyl, fluorenyl, tetralinyl, indanyl or anthracenyl radicals, which may optionally be mono- or polysubstituted, if not defined otherwise. More preferably aryl in the context of the present invention are 4 or 6-membered ring systems optionally at least monosubstituted.

An arylalkyl radical C₁₋₆, as defined in the present invention, comprises a linear or branched, optionally at least mono-substituted alkyl chain of 1 to 6 carbon atoms which is bonded to an aryl group, as defined above. The arylalkyl radical is bonded to the molecule through the alkyl chain. A preferred arylalkyl radical is a benzyl group or a phenethyl group, wherein the alkyl chain is optionally branched or substituted. Preferred substituents for arylalkyl radicals, according to the present invention, are independently selected from a halogen, branched or unbranched C₁₋₆-alkyl, C₁₋₆-alkoxy, C₁₋₆-haloalkoxy, C₁₋₆-haloalkyl, trihaloalkyl, —CN or a hydroxyl group.

“Heteroaryl” as referred to in the present invention, is understood as meaning heterocyclic ring systems which have at least one aromatic ring and may optionally contain one or more heteroatoms from the group consisting of N, O or S and may optionally be mono- or polysubstituted by substituents independently selected from a halogen, branched or unbranched C₁₋₆-alkyl, C₁₋₆-alkoxy, C₁₋₆-haloalkoxy, C₁₋₆-haloalkyl, trihaloalkyl, CN or a hydroxyl group. Preferred examples of heteroaryls include but are not restricted to furan, benzofuran, thiophene, thiazole, pyrrole, pyridine, pyrimidine, pyridazine, pyrazine, quinoline, isoquinoline, phthalazine, triazole, pyrazole, imidazole, oxazole, isoxazole, oxadiazole, indole, benzotriazole, benzodioxolane, benzodioxane, benzimidazole, carbazole, indazole and quinazoline. More preferably heteroaryl in the context of the present invention are 5 or 6-membered ring systems optionally at least monosubstituted.

Heteroarylalkyl group/radical C₁₋₆ as defined in the present invention, comprises a linear or branched, optionally at least mono-substituted alkyl chain of 1 to 6 carbon atoms which is bonded to an heteroaryl group, as defined above. The heteroarylalkyl radical is bonded to the molecule through the alkyl chain. Preferred substituents for heteroarylalkyl radicals, according to the present invention, are independently selected from a halogen, C₁₋₆-alkyl, C₁₋₆-alkoxy, C₁₋₆-haloalkoxy, C₁₋₆-haloalkyl, trihaloalkyl, CN or a hydroxyl group.

“Heterocyclic ring” or “heterocyclic system”, as defined in the present invention, comprise any saturated, unsaturated or aromatic carbocyclic ring systems which are optionally at least mono-substituted and which contain at least one heteroatom as ring member. Preferred heteroatoms for these heterocyclyl groups are N, S or O. Preferred substituents for heterocyclyl radicals, according to the present invention, a halogen, branched or unbranched C₁₋₆-alkyl, C₁₋₆-alkoxy, C₁₋₆-haloalkoxy, C₁₋₆-haloalkyl, trihaloalkyl, CN or a hydroxyl group.

The term “C₁₋₃ alkylene” is understood as meaning a divalent alkyl group like —CH₂— or —CH₂—CH₂— or —CH₂—CH₂—CH₂—. An “alkylene” may also be unsaturated.

The term “condensed” according to the present invention means that a ring or ring-system is attached to another ring or ring-system, whereby the terms “annulated” or “annelated” are also used by those skilled in the art to designate this kind of attachment.

The term “ring system” according to the present invention refers to ring systems comprising saturated, unsaturated or aromatic carbocyclic ring systems which contain optionally at least one heteroatom as ring member and which are optionally at least mono-substituted. Said ring systems may be condensed to other carbocyclic ring systems such as aryl groups, heteroaryl groups, cycloalkyl groups, etc.

“Spirocyclic structure” according to the present invention is a bicyclic ring system structure having one single carbon atom as the only common member of the two rings.

The term “salt” is to be understood as meaning any form of the active compound according to the invention in which it assumes an ionic form or is charged and is coupled with a counter-ion (a cation or anion) or is in solution. By this are also to be understood complexes of the active compound with other molecules and ions, in particular complexes which are complexed via ionic interactions. The definition particularly includes physiologically acceptable salts, this term must be understood as equivalent to “pharmacologically acceptable salts”.

The term “pharmaceutically acceptable salts” in the context of this invention means any salt that is tolerated physiologically (normally meaning that it is not toxic, particularly as a result of the counter-ion) when used in an appropriate manner for a treatment, particularly applied or used in humans and/or mammals. These physiologically acceptable salts may be formed with cations or bases and, in the context of this invention, are understood to be salts formed by at least one compound used in accordance with the invention—normally an acid (deprotonated)—such as an anion and at least one physiologically tolerated cation, preferably inorganic, particularly when used on humans and/or mammals. Salts with alkali and alkali earth metals are particularly preferred, as well as those formed with ammonium cations (NH₄⁺). Preferred salts are those formed with (mono) or (di)sodium, (mono) or (di)potassium, magnesium or calcium. These physiologically acceptable salts may also be formed with anions or acids and, in the context of this invention, are understood as being salts formed by at least one compound used in accordance with the invention—normally protonated, for example in nitrogen—such as a cation and at least one physiologically tolerated anion, particularly when used on humans and/or mammals. This definition specifically includes in the context of this invention a salt formed by a physiologically tolerated acid, i.e. salts of a specific active compound with physiologically tolerated organic or inorganic acids—particularly when used on humans and/or mammals. Examples of this type of salts are those formed with: hydrochloric acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid.

The term “co-crystal” is to be understood as a crystalline material comprising two or more compounds at ambient temperature (20 to 25° C., preferably 20° C.), of which at least two are held together by weak interaction, wherein at least one of the compounds is a co-crystal former. Weak interaction is being defined as an interaction which is neither ionic nor covalent and includes for example: hydrogen bonds, van der Waals forces, and π-π interactions.

The term “solvate” is to be understood as meaning any form of the active compound according to the invention in which this compound has attached to it via non-covalent binding another molecule (most likely a polar solvent) especially including hydrates and alcoholates, e.g. methanolate.

The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the compounds of the invention: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a prodrug of a compound of formula (I) is within the scope of the invention. Particularly favored prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

In a particular and preferred embodiment of the invention, R₁ represents a thiophene, a thiazole or a phenyl. These groups may be optionally substituted by at least one substituent selected from halogen, C₁₋₆ alkyl, C₁₋₆-alkoxy, C₁₋₆-haloalkoxy, C₁₋₆-haloalkyl, trihaloalkyl, CN or a hydroxyl group. The thiophene or thiazole group can be attached to the main structure through different points of attachment. For instance, when R₁ represents thiophene this might be a 2-thiophene or 3-thiophene or when it represents thiazole it may represent a 2-thiazole, a 4-thiazole or a 5-thiazole.

Thus, in a particularly preferred embodiment R₁ represents a group selected from:

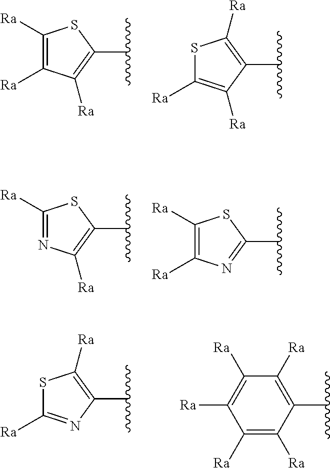

wherein each Rₐ independently represents a hydrogen atom, a halogen, C₁₋₆ alkyl, C₁₋₆-alkoxy, C₁₋₆-haloalkoxy, C₁₋₆-haloalkyl, trihaloalkyl, CN or a hydroxyl group. In another particular and preferred embodiment of the invention, R₂ is a group selected from:

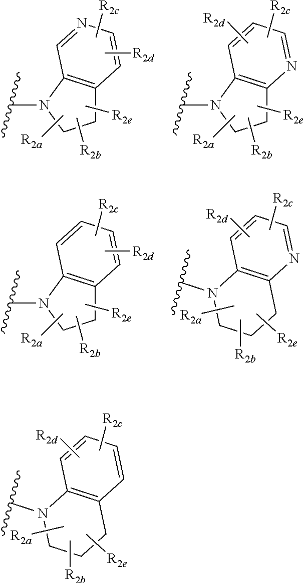

wherein R₂ₐ, R_{2b}, R_{2c}, R_{2d} and R₂ₑ are as defined above.

In another particular and preferred embodiment of the invention, R₂ₐ represents hydrogen, methyl or ethyl group.

In another particular and preferred embodiment of the invention, R_{2b} represents hydrogen, methyl or ethyl group.

In a particularly preferred embodiment of the invention, both R₂ₐ and R_{2b} independently represent hydrogen, methyl or ethyl.

In another particular and preferred embodiment of the invention, both R₂ₐ and R_{2b} represent a methyl group and are present in the same carbon atom as substituents.

In another particular and preferred embodiment of the invention, R₂ₐ and R_{2b}, are present in the same carbon atom as substituents and form a spirocyclopropyl.

In another particular embodiment R_{2c} and R_{2d} independently represent hydrogen, a —(CH₂)ₘ—CN group, m being 0 or 1; a halogen; a branched or unbranched C₁₋₆-alkyl radical; a C₁₋₆ alkylamino radical, a C₁₋₆ alkoxy radical; a C₁₋₆ haloalkoxy radical; an alkoxyalkyl C₁₋₆ radical; a C₃₋₆ cycloalkyl radical; a C₁₋₆ haloalkyl radical; —CF₃ group; an optionally substituted 5 or 6-membered aryl group; an arylalkyl radical C₁₋₆ or an optionally substituted 5 to 10-membered heteroaryl group having at least one heteroatom selected from the group of N, O or S.

In a still more particular and preferred embodiment R₂ and R_{2d} independently represent hydrogen, methyl, ethyl, isopropyl, halogen, methoxy, cyclopropyl, —CH₂—CN, —CN, —CH₂—N(CH₃)₂, methoxymethyl or a —CF₃ group.

In an even more particular and preferred embodiment R_{2c} and R_{2d} independently represent hydrogen, methyl, ethyl, isopropyl, halogen, methoxy, —CN, CF₃ or cyclopropyl.

In another particular and preferred embodiment of the invention, R_{2c} represents hydrogen, methyl, ethyl, isopropyl, fluoro, chloro, methoxy, —CN, CF₃ or cyclopropyl.

In another particular and preferred embodiment of the invention, R_{2d} represents hydrogen, methyl, ethyl, isopropyl, fluoro, chloro, methoxy, —CN, CF₃ or cyclopropyl.

In another particular and preferred embodiment of the invention, R₂ₑ represents a hydrogen atom; a methyl or an ethyl group.

In another particular and preferred embodiment of the invention, R₃ and R₄ independently represent hydrogen, methyl or ethyl.

In another particular and preferred embodiment of the invention, R₃ represents a hydrogen.

In another particular and preferred embodiment of the invention, R₄ represents a C₁₋₆ alkyl radical, more preferably methyl or ethyl.

In a particularly preferred embodiment of the invention, R₃ represents a hydrogen and R₄ represents a methyl.

A preferred embodiment of the invention is represented by a compound of formula (I):

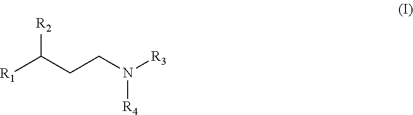

wherein R₁ represents a group selected from:

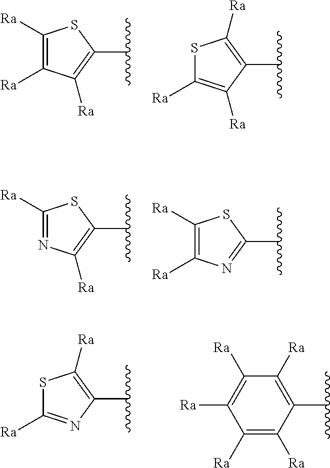

wherein each Rₐ independently represents a hydrogen atom, a halogen, C₁₋₆ alkyl, C₁₋₆-alkoxy, C₁₋₆-haloalkoxy, C₁₋₆-haloalkyl, trihaloalkyl, CN or a hydroxyl group;

R₂ is a group selected from:

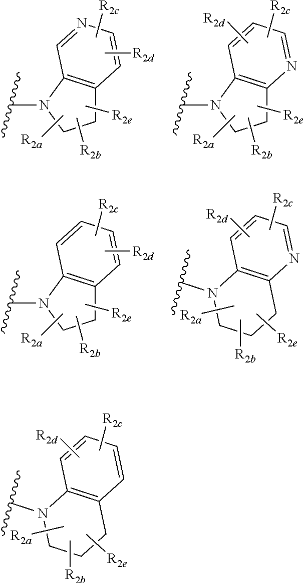

wherein

R₂ₐ represents hydrogen, methyl or ethyl group;

R_{2b} represents hydrogen, methyl or ethyl group;

R_{2c} and R_{2d} independently represent a independently represent a hydrogen, a —(CH)ₘ—CN group m being 0 or 1; a C₁₋₆ alkylamino radical, a halogen; a branched or unbranched C₁₋₆ alkyl radical; a C₁₋₆ alkoxy radical; an alkoxyalkyl C₁₋₆ radical; a C₃₋₆ cycloalkyl radical; a C₁₋₆ haloalkyl radical, a —CF₃ group; an optionally substituted 5 or 6-membered aryl group; an arylalkyl radical C₁₋₆ or an optionally substituted 5 to 10-membered heteroaryl group having at least one heteroatom selected from the group of N, O or S;

R₂ₑ is a hydrogen atom or a branched or unbranched C₁₋₆ alkyl radical;

R₃ and R₄ independently represent a hydrogen or a C₁₋₆ alkyl radical or a pharmaceutically acceptable salt, co-crystal, isomer, prodrug or solvate thereof.

A still more preferred embodiment of the invention is represented by a compound of formula (I):

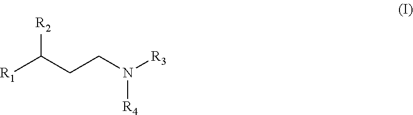

wherein R₁ represents a group selected from:

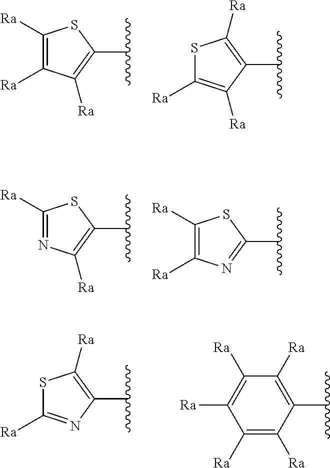

wherein each Rₐ independently represents a hydrogen atom, a halogen, C₁₋₆ alkyl, C₁₋₆-alkoxy, C₁₋₆-haloalkoxy, C₁₋₆-haloalkyl, trihaloalkyl, CN or a hydroxyl group;

R₂ is a group selected from:

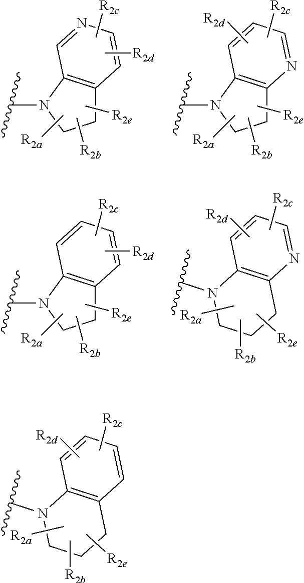

wherein:

R₂ₐ represents hydrogen, methyl or ethyl group;

R_{2b} represents hydrogen, methyl or ethyl group;

R_{2c} and R_{2d} independently represent hydrogen, methyl, ethyl, isopropyl, halogen, methoxy, cyclopropyl, —CH₂—CN, —CN, —CH₂—N(CH₃)₂, methoxymethyl or a —CF₃ group;

R₂ₑ is a hydrogen atom or a branched or unbranched C₁₋₆alkyl radical;

R₃ and R₄ independently represent a hydrogen, a methyl or ethyl;
or a pharmaceutically acceptable salt, co-crystal, isomer, prodrug or solvate thereof.

Another preferred embodiment of the invention is represented by a compound of formula (I):

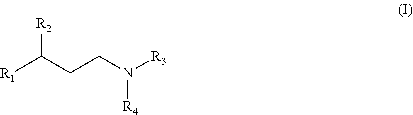

wherein R₁ represents a group selected from:

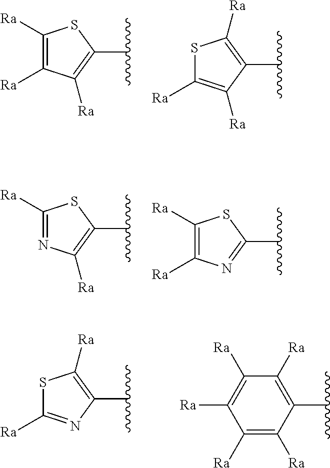

wherein each Rₐ independently represents a hydrogen atom, a halogen, C₁₋₆ alkyl, C₁₋₆-alkoxy, C₁₋₆-haloalkoxy, C₁₋₆-haloalkyl, trihaloalkyl, CN or a hydroxyl group;

R₂ is a group selected from:

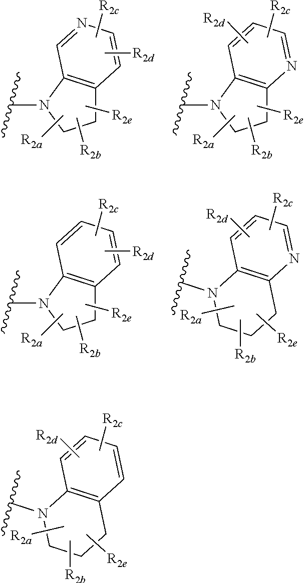

wherein:

R₂ₐ and R_{2b} represent hydrogen, methyl or ethyl;

R_{2c} and R_{2d} independently represent a hydrogen, a —(CH₂)ₘ—CN group m being 0 or 1; a C₁₋₆ alkylamino radical; a halogen; a branched or unbranched C₁₋₆ alkyl radical; a C₁₋₆ alkoxy radical; an alkoxyalkyl C₁₋₆ radical; a C₃₋₆ cycloalkyl radical; a C₁₋₆ haloalkyl radical; a CF₃; an optionally substituted 5 or 6-membered aryl group; an arylalkyl radical C₁₋₆ or an optionally substituted 5 to 10-membered heteroaryl group having at least one heteroatom selected from the group of N, O or S;

R₂ₑ is a hydrogen atom; or a branched or unbranched C₁₋₆ alkyl radical;

R₃ and R₄ independently represent a hydrogen, a methyl or ethyl;

or a pharmaceutically acceptable salt, co-crystal, isomer, prodrug or solvate thereof.

Another still more preferred embodiment of the invention is represented by a compound of formula (I):

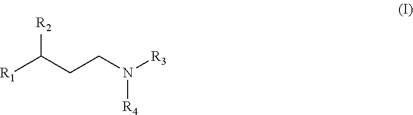

wherein R₁ represents a group selected from:

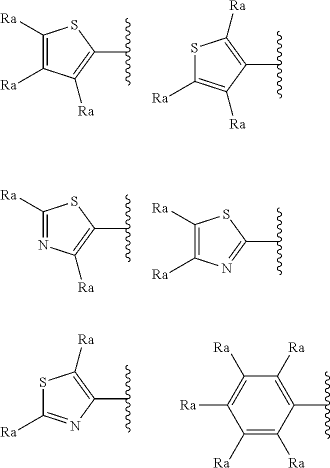

wherein each Rₐ independently represents a hydrogen atom, a halogen, C₁₋₆ alkyl, C₁₋₆ alkoxy, C₁₋₆-haloalkoxy, C₁₋₆haloalkyl, trihaloalkyl, —CN or a hydroxyl group;

R₂ is a group selected from:

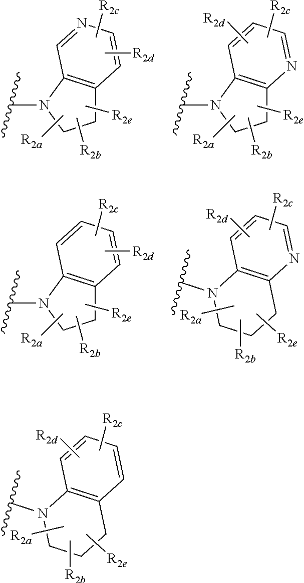

wherein:

R₂ₐ and R_{2b} represent hydrogen, methyl or ethyl;

R_{2c} and R_{2d} independently represent hydrogen, methyl, ethyl, isopropyl, halogen, methoxy, cyclopropyl, —CH₂—CN, —CN, —CH₂—N(CH₃)₂, methoxymethyl or a —CF₃ group;

R₂ₑ is a hydrogen atom; or a branched or unbranched C₁₋₆alkyl radical;

R₃ and R₄ independently represent a hydrogen, a methyl or ethyl;

or a pharmaceutically acceptable salt, co-crystal, isomer, prodrug or solvate thereof.

Another preferred embodiment of the invention is represented by a compound of formula (I):

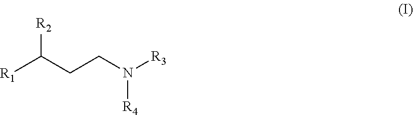

wherein R₁ represents a group selected from:

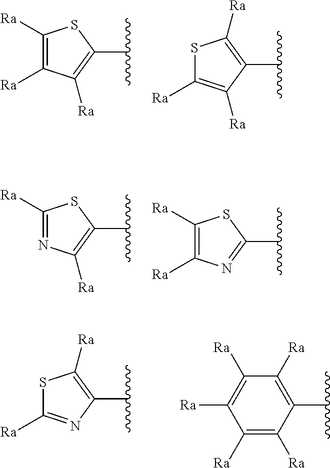

wherein each Rₐ independently represents a hydrogen atom, a halogen, C₁₋₆ alkyl, C₁₋₆-alkoxy, C₁₋₆-haloalkoxy, C₁₋₆-haloalkyl, trihaloalkyl, CN or a hydroxyl group;

R₂ is a group selected from:

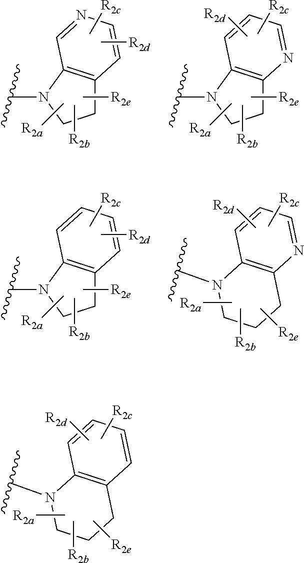

wherein:

R₂ₐ and R_{2b} are present in the same carbon atom as substituents and both represent a methyl group or form a spiro derivative, preferably a spirocyclopropyl;

R_{2c} and R_{2d} independently represent a hydrogen, a —(CH₂)ₘ—CN group m being 0 or 1; a C₁₋₆ alkylamino radical; a halogen; a branched or unbranched C₁₋₆ alkyl radical; a C₁₋₆ alkoxy radical; an alkoxyalkyl C₁₋₆ radical; a C₃₋₆ cycloalkyl radical; a C₁₋₆ haloalkyl radical; —CF₃ group; an optionally substituted 5 or 6-membered aryl group; an arylalkyl radical C₁₋₆ or an optionally substituted 5 to 10-membered heteroaryl group having at least one heteroatom selected from the group of N, O or S;

R₂ is a hydrogen atom; or a branched or unbranched C₁₋₆alkyl radical;

R₃ and R₄ independently represent a hydrogen, a methyl or ethyl;

or a pharmaceutically acceptable salt, co-crystal, isomer, prodrug or solvate thereof.

Another preferred embodiment of the invention is represented by a compound of formula (I):

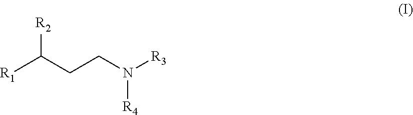

wherein R₁ represents a group selected from:

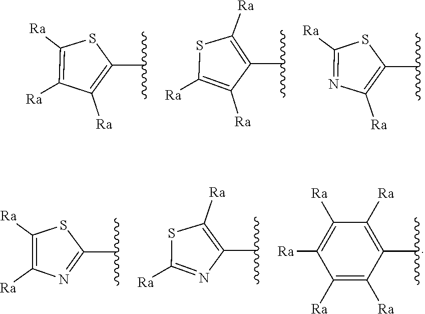

wherein each Rₐ independently represents a hydrogen atom, a halogen, C₁₋₆ alkyl, C₁₋₆-alkoxy, C₁₋₆-haloalkoxy, C₁₋₆-haloalkyl, trihaloalkyl, CN or a hydroxyl group;

R₂ is a group selected from:

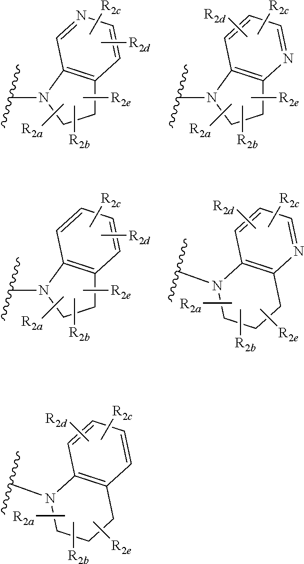

wherein:

R₂ₐ and R_{2b} represent a methyl group and are present in the same carbon atom as substituents;

R_{2c} and R_{2d} independently represent a hydrogen, a —(CH₂)ₘ—CN group m being 0 or 1; a C₁₋₆ alkylamino radical; a halogen; a branched or unbranched C₁₋₆ alkyl radical; a C₁₋₆ alkoxy radical; an alkoxyalkyl Ca₁₋₆ radical; a C₃₋₆ cycloalkyl radical; a C₁₋₆ haloalkyl radical; —CF₃; an optionally substituted 5 or 6-membered aryl group; an arylalkyl radical C₁₋₆; an optionally substituted 5 to 10-membered heteroaryl group having at least one heteroatom selected from the group of N, O or S;

R₂ₑ is a hydrogen atom; or a branched or unbranched C₁₋₆ alkyl radical;

R₃ and R₄ independently represent a hydrogen, a methyl or ethyl;

or a pharmaceutically acceptable salt, co-crystal, isomer, prodrug or solvate thereof.

Another still more preferred embodiment of the invention is represented by a compound of formula (I):

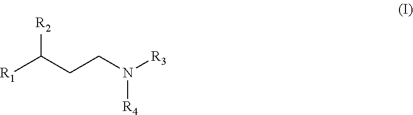

wherein R₁ represents a group selected from:

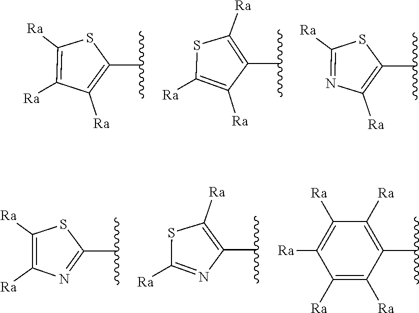

wherein each Rₐ independently represents a hydrogen atom, a halogen, C₁₋₆ alkyl, C₁₋₆-alkoxy, C₁₋₆-haloalkoxy, C₁₋₆-haloalkyl, trihaloalkyl, CN or a hydroxyl group;

R₂ is a group selected from:

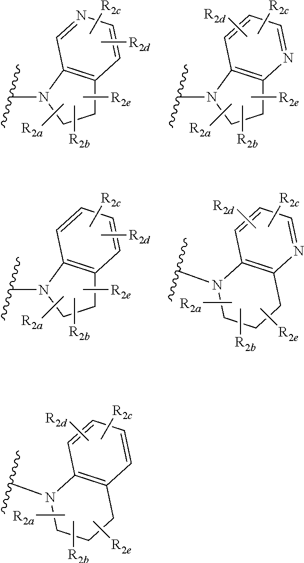

wherein:

R₂ₐ and R_{2b} are present in the same carbon atom as substituents and form a spiro structure, preferably a spirocyclopropyl;

R_{2c} and R_{2d} independently represent hydrogen, methyl, ethyl, isopropyl, halogen, methoxy, cyclopropyl, —CH₂—CN, —CN, —CH₂—N(CH₃)₂, methoxymethyl or a —CF₃ group

R₂ₑ is a hydrogen atom; or a branched or unbranched C₁₋₆ alkyl radical;

R₃ and R₄ independently represent a hydrogen, a methyl or ethyl;

or a pharmaceutically acceptable salt, co-crystal, isomer, prodrug or solvate thereof.

Another still more preferred embodiment of the invention is represented by a compound of formula (I):

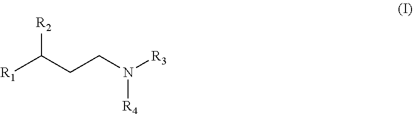

wherein R₁ represents a group selected from:

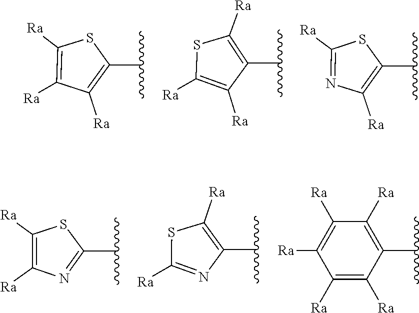

wherein each Rₐ independently represents a hydrogen atom, a halogen, C₁₋₆ alkyl, C₁₋₆-alkoxy, C₁₋₆-haloalkoxy, C₁₋₆-haloalkyl, trihaloalkyl, CN or a hydroxyl group;

R₂ is a group selected from:

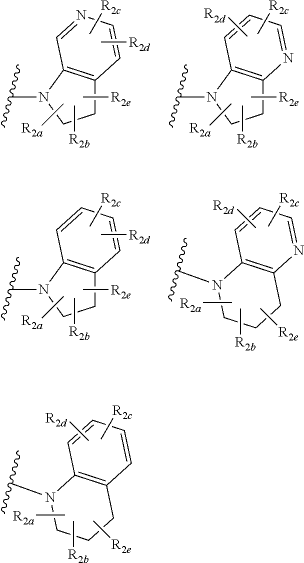

wherein:

R₂ₐ and R_{2b} represent a methyl group and are present in the same carbon atom as substituents;

R_{2c} and R_{2d} independently represent hydrogen, methyl, ethyl, isopropyl, halogen, methoxy, cyclopropyl, —CH₂—CN, —CN, —CH₂—N(CH₃)₂, methoxymethyl or a —CF₃ group;

R₂ is a hydrogen atom; or a branched or unbranched C₁₋₆ alkyl radical;

R₃ and R₄ independently represent a hydrogen, a methyl or ethyl;

or a pharmaceutically acceptable salt, co-crystal, isomer, prodrug or solvate thereof.

A further embodiment of the invention is related to compounds of formula (I) having the following subformula (Iaa), (Iab), (Iac) or (Iad):

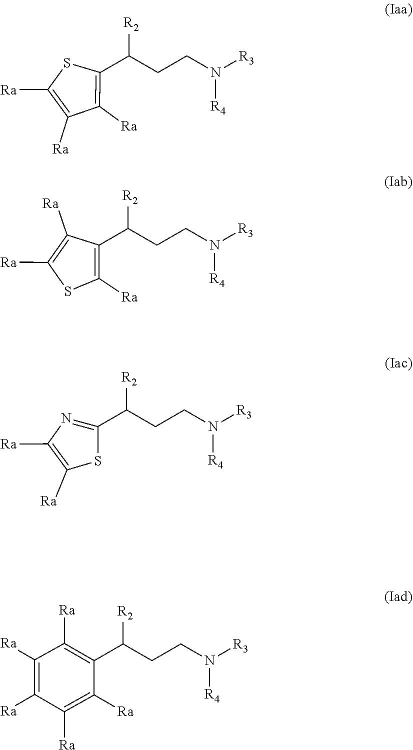

wherein R₂, R₃, R₄, and Rₐ are as defined above

Still another embodiment of the invention is related to compounds of formula (I) having the following subformula (Iba), (Ibb), (Ibc), (Ibd) or (Ibe):

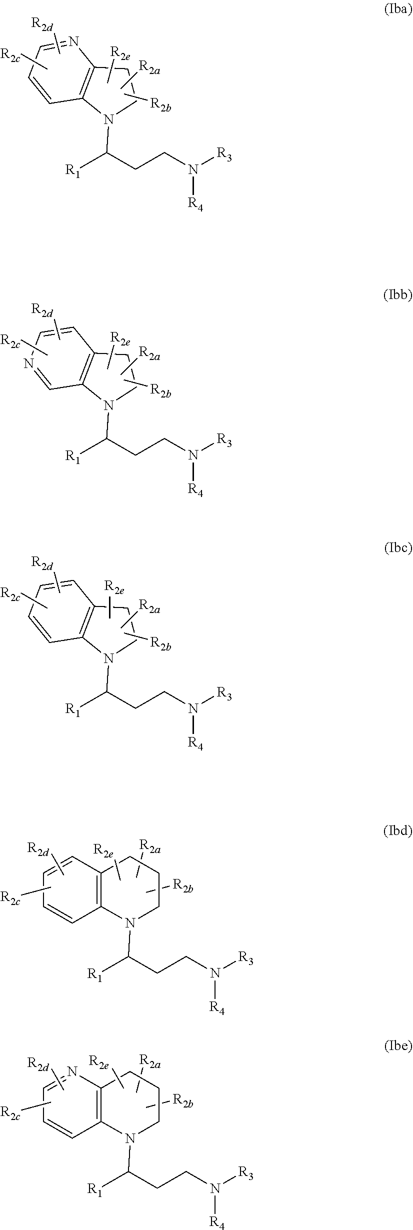

wherein R₁, R₂ₐ, R_{2b}, R_{2c}, R_{2d}, R₂ₑ, R₃ and R₄ are as defined above.

The compounds of the present invention represented by the above described formula (I) may include enantiomers depending on the presence of chiral centers or isomers depending on the presence of double bonds (e.g. Z, E). The single isomers, enantiomers or diastereoisomers and mixtures thereof fall within the scope of the present invention.

Among all the compounds described in the general formula (I), the following compounds are preferred for showing an inhibitory effect towards the α2δ-1 of voltage-gated calcium channels (VGCC) and noradrenaline transporter (NET):
[2] 3-(Indolin-1-yl)-N-methyl-3-(thiophen-2-yl)propan-1-amine;
[2] 3-(2,3-Dihydro-1H-pyrrolo[2,3-c]pyridin-1-yl)-N-methyl-3-(thiophen-2-yl)propan-1-amine;
[3] 3-(2,3-Dihydro-1H-pyrrolo[3,2-b]pyridin-1-yl)-N-methyl-3-(thiophen-2-yl)propan-1-amine;
[4] 3-(3,4-Dihydroquinolin-1 (2H)-yl)-N-methyl-3-(thiophen-2-yl)propan-1-amine;
[5] 3-(3,4-Dihydro-1,5-naphthyridin-1 (2H)-yl)-N-methyl-3-(thiophen-2-yl)propan-1-amine;
[6] 3-(2,3-Dihydro-1H-pyrrolo[3,2-b]pyridin-1-yl)-3-(5-fluorothiophen-2-yl)-N-methylpropan-1-amine;
[7] 3-(2,3-Dihydro-1H-pyrrolo[3,2-b]pyridin-1-yl)-N-methyl-3-phenylpropan-1-amine;
[8] 3-(2,3-Dihydro-1H-pyrrolo[3,2-b]pyridin-1-yl)-N-ethyl-3-(thiophen-2-yl)propan-1-amine;
[9] 3-(2,3-Dihydro-1H-pyrrolo[3,2-b]pyridin-1-yl)-3-(thiophen-2-yl)propan-1-amine;
[10] 3-(2,3-Dihydro-1H-pyrrolo[3,2-b]pyridin-1-yl)-N-methyl-3-(thiophen-3-yl)propan-1-amine;
[11] N-methyl-3-(5-methyl-2,3-dihydro-1H-pyrrolo[3,2-b]pyridin-1-yl)-3-(thiophen-2-yl)propan-1-amine;
[12] 3-(3,3-Dimethyl-2,3-dihydro-1H-pyrrolo[3,2-b]pyridin-1-yl)-N-methyl-3-(thiophen-2-yl)propan-1-amine;
[13] 3-(3,3-Dimethyl-2,3-dihydro-1H-pyrrolo[3,2-b]pyridin-1-yl)-N-methyl-3-(thiophen-3-yl)propan-1-amine;
[14] N-methyl-3-(6-methyl-2,3-dihydro-1H-pyrrolo[3,2-b]pyridin-1-yl)-3-(thiophen-3-yl)propan-1-amine;
[15] 3-(2,3-Dihydro-1H-pyrrolo[3,2-b]pyridin-1-yl)-N,N-dimethyl-3-(thiophen-2-yl)propan-1-amine;
[16] (S)-3-(2,3-Dihydro-1H-pyrrolo[3,2-b]pyridin-1-yl)-N-methyl-3-(thiophen-2-yl)propan-1-amine;
[17] (R)-3-(2,3-Dihydro-1H-pyrrolo[3,2-b]pyridin-1-yl)-N-methyl-3-(thiophen-2-yl)propan-1-amine;
[18] (R)-3-(2,3-Dihydro-1H-pyrrolo[3,2-b]pyridin-1-yl)-3-(5-fluorothiophen-2-yl)-N-methylpropan-1-amine;
[19] (S)-3-(2,3-Dihydro-1H-pyrrolo[3,2-b]pyridin-1-yl)-3-(5-fluorothiophen-2-yl)-N-methylpropan-1-amine;
[20] (R)-3-(3,3-Dimethyl-2,3-dihydro-1H-pyrrolo[3,2-b]pyridin-1-yl)-N-methyl-3-(thiophen-2-yl)propan-1-amine;
[21] (S)-3-(3,3-Dimethyl-2,3-dihydro-1H-pyrrolo[3,2-b]pyridin-1-yl)-N-methyl-3-(thiophen-2-yl)propan-1-amine;
[22] (R)-3-(2,3-Dihydro-1H-pyrrolo[3,2-b]pyridin-1-yl)-N-methyl-3-(thiophen-3-yl)propan-1-amine;
[23] (S)-3-(2,3-Dihydro-1H-pyrrolo[3,2-b]pyridin-1-yl)-N-methyl-3-(thiophen-3-yl)propan-1-amine;
[24] (R)-3-(3,3-Dimethyl-2,3-dihydro-1H-pyrrolo[3,2-b]pyrridin-1-yl)-N-methyl-3-(thiophen-3-yl)propan-1-amine;
[25] (S)-3-(3,3-Dimethyl-2,3-dihydro-1H-pyrrolo[3,2-b]pyrridin-1-yl)-N-methyl-3-(thiophen-3-yl)propan-1-amine;
[26] (R)-3-(6-Fluoro-2,3-dihydro-1H-pyrrolo[3,2-b]pyridin-1-yl)-N-methyl-3-(thiophen-2-yl)propan-1-amine;
[27] (S)-3-(6-Fluoro-2,3-dihydro-1H-pyrrolo[3,2-b]pyridin-1-yl)-N-methyl-3-(thiophen-2-yl)propan-1-amine;
[28] (R)-3-(6-Fluoro-2,3-dihydro-1H-pyrrolo[3,2-b]pyridin-1-yl)-N-methyl-3-(thiophen-3-yl)propan-1-amine;
[29] 25 [29] (S)-3-(6-Fluoro-2,3-dihydro-1H-pyrrolo[3,2-b]pyridin-1-yl)-N-methyl-3-(thiophen-3-yl)propan-1-amine;
[30] (R)-3-(6-Methoxy-2,3-dihydro-1H-pyrrolo[3,2-b]pyridin-1-yl)-N-methyl-3-(thiophen-2-yl)propan-1-amine;
[31] (S)-3-(6-Methoxy-2,3-dihydro-1H-pyrrolo[3,2-b]pyridin-1-yl)-N-methyl-3-(thiophen-2-yl)propan-1-amine;
[32] (R)-3-(6-Ethyl-2,3-dihydro-1H-pyrrolo[3,2-b]pyridin-1-yl)-N-methyl-3-(thiophen-2-yl)propan-1-amine;
[33] [33](S)-3-(6-Ethyl-2,3-dihydro-1H-pyrrolo[3,2-b]pyrridin-1-yl)-N-methyl-3-(thiophen-2-yl)propan-1-amine;
[34] 35 [34] (R)—N-methyl-3-(thiophen-2-yl)-3-(3,3,5-trimethyl-2,3-dihydro-1H-pyrrolo[3,2-b]pyridin-1-yl)propan-1-amine;
[35] (S)—N-methyl-3-(thiophen-2-yl)-3-(3,3,5-trimethyl-2,3-dihydro-1H-pyrrolo[3,2-b]pyridin-1-yl)propan-1-amine;
[36] (R)-3-(3-Chlorothiophen-2-yl)-3-(2,3-dihydro-1H-pyrrolo[3,2-b]pyridin-1-yl)-N-methylpropan-1-amine;
[37] (S)-3-(3-Chlorothiophen-2-yl)-3-(2,3-dihydro-1H-pyrrolo[3,2-b]pyridin-1-yl)-N-methylpropan-1-amine;
[38] (S)-3-(6-Isopropyl-2,3-dihydro-1H-pyrrolo[3,2-b]pyridin-1-yl)-N-methyl-3-(thiophen-2-yl)propan-1-amine:
[39] (R)-3-(6-Isopropyl-2,3-dihydro-1H-pyrrolo[3,2-b]pyridin-1-yl)-N-methyl-3-(thiophen-2-yl)propan-1-amine;
[40] (S)-3-(5-Chlorothiophen-2-yl)-3-(2,3-dihydro-1H-pyrrolo[3,2-b]pyridin-1-yl)-N-methylpropan-1-amine;
[41] (R)-3-(5-Chlorothiophen-2-yl)-3-(2,3-dihydro-1H-pyrrolo[3,2-b]pyridin-1-yl)-N-methylpropan-1-amine;
[42] (R)-3-(2,3-Dihydro-1H-pyrrolo[3,2-b]pyrridin-1-yl)-3-(2,5-dimethylthiophen-3-yl)-N-methylpropan-1-amine;
[43] (S)-3-(2,3-Dihydro-1H-pyrrolo[3,2-b]pyridin-1-yl)-3-(2,5-dimethylthiophen-3-yl)-N-methylpropan-1-amine;
[44] (R)-3-(2,3-Dihydro-1H-pyrrolo[3,2-b]pyridin-1-yl)-N-methyl-3-(5-methylthiophen-2-yl)propan-1-amine;
[45] (S)-3-(2,3-dihydro-1H-pyrrolo[3,2-b]pyridin-1-yl)-N-methyl-3-(5-methylthiophen-2-yl)propan-1-amine;
[46] (R)-3-(5-Isopropyl-2,3-dihydro-1H-pyrrolo[3,2-b]pyridin-1-yl)-N-methyl-3-(thiophen-3-yl)propan-1-amine;
[47] (S)-3-(5-Isopropyl-2,3-dihydro-1H-pyrrolo[3,2-b]pyridin-1-yl)-N-methyl-3-(thiophen-3-yl)propan-1-amine;
[48] (R)-3-(5-Isopropyl-2,3-dihydro-1H-pyrrolo[3,2-b]pyridin-1-yl)-N-methyl-3-(thiophen-2-yl)propan-1-amine;
[49] (S)-3-(5-Isopropyl-2,3-dihydro-1H-pyrrolo[3,2-b]pyridin-1-yl)-N-methyl-3-(thiophen-2-yl)propan-1-amine;
[50] (S)-3-(2,3-Dihydro-1H-pyrrolo[3,2-b]pyridin-1-yl)-N-methyl-3-(4-methylthiophen-3-yl)propan-1-amine;
[51] (R)-3-(2,3-Dihydro-1H-pyrrolo[3,2-b]pyridin-1-yl)-N-methyl-3-(4-methylthiophen-3-yl)propan-1-amine;
[52] (R)-3-(6-Cyclopropyl-2,3-dihydro-1H-pyrrolo[3,2-b]pyridin-1-yl)-N-methyl-3-(thiophen-3-yl)propan-1-amine;
[53] (S)-3-(6-Cyclopropyl-2,3-dihydro-1H-pyrrolo[3,2-b]pyridin-1-yl)-N-methyl-3-(thiophen-3-yl)propan-1-amine;
[54] (R)-3-(2,3-Dihydro-1H-pyrrolo[3,2-b]pyrridin-1-yl)-N-methyl-3-(thiazol-2-yl)propan-1-amine;
[55] 5 [55] (S)-3-(2,3-Dihydro-1H-pyrrolo[3,2-b]pyridin-1-yl)-N-methyl-3-(thiazol-2-yl)propan-1-amine;
[56] (R)-3-(2,3-Dihydro-1H-pyrrolo[3,2-b]pyridin-1-yl)-N-methyl-3-(4-methylthiophen-2-yl)propan-1-amine;
[57] (S)-3-(2,3-Dihydro-1H-pyrrolo[3,2-b]pyridin-1-yl)-N-methyl-3-(4-methylthiophen-2-yl)propan-1-amine;
[58] [58) (R)—N-methyl-3-(thiophen-3-yl)-3-(3,3,5-trimethyl-2,3-dihydro-1H-pyrrolo[3,2-b]pyridin-1-yl)propan-1-amine;
[59] (S)—N-methyl-3-(thiophen-3-yl)-3-(3,3,5-trimethyl-2,3-dihydro-1H-pyrrolo[3,2-b]pyridin-1-yl)propan-1-amine;
[60] (R)—N-methyl-3-(thiophen-3-yl)-3-(3,3,6-trimethyl-2,3-dihydro-1H-pyrrolo[3,2-b]pyridin-1-yl)propan-1-amine;
[61] (S)—N-methyl-3-(thiophen-3-yl)-3-(3,6-trimethyl-2,3-dihydro-1H-pyrrolo[3,2-b]pyridin-1-yl)propan-1-amine;
[62] (R)—N-methyl-3-(5-methyl-2,3-dihydro-1H-pyrrolo[3,2-b]pyridin-1-yl)-3-(thiophen-3-yl)propan-1-amine;
[63](S)—N-methyl-3-(5-methyl-2,3-dihydro-1H-pyrrolo[3,2-b]pyridin-1-yl)-3-(thiophen-3-yl)propan-1-amine;
[64] N-methyl-3-(thiophen-2-yl)-3-(3,3,5-trimethyl-2,3-dihydro-1H-pyrrolo[3,2-b]pyridin-1-yl)propan-1-amine;
[65] 3-(6-Fluoro-3,3-dimethyl-2,3-dihydro-1H-pyrrolo[3,2-b]pyridin-1-yl)-N-methyl-3-(thiophen-3-yl)propan-1-amine;
[66] 3-(6-Fluoro-3,3-dimethyl-2,3-dihydro-1H-pyrrolo[3,2-b]pyridin-1-yl)-N-methyl-3-(thiophen-2-yl)propan-1-amine;
[67] 3-(4-Fluoroindolin-1-yl)-N-methyl-3-(thiophen-2-yl)propan-1-amine;
[68] 3-(4,6-Difluoroindolin-1-yl)-N-methyl-3-(thiophen-2-yl)propan-1-amine;
[69] 3-(4-Methoxyindolin-1-yl)-N-methyl-3-(thiophen-2-yl)propan-1-amine;
[70] 3-(5-Chloro-3,3-dimethyl-2,3-dihydro-1H-pyrrolo[3,2-b]pyridin-1-yl)-N-methyl-3-(thiophen-3-yl)propan-1-amine;
[71] 3-(6-Fluoro-3,3,5-trimethyl-2,3-dihydro-1H-pyrrolo[3,2-b]pyridin-1-yl)-N-methyl-3-(thiophen-3-yl)propan-1-amine;
[72] 3-(5-Fluoroindolin-1-yl)-N-methyl-3-(thiophen-2-yl)propan-1-amine;
[73] 3-(6-Chloro-3,3-dimethyl-2,3-dihydro-1H-pyrrolo[3,2-b]pyridin-1-yl)-N-methyl-3-(thiophen-2-yl)propan-1-amine;
[74] 3-(2,3-Dihydro-1H-pyrrolo[3,2-c]pyridin-1-yl)-N-methyl-3-(thiophen-3-yl)propan-1-amine;
[75] 3-(3,3-Dimethyl-5-(trifluoromethyl)-2,3-dihydro-1H-pyrrolo[3,2-b]pyridin-1-yl)-N-methyl-3-(thiophen-2-yl)propan-1-amine;
[76] (R)-3-(6-Ethyl-2,3-dihydro-1H-pyrrolo[3,2-b]pyridin-1-yl)-N-methyl-3-(thiophen-3-yl)propan-1-amine;
[77] (S)-3-(6-Ethyl-2,3-dihydro-1H-pyrrolo[3,2-b]pyridin-1-yl)-N-methyl-3-(thiophen-3-yl)propan-1-amine;
[78] 3-(Indolin-1-yl)-N-methyl-3-(thiophen-3-yl)propan-1-amine;
[79] 3-(3,3-Dimethyl-5-(trifluoromethyl)-2,3-dihydro-1H-pyrrolo[3,2-b]pyridin-1-yl)-N-methyl-3-(thiophen-3-yl)propan-1-amine;
[80] 3-(6-Chloro-3,3-dimethyl-2,3-dihydro-1H-pyrrolo[3,2-b]pyridin-1-yl)-N-methyl-3-(thiophen-3-yl)propan-1-amine;
[81] N-methyl-3-(thiophen-2-yl)-3-(3,3,6-trimethyl-2,3-dihydro-1H-pyrrolo[3,2-b]pyridin-1-yl)propan-1-amine;
[82] (S)-3-(2,3-Dihydro-1H-pyrrolo[3,2-b]pyridin-1-yl)-3-(3-fluorophenyl)-N-methylpropan-1-amine;
[83] (R)-3-(2,3-Dihydro-1H-pyrrolo[3,2-b]pyridin-1-yl)-3-(3-fluorophenyl)-N-methylpropan-1-amine;
[84] (S)-3-(3,3-Dimethyl-2,3-dihydro-1H-pyrrolo[3,2-b]pyridin-1-yl)-3-(3-fluorophenyl)-N-methylpropan-1-amine;
[85] (R)-3-(3,3-Dimethyl-2,3-dihydro-1H-pyrrolo[3,2-b]pyridin-1-yl)-3-(3-fluorophenyl)-N-methylpropan-1-amine;
[86] 3-(3,3-Diethyl-2,3-dihydro-1H-pyrrolo[3,2-b]pyridin-1-yl)-N-methyl-3-(thiophen-3-yl)propan-1-amine;
[87] (R)-3-(6-Fluoro-3,3-dimethyl-2,3-dihydro-1H-pyrrolo[3,2-b]pyridin-1-yl)-N-methyl-3-(thiophen-3-yl)propan-1-amine;
[88] 30 [88] (S)-3-(6-Fluoro-3,3-dimethyl-2,3-dihydro-1H-pyrrolo[3,2-b]pyridin-1-yl)-N-methyl-3-(thiophen-3-yl)propan-1-amine;
[89] (S)-3-(6-Fluoro-3,3,5-trimethyl-2,3-dihydro-1H-pyrrolo[3,2-b]pyridin-1-yl)-N-methyl-3-(thiophen-3-yl)propan-1-amine;
[90] (R)-3-(6-Fluoro-3,3,5-trimethyl-2,3-dihydro-1H-pyrrolo[3,2-b]pyridin-1-yl)-N-methyl-3-(thiophen-3-yl)propan-1-amine;
[91] (R)—N-ethyl-3-(6-fluoro-2,3-dihydro-1H-pyrrolo[3,2-b]pyridin-1-yl)-3-(thiophen-3-yl)propan-1-amine;
[92] 3,3-Dimethyl-1-(3-(methylamino)-1-(thiophen-2-yl)propyl)indoline-6-carbonitrile;
[93] 3-(3,3-Dimethylindolin-1-yl)-N-methyl-3-(thiophen-2-yl)propan-1-amine;
[94] 1-(3-(Methylamino)-1-(thiophen-2-yl)propyl)indoline-6-carbonitrile;
[95] 1-(3-(Methylamino)-1-(thiophen-2-yl)propyl)indoline-4-carbonitrile;
[96] 1-(3-(Methylamino)-1-(thiophen-2-yl)propyl)indoline-5-carbonitrile;
[97] 3,3-Dimethyl-1-(3-(methylamino)-1-(thiophen-2-yl)propy)-2,3-dihydro-1-pyrrolo[3,2-b]pyridine-6-carbonitrile;
[98] N-methyl-3-(2-methylindolin-1-yl)-3-(thiophen-2-yl)propan-1-amine;
[99] 3-(5-Methoxy-3,3-dimethyl-2,3-dihydro-1H-pyrrolo[3,2-b]pyridin-1-yl)-N-methyl-3-(thiophen-2-yl)propan-1-amine;
[100] 3,3-Dimethyl-1-(3-(methylamino)-1-(thiophen-2-yl)propyl)indoline-4-carbonitrile;
[101] 1-(3-(Ethylamino)-1-(thiophen-2-yl)propyl)-3,3-dimethyl-2,3-dihydro-1H-pyrrolo[3,2-b]pyridine-6-carbonitrile;
[102] (S)—N-methyl-3-(6-methyl-2,3-dihydro-1H-pyrrolo[3,2-b]pyridin-1-yl)-3-(thiophen-3-yl)propan-1-amine;
[103] (R)—N-methyl-3-(6-methyl-2,3-dihydro-1H-pyrrolo[3,2-b]pyridin-1-yl)-3-(thiophen-3-yl)propan-1-amine;
[104] 3,3-Dimethyl-1-(3-(methylami no)-1-(thiophen-3-yl)propyl)-2,3-dihydro-1H-pyrrolo[3,2-b]pyridine-5-carbonitrile hydrochloride;
[105] (S)-3-(6-Fluoro-3,3-dimethyl-2,3-dihydro-1H-pyrrolo[3,2-b]pyridin-1-yl)-N-methyl-3-(thiophen-2-yl)propan-1-amine;
[106] (R)-3-(6-fluoro-3,3-dimethyl-2,3-dihydro-1H-pyrrolo[3,2-b]pyridin-1-yl)-N-methyl-3-(thiophen-2-yl)propan-1-amine;
[107] (S)-3-(3,3-Dimethyl-5-(trifluoromethyl)-2,3-dihydro-1H-pyrrolo[3,2-b]pyridin-1-yl)-N-methyl-3-(thiophen-3-yl)propan-1-amine;
[108] (R)-3-(3,3-dimethyl-5-(trifluoromethyl)-2,3-dihydro-1H-pyrrolo[3,2-b]pyridin-1-yl)-N-methyl-3-(thiophen-3-yl)propan-1-amine;
[109] (S)-3-(6-Chloro-3,3-dimethyl-2,3-dihydro-1H-pyrrolo[3,2-b]pyridin-1-yl)-N-methyl-3-(thiophen-2-yl)propan-1-amine;
[110] (R)-3-(6-chloro-3,3-dimethyl-2,3-dihydro-1H-pyrrolo[3,2-b]pyridin-1-yl)-N-methyl-3-(thiophen-2-yl)propan-1-amine;
[111] (S)-1-(3-(Methylamino)-1-(thiophen-2-yl)propyl)indoline-4-Carbonitrile;
[112] (S)-1-(3-(methylamino)-1-(thiophen-2-yl)propyl)indoline-4-carbonitrile;
[113] (S)-3-(5-Methoxy-3,3-dimethyl-2,3-dihydro-1H-pyrrolo[3,2-b]pyridin-1-yl)-N-methyl-3-(thiophen-2-yl)propan-1-amine;
[114] (R)-3-(5-methoxy-3,3-dimethyl-2,3-dihydro-1H-pyrrolo[3,2-b]pyridin-1-yl)-N-methyl-3-(thiophen-2-yl)propan-1-amine;
[115] (S)-3,3-Dimethyl-1-(3-(methylamino)-1-(thiophen-2-yl)propyl)-2,3-dihydro-1H-pyrrolo[3,2-b]pyridine-6-carbonitrile;
[116] (R)-3,3-dimethyl-1-(3-(methylamino)-1-(thiophen-2-yl)propyl)-2,3-dihydro-1H-pyrrolo[3,2-b]pyridine-6-carbonitrile;
[117] (S)—N-methyl-3-((R)-2-methylindolin-1-yl)-3-(thiophen-2-yl)propan-1-amine;
[118] (R)—N-methyl-3-((S)-2-methylindolin-1-yl)-3-(thiophen-2-yl)propan-1-amine;
[119] (S/R)—N-methyl-3-((S/R)-2-methylindolin-1-yl)-3-(thiophen-2-yl)propan-1-amine;
[120] (S)-1-(3-(Ethylamino)-1-(thiophen-2-yl)propyl)-3,3-dimethyl-2,3-dihydro-1H-pyrrolo[3,2-b]pyridine-6-carbonitrile and
[121] (R)-1-(3-(ethylamino)-1-(thiophen-2-yl)propyl)-3,3-dimethyl-2,3-dihydro-1H-pyrrolo[3,2-b]pyridine-6-carbonitrile.

In another aspect, the invention refers to the processes for the preparation of the compounds of general formula (I):

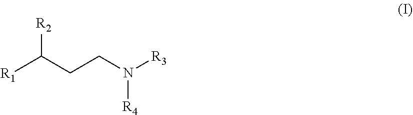

At least three different methods (A, B and C, described below) have been developed for obtaining the compounds of the invention.

Method A

First, a process is provided for obtaining a compound of formula (I):

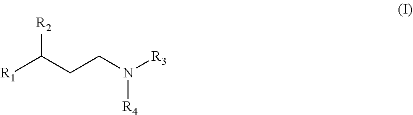

comprising the reduction of a carboxamido compound of formula (IV):

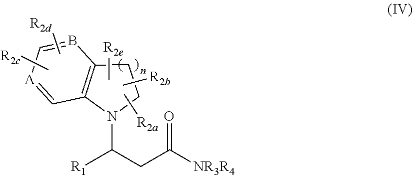

wherein R₁, R₂ₐ, R_{2b}, R_{2c}, R_{2d}, R₂ₑ, R₃, R₄, A, B and n are as defined in claim 1.

The reduction of a carboxamido compound of formula (IV) to obtain an amino compound of general formula (I) is carried out following conventional procedures described in the literature. As a way of example, the reduction can be performed using a hydride source such as borane-dimethyl sulfide complex, borane-tetrahydrofuran complex or lithium aluminium hydride in a suitable solvent such as tetrahydrofuran or diethyl ether, at a suitable temperature, preferably comprised between 0° C. and the reflux temperature.

In turn, a compound of formula (IV) can be prepared in two ways starting from a compound of formula (II):

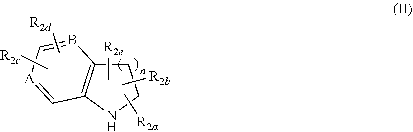

by reaction with either a compound of formula (IIIa) or (IIIb):

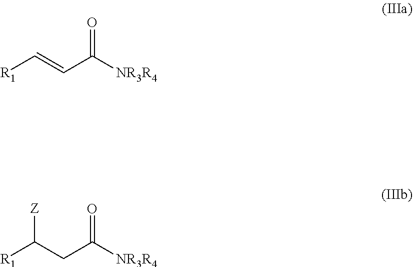

When an acrylamide of formula (IIIa) is used, the reaction is carried out by treating a compound of formula (II) with a compound of formula (IIIa) preferably in the presence of a strong base such as lithium diisopropylamide, lithium (or sodium or potassium) bis(trimethylsilyl)amide, n-butyllithium or sodium hydride. The Aza-Michael reaction is carried out preferably in a suitable aprotic solvent, such as tetrahydrofuran; at a suitable temperature comprised between −78° C. and room temperature, preferably cooling.

When a compound of formula IIIb is used, depending on the meaning of Z, the reaction is carried out differently:
When Z represents a leaving group (such as chloro, bromo, iodo, mesylate, tosylate, nosylate or triflate), the reaction is carried out under conventional alkylation conditions by treating a compound of formula (II) with an alkylating agent of formula (IIIb) preferably in the presence of a suitable base such as sodium hydride, potassium tert-butoxide, K₂CO₃ or Cs₂CO₃. The reaction is carried out in a suitable solvent, such as acetonitrile, tetrahydrofuran, dimethylformamide, dimethylacetamide, dimethylsulfoxide, dichloromethane or 1,4-dioxane; at a suitable temperature comprised between room temperature and the reflux temperature, preferably heating, or alternatively, the reactions can be carried out in a microwave reactor. Additionally, an activating agent such as sodium iodide can be used.
When Z represents OH, the reaction is carried out under conventional Mitsunobu conditions by treating a compound of formula (II) with an alcohol of formula (IIIb) in the presence of an azo compound such as 1,1′-(azodicarbonyl)dipiperidine (ADDP), diisopropylazodicarboxylate (DIAD) or diethyl azodicarboxylate (DEAD) and a phosphine such as tributylphosphine or triphenylphoshine. The reaction is carried out preferably in a suitable solvent, such as toluene or tetrahydrofuran; at a suitable temperature comprised between room temperature and the reflux temperature.

Alternatively, a compound of formula (IV) can be prepared from an ester precursor by treating a compound of formula (IV-Q)

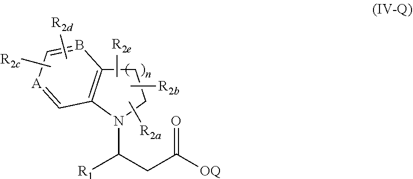

where Q represents an alkyl group or a 4-methoxyphenyl group, with an amine of formula (V),

HNR₃R₄ (V)

preferably using an excess of such amine, in a suitable solvent such as ethanol, methanol, isopropanol or mixtures with water, at a suitable temperature, preferably heating.

In addition, the conversion of a compound of formula (IV-Q) to a compound of formula (IV) can be conducted sequentially in 2 steps by hydrolyzing an ester of formula (IV-Q) to its corresponding acid of formula (IV-H)

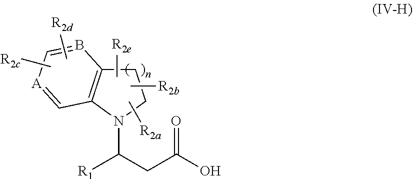

followed by reaction of the acid of formula (IV-H) with an amine of formula (V)

HNR₃R₄ (V)

to render a compound of formula (IV).

The hydrolysis of a compound of formula (IV-Q) to obtain a compound of formula (IV-H) is carried out under conventional reaction conditions by treating an ester of formula (IV-Q) with a base such as NaOH, LiOH or KOH, in a suitable solvent such as ethanol, methanol, THF, water or mixtures thereof; at a suitable temperature comprised between room temperature and the reflux temperature.

The amidation reaction between a compound of formula (IV-H) and an amine of formula (V) is carried out using a suitable coupling reagent such as N-(3-dimethylaminopropyl)-N′-ethylcarbodiimide (EDC), dicyclohexylcarbodiimide (DCC), N-[(dimethylamino)-1H-1,2,3-triazolo-[4,5-b]pyridin-1-ylmethylene]-N-methylmethanaminium hexafluorophosphate N-oxide (HATU) or N,N,N′,N′-tetramethyl-O-(1H-benzotriazol-1-yl)uronium hexafluorophosphate (HBTU), optionally in the presence of 1-hydroxybenzotriazole, optionally in the presence of an organic base such as N-methylmorpholine or N,N-diisopropylethylamine, in a suitable solvent such as dichloromethane or dimethylformamide, and at a suitable temperature, preferably at room temperature.

The ester compounds of formula (IV-Q) can in turn be synthesized by reacting a compound of formula (II) with a compound of formula (IIIa-Q) or (IIIb-Q)

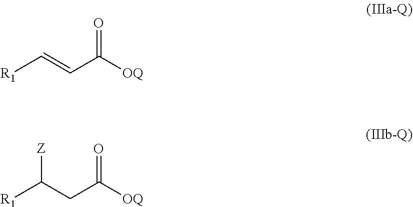

following the conditions described above for the preparation of a compound of formula (IV) from a compound of formula (II) and a compound of formula (IIIa) or (IIIb).

Following an analogous synthetic sequence, the compounds of formula (I) can be obtained in enantiopure form by reacting a compound of formula (II) with an homochiral compound of formula (IIIa-E) or (IIIb-E) (wherein E* represents a chiral auxiliary such as for example a chiral alcohol or a chiral 2-oxazolidinone)

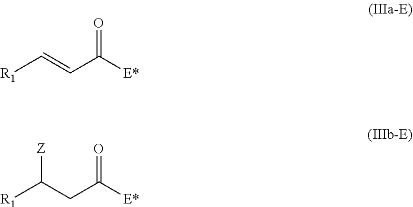

to render an homochiral compound of formula (IV-E)

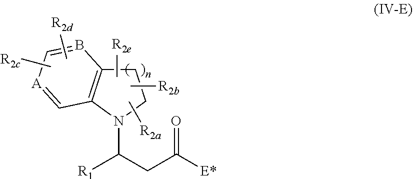

using the same reaction conditions described above, followed by reaction with an amine of formula (V) to render an homochiral compound of formula (IV). The reaction of a compound of formula (IV-E) with an amine of formula (V) is performed under the reaction conditions described above for the preparation of a compound of formula (IV) from a compound of formula (IV-Q). Finally, an enantiopure compound of formula (IV) is converted into an enantiopure compound of formula (I) by reduction following the conditions described above.

Alternatively an enantiopure compound of formula (IV-E) can be prepared from an acid of formula (IV-H) and the corresponding homochiral auxiliary using standard acylation conditions described in the literature, followed by separation of the diastereomeric mixture thus obtained by conventional methods, such as chromatography or crystallization.

The general synthetic route for preparing compounds of formula (I) according to method A as well as their intermediates, is represented in scheme 1:

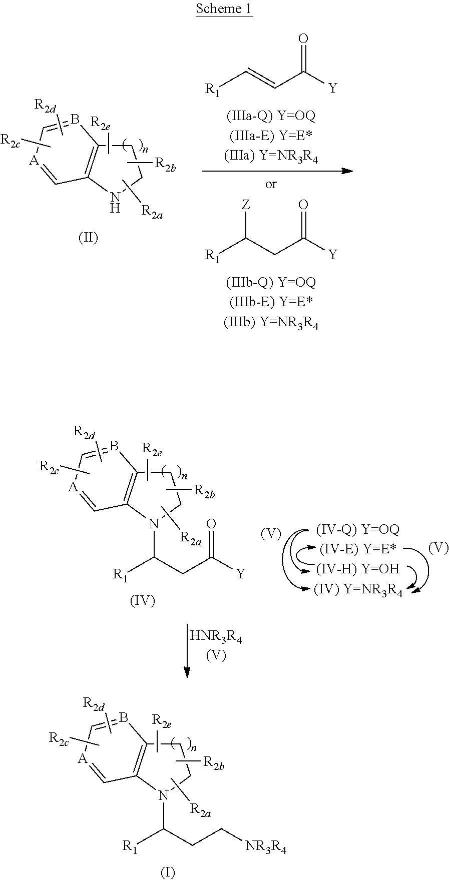

Method B

A second process for preparing a compound of formula (I) comprises the reaction of a compound of formula (II):

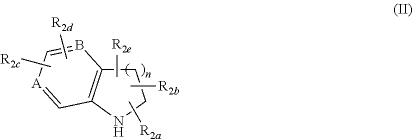

with a compound of formula (IIIc):

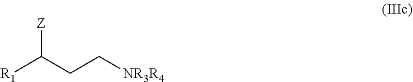

wherein R₁, R₂ₐ, R_{2b}, R_{2c}, R_{2d}, R₂ₑ, R₃, R₄, A, B and n are as defined in claim 1, and Z independently represents a leaving group or hydroxy group.

The reaction is preferably carried out under the same reaction conditions described above in method A for the synthesis of a compound of formula (IV) from a compound of formula II and a compound of formula (IIIb).

Method C

A third process for preparing a compound of formula (I) comprises the reaction of a compound of formula (VI-H) or (VI-G):

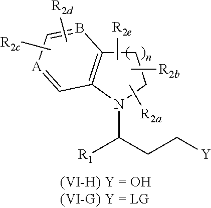

with a compound of formula (V):

HNR₃R₄ (V)

wherein R₁, R₂ₐ, R_{2b}, R_{2c}, R_{2d}, R₂ₑ, R₃, R₄, A, B and n are as defined in claim 1 and LG represents a suitable leaving group.

The alkylation reaction of a compound of formula (VI-G) wherein LG represents a leaving group (such as for instance chloro, bromo, iodo, mesylate, tosylate, nosylate or triflate) with an amine of formula (V) to render a compound of formula (I) is carried out in a suitable solvent, such as ethanol, dimethylformamide, dimethylsulfoxide or acetonitrile, preferably ethanol; preferably using an excess of amine (V) or optionally in the presence of a base such as K₂CO₃ or triethylamine; at a suitable temperature comprised between room temperature and the reflux temperature, preferably heating, or alternatively, the reactions can be carried out in a microwave reactor. Additionally, an activating agent such as sodium iodide or potassium iodide can be used.

The preparation of a compound of formula (I) from a compound of formula (VI-H) can be carried out following other conventional protocols described in the bibliography, such as:
a) oxidation of an alcohol of formula (VI-H) to the corresponding aldehyde followed by treatment with an amine of formula (V) under reductive amination conditions or
b) conversion of the hydroxy group into a phthalimido group by reacting a compound of formula (VI-H) with phthalimide under Mitsunobu conditions followed by hydrolysis and final derivatization (if required).

The alkylating agents of formula (VI-G) can be synthesized by converting an alcohol of formula (VI-H) to a leaving group following conventional procedures described in the literature. Alternatively, a compound of formula (VI-G) can be directly prepared in one step by reaction of a compound of formula (II) with a compound of formula (IIIc-G),

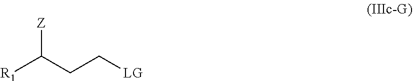

wherein R₁ is as defined in claim 1, and LG and Z independently represent a suitable leaving groups.

The reaction is carried out under the conditions described above for the preparation of a compound of formula (I) from a compound of formula (II) and a compound of formula (IIIc).

In turn, a compound of formula (VI-H) can be prepared by reduction of a compound of formula (IV-Q) or (IV-H). The reaction is carried out following conventional reduction procedures, using a hydride source such as sodium or lithium borohydride, borane-dimethyl sulfide complex, borane-tetrahydrofuran complex or lithium aluminium hydride. Equally, a compound of formula VI-H can be directly prepared in one step by reaction of a compound of formula II with a compound of formula IIIc-H,

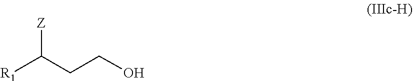

wherein R₁ is as defined in claim 1 and Z represents a suitable leaving group.

The synthetic routes described in methods B and C are summarized in scheme 2 below:

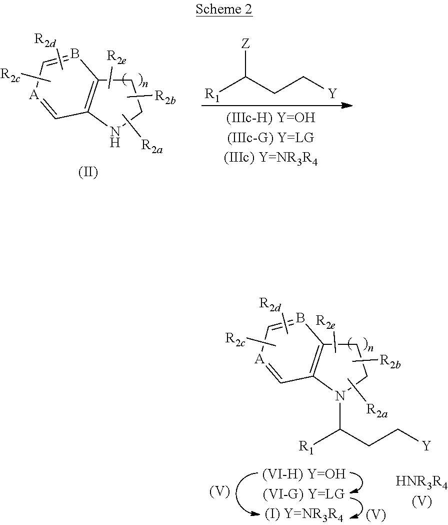

It is noted that compounds of formula (II), (IIIa-Q), (IIIa-E), (IIIa), (IIIb-Q), (IIIb-E), (IIIb), (IIIc-H), (IIIc-G), (IIIc) and (V) used in all three methods disclosed above are commercially available or can be synthesized following common procedures described in the literature.

Moreover, certain compounds of the present invention can also be obtained starting from other compounds of formula (I) by appropriate conversion reactions of functional groups, in one or several steps, using well-known reactions in organic chemistry under standard experimental conditions.

In some of the processes described above it may be necessary to protect the reactive or labile groups present with suitable protecting groups, such as for example Boc (tert-butoxycarbonyl), Teoc (2-(trimethylsilyl)ethoxycarbonyl) or benzyl for the protection of amino groups, and common silyl protecting groups for the protection of the hydroxyl group. The procedures for the introduction and removal of these protecting groups are well known in the art and can be found thoroughly described in the literature.

In addition, a compound of formula (I) can be obtained in enantiopure form by resolution of a racemic compound of formula (I) either by chiral preparative HPLC or by crystallization of a diastereomeric salt or co-crystal. Alternatively, the resolution step can be carried out at a previous stage, using any suitable intermediate.

The obtained reaction products may, if desired, be purified by conventional methods, such as crystallization and chromatography. Where the processes described below for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

Another particular aspect is represented by the intermediate compounds used for preparation of compounds of general formula (I).

In a particular embodiment, these intermediate compounds of general formula (I) are selected from:
6-Methoxy-2,3-dihydro-1H-pyrrolo[3,2-b]pyridine;
6-Fluoro-2,3-dihydro-1H-pyrrolo[3,2-b]pyridine;
5-Methyl-2,3-dihydro-1H-pyrrolo[3,2-b]pyridine;
5-Isopropyl-2,3-dihydro-1H-pyrrolo[3,2-b]pyridine;
6-Ethyl-2,3-dihydro-1H-pyrrolo[3,2-b]pyridine;
6-Isopropyl-2,3-dihydro-1H-pyrrolo[3,2-b]pyridine;
6-Cyclopropyl-2,3-dihydro-1H-pyrrolo[3,2-b]pyridine;
3,3,5-Trimethyl-2,3-dihydro-1H-pyrrolo[3,2-b]pyridine;
3,3,6-Trimethyl-2,3-dihydro-1H-pyrrolo[3,2-b]pyridine;
3,3-Dimethyl-5-(trifluoromethyl)-2,3-dihydro-1H-pyrrolo[3,2-b]pyridine;
(E)-Ethyl 3-(5-fluorothiophen-2-yl)acrylate;
(E)-3-(4-Methylthiophen-3-yl)acrylic acid;
2-(1,3-Dichloropropyl)thiophene;
6-Fluoro-3,3-dimethyl-2,3-dihydro-1H-pyrrolo[3,2-b]pyridine;
6-Fluoro-3,3,5-trimethyl-2,3-dihydro-1H-pyrrolo[3,2-b]pyridine;
3,3-Dimethyl-2,3-dihydro-1H-pyrrolo[3,2-b]pyridine-6-carbonitrile;
3,3-Diethyl-2,3-dihydro-1H-pyrrolo[3,2-b]pyridine;
5-Methoxy-3,3-dimethyl-2,3-dihydro-1H-pyrrolo[3,2-b]pyridine;
3,3-Dimethylindoline-4-carbonitrile;
(E)-4-Methoxyphenyl 3-(thiophen-2-yl)acrylate and
(E)-4-Methoxyphenyl 3-(thiophen-3-yl)acrylate.

Turning to another aspect, the invention also relates to the therapeutic use of the compounds of general formula (I). As mentioned above, compounds of general formula (I) show a strong affinity to subunit α2δ, especially to α2δ-1 subunit of voltage-gated calcium channels as well as to noradrenaline transporter (NET) and can behave as agonists, antagonists, inverse agonists, partial antagonists or partial agonists thereof. Therefore, compounds of general formula (I) are useful as medicaments.

They are suitable for the treatment and/or prophylaxis of diseases and/or disorders mediated by the α2δ especially the α2δ-1 subunit of voltage-gated calcium channels and/or noradrenaline transporter (NET). In this sense, compounds of formula (I) are suitable for the treatment and/or prophylaxis of pain, especially neuropathic pain, inflammatory pain, and chronic pain or other pain conditions involving allodynia and/or hyperalgesia, depression anxiety and attention-deficit-/hyperactivity disorder (ADHD).

The compounds of formula (I) are especially suited for the treatment of pain, especially neuropathic pain, inflammatory pain or other pain conditions involving allodynia and/or hyperalgesia. PAIN is defined by the International Association for the Study of Pain (IASP) as “an unpleasant sensory and emotional experience associated with actual or potential tissue damage, or described in terms of such damage (IASP, Classification of chronic pain, 2nd Edition, IASP Press (2002), 210). Even though pain is always subjective its causes or syndromes can be classified.

In a preferred embodiment compounds of the invention are used for the treatment and/or prophylaxis of allodynia and more specifically mechanical or thermal allodynia.

In another preferred embodiment compounds of the invention are used for the treatment and/or prophylaxis of hyperalgesia.

In yet another preferred embodiment compounds of the invention are used for the treatment and/or prophylaxis of neuropathic pain and more specifically for the treatment and/or prophylaxis of hyperpathia.

A related aspect of the invention refers to the use of compounds of formula (I) for the manufacture of a medicament for the treatment and/or prophylaxis of disorders and diseases mediated by the subunit α2δ, especially α2δ-1 subunit of voltage-gated calcium channels and/or noradrenaline transporter (NET), as explained before.

Another related aspect of the invention refers to a method for the treatment and/or prophylaxis of disorders and diseases mediated by the subunit α2δ, especially α2δ-1 subunit of voltage-gated calcium channels and/or noradrenaline transporter (NET), as explained before comprising the administration of a therapeutically effective amount of a compound of general formula (I) to a subject in need thereof.

Another aspect of the invention is a pharmaceutical composition, which comprises at least a compound of general formula (I) or a pharmaceutically acceptable salt, co-crystal, prodrug, isomer or solvate thereof, and at least a pharmaceutically acceptable carrier, additive, adjuvant or vehicle.

The pharmaceutical composition of the invention can be formulated as a medicament in different pharmaceutical forms comprising at least a compound of formula (I) binding to the subunit α2δ, especially α2δ-1 subunit of voltage-gated calcium channels and noradrenaline transporter (NET) and optionally at least one further active substance and/or optionally at least one auxiliary substance.

The auxiliary substances or additives can be selected among carriers, excipients, support materials, lubricants, fillers, solvents, diluents, colorants, flavour conditioners such as sugars, antioxidants and/or agglutinants. In the case of suppositories, this may imply waxes or fatty acid esters or preservatives, emulsifiers and/or carriers for parenteral application. The selection of these auxiliary materials and/or additives and the amounts to be used will depend on the form of application of the pharmaceutical composition.

The pharmaceutical composition in accordance with the invention can be adapted to any form of administration, be it orally or parenterally, for example pulmonarily, nasally, rectally and/or intravenously.

Preferably, the composition is suitable for oral or parenteral administration, more preferably for oral, intravenous, intraperitoneal, intramuscular, subcutaneous, intrathecal, rectal, transdermal, transmucosal or nasal administration.

The composition of the invention can be formulated for oral administration in any form preferably selected from the group consisting of tablets, drageés, capsules, pills, chewing gums, powders, drops, gels, juices, syrups, solutions and suspensions. The composition of the present invention for oral administration may also be in the form of multiparticulates, preferably microparticles, microtablets, pellets or granules, optionally compressed into a tablet, filled into a capsule or suspended in a suitable liquid. Suitable liquids are known to those skilled in the art.

The compounds of the invention can be formulated as deposits in dissolved form or in patches, for percutaneous application.

Skin applications include ointments, gels, creams, lotions, suspensions or emulsions.

The preferred form of rectal application is by means of suppositories.

In a preferred embodiment, the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

The following examples are merely illustrative of certain embodiments of the invention and cannot be considered as restricting it in any way.

### EXAMPLES

In the next preparation examples, the preparation of both intermediate compounds as well as compounds according to the invention are disclosed.

### Examples

The following abbreviations are used in the examples:

ACN: acetonitrile

CH: cyclohexane

DCM: dichloromethane

DIPEA: N,N-diisopropylethylamine

DMA: N,N-dimethylacetamide

DME: 1,2-dimethoxyethane

DMF: N,N-dimethylformamide

dppf: 1,1′-ferrocenediyl-bis(diphenylphosphine)

Et₂O: diethyl ether

EtOAc; ethyl acetate

EtOH: ethanol

EX: example

h: hour/s

HATU: O-(7-azabenzotriazol-1-yl)-N,N,N′,N-tetramethyluronium hexafluorophosphate

HPLC: high performance liquid chromatography

LDA: lithium diisopropylamide

MeOH: methanol

MS: mass spectrometry

Min.: minutes

Quant: quantitative

Ret.: retention

r.t.: room temperature

Sat: saturated

Sol.: solution

SPhos: 2-dicyclohexylphosphino-2′,6′-dimethoxybiphenyl

TEA: triethylamine

THF: tetrahydrofuran

Wt: weight

The following methods were used to determine the HPLC-MS spectra:

Method A

Column Xbridge C18 XP 30×4.6 mm, 2.5 um

Temperature: 40° C.

Flow: 2.0 mL/min

Gradient: NH₄HCO₃ pH 8:ACN (95:5)—0.5 min—(95:5)—6.5 min—(0:100)—1 min—(0:100)

Sample dissolved approx. 1 mg/mL in NH₄HCO₃ pH 8/ACN

Method B

Column: Gemini-NX 30×4.6 mm, 3 um

Temperature: 40° C.

Flow: 2.0 mL/min

Gradient: NH₄HCO₃ pH 8: ACN (95:5)—0.5 min—(95:5)—6.5 min—(0:100)—1 min—(0:100)

Sample dissolved approx. 1 mg/mL in NH₄HCO₃ pH 8/ACN

Method C

Column: Kinetex EVO 50×4.6 mm, 2.6 um

Temperature: 40° C.

Flow: 2.0 mL/min

Gradient: NH₄HCO₃ pH 8: ACN (95:5)—0.5 min—(95:5)—6.5 min—(0:100)—1 min—(0:100)

Sample dissolved approx. 1 mg/mL in NH₄HCO₃ pH 8/1 CAN

Method D

Column: Kinetex EVO 50×4.6 mm, 2.6 um

Temperature: 40° C.

Flow: 1.5 mL/min

Gradient: NH₄HCO₃ pH 8: ACN (95:5)—0.5 min—(95:5)—6.5 min—(0:100)—1 min—(0:100)

Sample dissolved approx. 1 mg/mL in NH₄HCO₃ pH 8/ACN

Method E

Column: Kinetex EVO 50×4.6 mm, 2.6 um

Temperature: 40° C.

Flow: 1.5 mL/min

Gradient: NH₄HCO₃ pH 8: ACN (95:5)—0.5 min—(95:5)—6.5 min—(0:100)—2 min—(0:100)

Sample dissolved approx. 1 mg/mL in NH₄HCO₃ pH 8/ACN

Method F

Column: Gemini C18 30×4.6 mm 3 um

Temperature: 40° C.

Flow: 1.5 mL/min

Gradient H2O—0.1% HCOOH/ACN (95:5)—0.5 min—(95:5)—8.5 min—(0:100)—1 min—(0:100)

Sample dissolved approx. 1 mg/mL in ACN

### Synthesis of Intermediates

### Intermediate 1A: 6-Methoxy-2,3-dihydro-1H-pyrrolo[3,2-b]pyridine

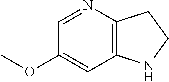

### Step 1. Tert-Butyl 6-methoxy-1H-pyrrolo[3,2-b]pyridine-1-carboxylate

A solution of 6-methoxy-1H-pyrrolo[3,2-b]pyridine (0.45 g, 3.0 mmol) in DCM (6 mL) was cooled at 0° C. Then, TEA (0.63 mL, 4.5 mmol) and a solution of di-tert-butyl dicarbonate (0.73 g, 3.3 mmol) in DCM (6 mL) were sequentially added and the mixture was stirred at r.t. overnight. Water was added, the layers were separated and the aqueous phase was back extracted with DCM. The combined organic phases were washed with brine, dried over MgSO₄ and concentrated under vacuum. The residue was purified by flash chromatography, silica gel, gradient CH/EtOAc 100:0 to CH/EtOAc 0:100 to give the title compound (627 mg, 83% yield).

### Step 2. tert-Butyl 6-methoxy-2,3-dihydro-1H-pyrrolo[3,2-b]pyridine-1-carboxylate

A mixture of the product obtained in Step 1 (627 mg, 2.52 mmol) and palladium hydroxide (180 mg, 20% wt on carbon, wet) in EtOH (90 mL) was stirred under 2 bars of H₂ at 50° C. for 1 day. The catalyst was filtered off and the solvent was removed under vacuum to give the title compound as a crude product that was used as such without further purification (590 mg, 93% yield).

### Step 3. Title Compound

HCl (2.5 mL, 4 M solution in 1,4-dioxane, 10 mmol) was carefully added to a solution of the product obtained in Step 2 (590 mg, 2.36 mmol) in a mixture of MeOH (2.8 mL) and 1,4-dioxane (0.7 mL) and the mixture was stirred at r.t. overnight. It was then concentrated to dryness and the residue was dissolved in water. The pH was made basic with 1 M NaOH solution and it was extracted with DCM. The combined organic phases were dried over MgSO₄ and concentrated under vacuum to yield the title compound (187 mg, 53% yield).

This method was used for the preparation of Intermediates 1B-1C using suitable starting materials:

| INT | Structure | Chemical name |
|---|---|---|
| 1B | 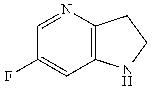 | 6-Fluoro-2,3-dihydro- 1H-pyrrolo[3,2- b]pyridine |
| 1C | 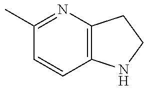 | 5-Methyl-2,3-dihydro- 1H-pyrrolo[3,2- b]pyridine |

### Intermediate 2A: 5-Isopropyl-2,3-dihydro-1H-pyrrolo[3,2-b]pyridine

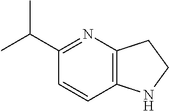

### Step 1. 5-(Prop-1-en-2-yl)-1H-pyrrolo[3,2-b]pyridine

A mixture of 5-chloro-1H-pyrrolo[3,2-b]pyridine (1.0 g, 6.6 mmol), 4,4,5,5-tetramethyl-2-(prop-1-en-2-yl)-1,3,2-dioxaborolane (1.21 g, 7.2 mmol), K₂CO₃ (2.72 g, 19.7 mmol) and dichloro 1,1′-bis(diphenylphosphino)ferrocene palladium(II) dichloromethane adduct (0.48 g, 0.66 mmol) in a mixture of 1,4-dioxane (15 mL) and water (5 mL) was heated in a sealed tube under an argon atmosphere at 120° C. overnight. After cooling, the solids were filtered off and the filtrate was concentrated to dryness. The residue was purified by flash chromatography, silica gel, gradient DCM to MeOH:DCM (1:4) to give the title compound (overweight, quant. yield assumed).

### Step 2. tert-Butyl 5-(prop-1-en-2-yl)-1H-pyrrolo[3,2-b]pyridine-1-carboxylate

Following the protection procedure described for the preparation of Step 1 of Intermediate 1A using the product obtained in Step 1 as starting material, the title compound was obtained (1.38 g, 90% yield).

### Step 3. tert-Butyl 5-isopropyl-2,3-dihydro-1H-pyrrolo[3,2-b]pyridine-1-carboxylate

Following the hydrogenation procedure described for the preparation of Step 2 of Intermediate 1A using the product obtained in Step 2 as starting material, the title compound was obtained (744 mg, 53% yield).

### Step 4. Title Compound

Following the deprotection procedure described for the preparation of Step 3 of Intermediate 1A using the product obtained in Step 3 as starting material, the title compound was obtained (388 mg, 81% yield).

This method was used for the preparation of Intermediates 2B-2D using suitable starting materials:

| INT | Structure | Chemical name |
|---|---|---|
| 2B | 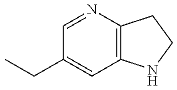 | 6-Ethyl-2,3-dihydro- 1H-pyrrolo[3,2- b]pyridine |
| 2C | 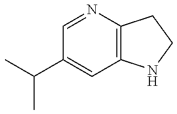 | 6-Isopropyl-2,3- dihydro-1H- pyrrolo[3,2-b]pyridine |
| 2D | 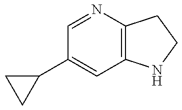 [SMILES: c1nc2c(cc1C1CC1)NCC2] | 6-Cyclopropyl-2,3- dihydro-1H- pyrrolo[3,2- b]pyridine⁽¹⁾ |

⁽¹⁾Cs₂CO₃ was used as base and a mixture of THF-water 9:1 was used as solvent.

### Intermediate 3A: 3,3,5-Trimethyl-2,3-dihydro-1H-pyrrolo[3,2-b]pyridine

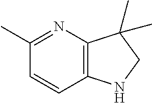

### Step 1. 6-Chloro-2-iodo-N-(2-methylallyl)pyridin-3-amine

Potassium tert-butoxide (0.79 g, 7.1 mmol) was added to a solution of 6-chloro-2-iodopyridin-3-amine (1.5 g, 5.9 mmol) in dry THF (34 mL) and the mixture was stirred at r.t. for 15 min. Then, 3-bromo-2-methyl-1-propene (0.73 mL, 7.1 mmol) was slowly added and the reaction mixture was stirred at r.t. for 2.5 days. Then, it was concentrated to dryness and the residue was diluted with water and DCM. The layers were separated and the aqueous phase was back extracted with DCM. The combined organic phases were dried over MgSO₄ and concentrated under vacuum. The residue was purified by flash chromatography, silica gel, gradient CH/EtOAc 100:0 to CH/EtOAc 0:100 to give the title compound (1.31 g, 72% yield).

### Step 2. 5-Chloro-3,3-dimethyl-2,3-dihydro-1H-pyrrolo[3,2-b]pyridine

A mixture of the product obtained in Step 1 (1.31 g, 4.25 mmol), tetrabutylammonium chloride (1.4 g, 5.1 mmol), TEA (1.77 mL, 12.7 mmol) and sodium formate (0.35 g, 5.1 mmol) in a mixture of DMSO (30 mL) and water (1.3 mL) was degassed by bubbling nitrogen gas through the mixture. Palladium(II) acetate (0.143 g, 0.64 mmol) was added and the mixture was heated at 120° C. for 1 h under a nitrogen atmosphere. After cooling, the solids were filtered off and the filtrate was diluted with water and EtOAc. The phases were separated and the aqueous phase was back extracted with EtOAc (×3). The combined organic phases were washed with water (×4), dried over MgSO₄ and concentrated concentrated to dryness. The residue was purified by flash chromatography, silica gel, gradient CH/EtOAc 100:0 to CH/EtOAc 0:100 to give the title compound (450 mg, 58% yield). Step 3. Title compound: A mixture of the product obtained in Step 2 (0.45 g, 2.46 mmol), trimethylboroxine (0.31 g, 2.46 mmol), K₂CO₃ (1.02 g, 7.39 mmol) and dichloro 1,1′-bis(diphenylphosphino)ferrocene palladium(II) dichloromethane adduct (9.9 mg, 0.135 mmol) in DME (15 mL) was placed in a microwave vial. The system was purged with vacuum/argon cycles and it was irradiated under microwave heating at 120° C. for 1 h. After cooling, the solids were filtered off and the filtrate was concentrated to dryness. The residue was purified by flash chromatography, silica gel, gradient DCM to MeOH:DCM (1:4) to give the title compound (294 mg, 73% yield).

This method was used for the preparation of Intermediates 3B-3C using suitable starting materials:

| INT | Structure | Chemical name |
|---|---|---|
| 3B | 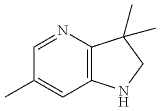 | 3,3,6-Trimethyl-2,3- dihydro-1H- pyrrolo[3,2- b]pyridine⁽¹⁾ |
| 3C | 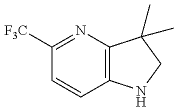 | 3,3-Dimethyl-5- (trifluoromethyl)-2,3- dihydro-1H- pyrrolo[3,2- b]pyridine⁽²⁾ |

⁽¹⁾Conventional thermal heating at 120° C. overnight was used instead of microwave heating.
⁽²⁾Step 3 was not performed.

### Intermediate 4: (E)-Ethyl 3-(5-fluorothiophen-2-yl)acrylate

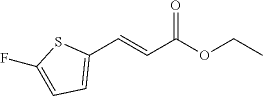

Ethyl 2-(triphenylphosphoranylidene)acetate (1.09 g, 3.13 mmol) was added to a solution of 5-fluorothiophene-2-carbaldehyde (0.41 g, 3.13 mmol) in dry toluene (6.2 mL) and the mixture was heated to reflux under a nitrogen atmosphere for 7 h. Then, it was allowed to cool down to r.t. Et₂O (10 mL) was added and the resulting suspension was stirred at r.t. for 1 h. The precipitated solids were filtered off and discarded, and the filtrate was concentrated to dryness. The residue was purified by flash chromatography, silica gel, gradient DCM to MeOH:DCM (1:4) to give the title compound (435 mg, 69% yield).

### Intermediate 5: (E)-3-(4-Methylthiophen-3-yl)acrylic Acid

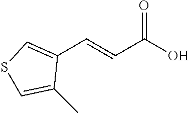

### Step 1. (E)-Ethyl 3-(4-bromothiophen-3-yl)acrylate

Following the procedure described for the preparation of Intermediate 4 but using 4-bromothiophene-3-carbaldehyde as starting material, the title compound was obtained (831 mg, 61% yield). Step 2. (E)-Ethyl 3-(4-methylthiophen-3-yl)acrylate: Starting from the product obtained in Step 1 and following the experimental procedure described in Step 3 of Intermediate 3A, the title compound was obtained (410 mg, 66% yield).

### Step 3. Title Compound

1 M NaOH (12 mL) was added to a solution of the product obtained in Step 2 (410 mg, 2.09 mmol) in THF (15 mL) and the mixture was stirred at r.t. for 2 days. Then it was poured over 1 M HCl and it was extracted with EtOAc (×3). The combined organic phases were dried over MgSO₄ and concentrated to dryness to give the title compound (342 mg, 97% yield).

### Intermediate 6: 2-(1,3-Dichloropropyl)thiophene

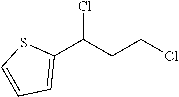

To a solution of 3-chloro-1-(thiophen-2-yl)propan-1-ol (1.0 g, 5.66 mmol) in DCM (34 mL), cooled at 0° C., TEA (1.02 mL, 7.36 mmol) and methanesulfonyl chloride (0.48 mL, 6.23 mmol) were added dropwise and the mixture was stirred at 0° C. overnight. Ice was added and then it was diluted with NaHCO₃ sat. sol. and DCM. The phases were separated and the aqueous phase was back extracted with DCM. The combined organic phases were washed with brine, dried over MgSO₄ and concentrated to dryness to give the title compound (1.04 g, 94% yield).

### Intermediate 7: 6-Fluoro-3,3-dimethyl-2,3-dihydro-1H-pyrrolo[3,2-b]pyridine

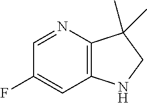

### Step 1. 2-(3,5-Difluoropyridin-2-yl)-2-methylpropanenitrile

To a solution of 2,3,5-trifluoropyridine (8 g, 60.1 mmol) and isobutyronitrile (10.8 mL, 120 mmol) in toluene (20 mL), cooled at 0° C., sodium bis(trimethylsilylamide) solution (31.6 mL, 1.9 M in THF, 60.1 mmol) was added dropwise and the reaction mixture was stirred at r.t. overnight. It was concentrated to dryness and re-dissolved in EtOAc. The organic phase was washed with NH₄Cl sat. sol., water and brine. dried over MgSO₄ and concentrated to dryness. The residue was purified by flash chromatography, silica gel, gradient CH/EtOAc 100:0 to 0:100 to give the title compound (4.5 g, 41% yield).

### Step 2. 2-(3,5-Difluoropyridin-2-yl)-2-methylpropan-1-amine

To a solution of the product obtained in Step 1 (4.5 g, 25.03 mmol) in MeOH (100 mL), cooled at 0° C., cobalt(II) chloride hexahydrate (2.98 g, 12.52 mmol) was added, followed by sodium borohydride (4.74 g, 125 mmol) and the reaction mixture was stirred at r.t. overnight. Then, it was cooled to 0° C. and conc. ammonia (40 mL) was slowly added. The mixture was stirred at 0° C. for 30 min. and it was filtered over a pad of Celite that was washed with MeOH. The filtrate was evaporated and the residue thus obtained was diluted with water and conc. ammonia. The aqueous phase was extracted with EtOAc and the combined organic extracts were washed with water and brine, dried over MgSO₄ and concentrated to dryness to give the title compound (3.6 g, 77% yield).

### Step 3. Title Compound

In 3 separate microwave vials, the product obtained in Step 2 (1.2 g, 6.4 mmol, each vial) and K₂CO₃ (4 g, 28.9 mmol, each vial) were suspended in DMSO (8 mL, each vial). The reaction was irradiated under microwave heating at 150° C. for 40 min. The reaction mixtures were combined, poured onto water and extracted with EtOAc. The combined organic extracts were washed with water and brine, dried over MgSO₄ and concentrated to dryness. The crude compound was purified by flash chromatography, silica gel, gradient CH/EtOAc 100:0 to 0:100 to give the title compound (1.35 g, 42% yield).

### Intermediate 8: 6-Fluoro-3,3,5-trimethyl-2,3-dihydro-1H-pyrrolo[3,2-b]pyridine

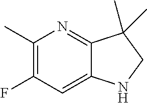

### Step 1. 5-Bromo-6-fluoro-3,3-dimethyl-2,3-dihydro-1H-pyrrolo[3,2-b]pyridine

To a solution of Intermediate 7 (1.4 g, 8.75 mmol) in ACN (50 mL), cooled at 0° C., N-bromosuccinimide (779 mg, 4.38 mmol) was added portionwise. The reaction was stirred at 0° C. for 1 h. Then it was diluted with EtOAc and the organic phase was washed with brine, dried over MgSO₄ and concentrated to dryness to give the title compound as a crude product (1.56 g, 74% yield). 1.2 g of the crude product were purified by flash chromatography, silica gel, gradient CH/EtOAc 100:0 to 0:100 to give the title compound in higher purity (0.7 g, 42% yield)

### Step 2. Title Compound

In a microwave vial, the product obtained in Step 1 (688 mg, 2.81 mmol), K₂CO₃ (2.5 g, 18.2 mmol), trimethylboroxine (0.43 mL, 3.09 mmol) and dichloro 1,1′bis(diphenylphosphino)ferrocenepalladium(II) dichloromethane adduct (458 mg, 0.56 mmol) were suspended in DME (15 mL) under a N₂ atmosphere. The reaction was irradiated under microwave heating at 120° C. for 1 h. The mixture was filtered through a pad of Celite that was washed with EtOAc. The solvent was evaporated and the residue was dissolved in EtOAc. The organic phase was washed with water and brine, dried over MgSO₄ and concentrated to dryness. The residue was purified by flash chromatography, silica gel, gradient CH/EtOAc 100:0 to 0:100 to give the title compound (258 mg, 51% yield).

### Intermediate 9: 3,3-Dimethyl-2,3-dihydro-1H-pyrrolo[3,2-b]pyridine-6-carbonitrile

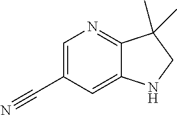

A mixture of the product obtained in Step 2 of Intermediate 3B (428 mg, 1.88 mmol), SPhos (77 mg, 0.188 mmol), tris(dibenzylideneacetone)dipalladium(0) (86 mg, 0.094 mmol) and zinc cyanide (332 mg, 2.83 mmol) in DMF (7.5 mL) was placed in a microwave vial. The system was inertized with argon and it was irradiated under microwave heating at 150° C. for 35 min. After cooling, aq. NH₄Cl sat. sol. and EtOAc were added. The phases were separated and the aqueous phase was extracted with EtOAc. The combined organic phases were dried over MgSO₄ and concentrated to dryness. The residue was purified by flash chromatography, silica gel, gradient CH/EtOAc 100:0 to 0:100 to give the title compound (228 mg, 70% yield).

### Intermediate 10: 3,3-Diethyl-2,3-dihydro-1H-pyrrolo[3,2-b]pyridine

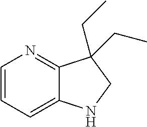

### Step 1. Diethyl 2-(3-nitropyridin-2-yl)malonate

NaH (11.7 g, 294 mmol, 60 wt % dispersion in mineral oil) was washed with heptane (3×120 mL) and dried under a N₂ stream. To a suspension of the purified NaH in DMSO (160 mL), diethyl malonate (47.1 g, 294 mmol) was added. After stirring for 30 min at r.t., 2-chloro-3-nitropyridine (20 g, 126 mmol) was added in one portion and the reaction mixture was heated at 100° C. for 15 min. After cooling down to r.t., the reaction mixture was poured onto NH₄Cl sat. sol. and it was extracted with EtOAc/CH 50:50. The organic phase was dried over MgSO₄ and concentrated to dryness to afford the title compound (73 g, overweight, quant. yield assumed).

### Step 2. Ethyl 2-(3-nitropyridin-2-yl)acetate

To a solution of the product obtained in Step 1 (35 g, 49 wt %, 60.8 mmol) in DMSO (220 mL), LiCl (7.73 g, 182 mmol) and water (0.8 mL) were added. The mixture was stirred at 110° C. overnight. Additional LiCl (3.86 g, 91 mmol) and water (0.4 mL) were added and the mixture was heated again at 110° C. overnight. Then, NH₄Cl sat. sol. and EtOAc were added, the phases were separated and the aqueous phase was extracted with EtOAc. The combined organic extracts were washed with brine, dried over MgSO₄ and concentrated to dryness. The residue was purified by flash chromatography, silica gel, gradient CH/EtOAc 100:0 to 0:100 to give the title compound (7.73 g, 60% yield).

### Step 3. Ethyl 2-ethyl-2-(3-nitropyridin-2-yl)butanoate

To a solution of the product obtained in Step 2 (2.0 g, 9.52 mmol) in DMF (28 mL), cooled at 0° C. under a N₂ atmosphere, NaH (419 mg, 10.47 mmol, 60 wt % dispersion in mineral oil) was added. After stirring for 30 min. at 0° C., iodoethane (0.84 mL, 10.47 mmol) was added and the reaction mixture was stirred at r.t. for 4 h. Then, the reaction mixture was again cooled to 0° C. and additional NaH (419 mg, 10.47 mmol) was added. After stirring for 30 min. at 0° C., additional iodoethane (0.84 mL, 10.47 mmol) was added and the mixture was stirred at r.t. overnight. Water was added and it was extracted with EtOAc. The combined organic phases were dried over MgSO₄ and concentrated to dryness to afford a crude compound that was purified by flash chromatography, silica gel, gradient CH/EtOAc 100:0 to 0:100 to give the title compound (1.7 g, 66% yield).

### Step 4. 3,3-Diethyl-1H-pyrrolo[3,2-b]pyridin-2(3H)-one

A suspension of the product obtained in Step 3 (1.7 g, 6.34 mmol) and iron (2.4 g, 43.1 mmol) in acetic acid was heated at 100° C. for 2 h. After cooling down to r.t., the mixture was filtered through a pad of Celite, that was washed with EtOAc and the filtrate was concentrated to dryness. The crude product was purified by flash chromatography, silica gel, gradient DCM to MeOH:DCM (1:4) to give the title compound (0.684 g, 57% yield).

### Step 5. Title Compound

To a solution of the product obtained in Step 4 (411 mg, 2.16 mmol) in THF (43 mL), cooled at 0° C., NaBH₄ (409 mg, 10.80 mmol) was added, followed by boron trifluoride diethyl etherate (3.97 mL, 15.12 mmol) and the mixture was stirred at r.t. overnight. Then, it was again cooled to 0° C. and additional NaBH₄ (204 mg, 5.40 mmol) and boron trifluoride diethyl etherate (2 mL, 7.56 mmol) were added. The reaction mixture was stirred at r.t. for an additional day. NH₄Cl sat. sol. (45 mL) and water (140 mL) were added, the pH of the mixture was adjusted to 9 with 6 N NaOH aq. sol. and it was extracted with EtOAc. The combined organic fractions were dried over MgSO₄ and concentrated to dryness to afford a crude compound that was purified by flash chromatography, silica gel, gradient DCM to MeOH:DCM (1:4) to give the title compound (178 mg, 47% yield).

### Intermediate 11: 5-Methoxy-3,3-dimethyl-2,3-dihydro-1H-pyrrolo[3,2-b]pyridine

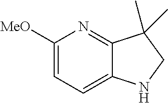

To a solution of the product obtained in Step 2 of Intermediate 3A (487 mg, 2.67 mmol) in DMF (10.6 mL), sodium methoxide solution (6.1 mL, 25 wt % in MeOH, 26.7 mmol) and copper(I) bromide (765 mg, 5.33 mmol) were added. The mixture was heated at 140° C. for 2 h in a sealed tube. After cooling down to r.t., water and NaHCO₃ sat. sol. were added, and the aqueous phase was extracted with EtOAc. The combined organic phases were dried over MgSO₄ and concentrated to dryness. The residue was purified by flash chromatography, silica gel, gradient CH/EtOAc 100:0 to 0:100 to give the title compound (218 mg, 46% yield).

### Intermediate 12: 3,3-Dimethylindoline-4-carbonitrile

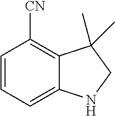

### Step 1. 1-Acetyl-4-bromoindolin-2-one

A solution of 4-bromoindolin-2-one (1.12 g, 5.32 mmol) and acetic anhydride (1.3 mL, 13.83 mmol) in xylene (12 mL) was heated at reflux for 3 days. Additional acetic anhydride (0.5 mL, 5.32 mmol) was added after 24 h and 48 h of reaction. Then, the mixture was concentrated to dryness and the residue was dissolved in EtOAc. The organic phase was washed with NaHCO₃ sat. sol. dried over MgSO₄ and concentrated to dryness. The crude product was purified by flash chromatography, silica gel, gradient CH/EtOAc 100:0 to 0:100 to give the title compound (522 mg, 39% yield).

### Step 2. 1-Acetyl-4-bromo-3,3-dimethylindolin-2-one

Following the experimental procedure described for the preparation of Step 3 of Intermediate 10, using the product obtained in Step 1 and iodomethane as staring materials, the title compound was obtained (488 mg, 36% yield).

### Step 3. 4-Bromo-3,3-dimethylindolin-2-one

To a solution of the product obtained in Step 2 (488 mg, 1.73 mmol) in EtOH (7.2 mL), 3 M NaOH aq. sol. (0.29 mL, 0.865 mmol) was added and the mixture was stirred at r.t. for 2 h. NH₄Cl sat. sol. was added and the aqueous phase was extracted with EtOAc. The combined organic fractions were dried over MgSO₄ and concentrated to dryness to afford the title compound (408 mg, 98% yield).

### Step 4. 4-Bromo-3,3-dimethylindoline

Following the experimental procedure described for the preparation of Step 5 of Intermediate 10, starting from the product obtained in Step 4, the title compound was obtained (190 mg, 49% yield).

### Step 5. Title Compound

A mixture of the product obtained in Step 4 (190 mg, 0.84 mmol), dppf (94 mg, 0.168 mmol), tris(dibenzylideneacetone)dipalladium(0) (77 mg, 0.084 mmol) and zinc cyanide (11 mg, 0.168 mmol) in DMA (4 mL) was placed in a microwave vial. The system was inertized with argon and it was irradiated under microwave heating at 150° C. for 30 min. After cooling down, water and EtOAc were added, the phases were separated and the aqueous phase was extracted with EtOAc. The combined organic phases were dried over MgSO₄ and concentrated to dryness. The residue was purified by flash chromatography, silica gel, gradient CH/EtOAc 100:0 to CH/EtOAc 0:100 to give the title compound (47 mg, 32% yield).

### Intermediate 13A. (E)-4-Methoxyphenyl 3-(thiophen-2-yl)acrylate

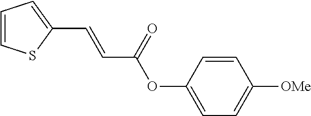 [SMILES: COc1ccc(OC(=O)/C=C/c2cccs2)cc1]

To a solution of (E)-3-(thiophen-2-yl)acryloyl chloride (1.12 g, 6.49 mmol) and 4-methoxyphenol (1.2 g, 9.73 mmol) in DCM (6.8 mL), cooled at 0° C., TEA (1.8 mL, 12.98 mmol) was added and the reaction was stirred at r.t. overnight. Water was added, the phases were separated and the aqueous phase was extracted with DCM. The combined organic phases were dried over MgSO₄ and concentrated to dryness. The residue was purified by flash chromatography, silica gel, gradient CH/EtOAc 100:0 to CH/EtOAc 0:100 to give the title compound (1.37 g, 81% yield).

This method was used for the preparation of Intermediate 13B using suitable starting materials:

| INT | Structure | Chemical name |
|---|---|---|
| 13B | 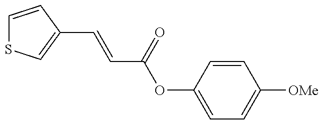 [SMILES: COc1ccc(OC(=O)/C=C/c2ccsc2)cc1] | (E)-4-Methoxyphenyl 3-(thiophen-3- yl)acrylate |

### Synthesis of Examples

### Example 1: 3-(Indolin-1-yl)-N-methyl-3-(thiophen-2-yl)propan-1-amine

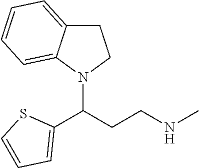 [SMILES: CNCCC(c1cccs1)N1CCc2ccccc21]

### Step 1. 1-(3-Chloro-1-(thiophen-2-yl)propyl)indoline

To a solution of indoline (92 mg, 0.77 mmol) in ACN (0.5 mL), K₂CO₃ (53 mg, 0.38 mmol) was added and the mixture was stirred at r.t. for 30 min. Then, a solution of Intermediate 6 (50 mg, 0.26 mmol) in ACN (0.5 mL) was added dropwise and the mixture was heated at 70° C. overnight. It was then allowed to cool, and it was diluted with ammonium chloride sat. sol and EtOAc. The phases were separated and the aqueous phase was extracted with EtOAc. The combined organic phases were washed with brine, dried over MgSO₄ and concentrated to dryness. The crude product was purified by flash chromatography, silica gel, gradient CH/EtOAc 100:0 to CH/EtOAc 50:50 to give the title compound (34 mg, 47% yield).

### Step 2. Title Compound

In a sealed tube, a solution of the product obtained in Step 1 (34 mg, 0.12 mmol) and methylamine (33 wt % in EtOH, 1 mL, 8.1 mmol) was heated at 90° C. for 2 days. Then, the solvent was concentrated. The crude product was purified by flash chromatography, silica gel, gradient DCM to MeOH:DCM (1:4) to give the title compound (8 mg, 24% yield).

HPLC retention time (method A): 3.55 min; MS: 273.1 (M+H).

### Example 2: 3-(2,3-Dihydro-1H-pyrrolo[2,3-c]pyridin-1-yl)-N-methyl-3-(thiophen-2-yl)propan-1-amine

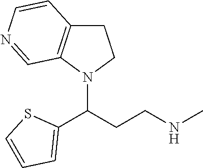 [SMILES: CNCCC(c1cccs1)N1CCc2ccncc21]

### Step 1. Ethyl 3-(2,3-dihydro-1H-pyrrolo[3,2-c]pyridin-1-yl)-3-(thiophen-2-yl)propanoate

To a solution of 2,3-dihydro-1H-pyrrolo[2,3-c]pyridine (157 mg, 1.31 mmol) in dry THF (4 mL), cooled at −78° C., LDA solution (1.5 M in THF/ethylbenzene/heptane, 1 mL, 1.5 mmol) was added dropwise and the mixture was stirred at −78° C. for 30 min. Then, a solution of (E)-ethyl 3-(thiophen-2-yl)acrylate (216 mg, 1.19 mmol) in dry THF (4 mL) was slowly added and the reaction mixture was stirred at −78° C. for 1.5 h. Aqueous NH₄Cl sat. sol. and EtOAc were added, the phases were separated and the aqueous phase was extracted with EtOAc. The combined organic phases were washed with brine, dried over MgSO₄ and concentrated to dryness. The crude product was purified by flash chromatography, silica gel, gradient CH/EtOAc 100:0 to CH/EtOAc 0:100 to give the title compound (153 mg, 42% yield).

### Step 2. 3-(2,3-Dihydro-1H-pyrrolo[3,2-c]pyridin-1-yl)-N-methyl-3-(thiophen-2-yl)propanamide

In a sealed tube, a solution of the product obtained in Step 1 (153 mg, 0.51 mmol) and methylamine (33 wt % in EtOH, 1.25 mL, 10.1 mmol) was heated at 100° C. overnight. Then, the solvent was concentrated to dryness to give the title compound as a crude product that was directly used in the following step (145 mg, quant. yield). Step 3. Title compound: To a solution of the product obtained in Step 2 (145 mg, 0.51 mmol) in THF (4 mL), borane-methyl sulfide complex (0.24 mL, 2.52 mmol) was added at r.t. The reaction mixture was heated to reflux for 4 h, then it was cooled to r.t. and it was concentrated to dryness. The residue was dissolved in MeOH (10 mL), 1 M HCl (5 mL) was added and the resulting mixture was heated to reflux for 1 h and then it was stirred at r.t. overnight. It was concentrated to dryness and the residue was diluted with DCM and 1 M NaOH. The phases were separated and the aqueous phase was back extracted with DCM. The organic phases were combined, dried over MgSO₄, filtered and concentrated to dryness. The crude product was purified by flash chromatography, silica gel, gradient DCM to MeOH:DCM (1:4) to give the title compound (63 mg, 45% yield).

HPLC retention time (method B): 2.48 min; MS: 274.1 (M+H).

This method was used for the preparation of Examples 3-14 using suitable starting materials:

| | | | | Ret | |
|---|---|---|---|---|---|
| | | | HPLC | time | MS |
| EX | Structure | Chemical name | Method | (min) | (M + H) |
| 3 | 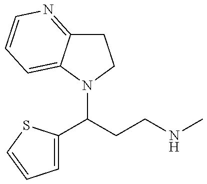 [SMILES: CNCCC(c1cccs1)N1CCc2ncccc21] | 3-(2,3-Dihydro-1H- pyrrolo[3,2-b]pyridin-1- yl)-N-methyl-3- (thiophen-2-yl)propan-1- amine | B | 2.39 | 274.1 |
| 4 | 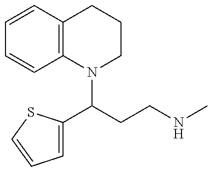 [SMILES: CNCCC(c1cccs1)N1CCCc2ccccc21] | 3-(3,4-Dihydroquinolin- 1(2H)-yl)-N-methyl-3- (thiophen-2-yl)propan-1- amine | B | 4.08 | 287.1 |
| 5 | 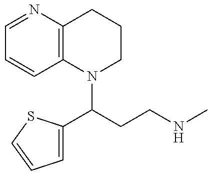 [SMILES: CNCCC(c1cccs1)N1CCCc2ncccc21] | 3-(3,4-Dihydro-1,5- naphthyridin-1(2H)-yl)- N-methyl-3-(thiophen-2- yl)propan-1-amine | C | 2.71 | 288.1 |
| 6 | 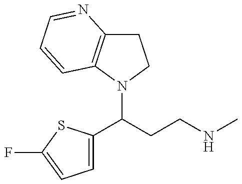 [SMILES: CNCCC(c1ccc(F)s1)N1CCc2ncccc21] | 3-(2,3-Dihydro-1H- pyrrolo[3,2-b]pyridin-1- yl)-3-(5-fluorothiophen- 2-yl)-N-methylpropan-1- amine | C | 2.76 | 292.1 |
| 7 | 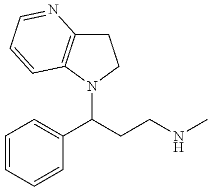 [SMILES: CNCCC(c1ccccc1)N1CCc2ncccc21] | 3-(2,3-Dihydro-1H- pyrrolo[3,2-b]pyridin-1- yl)-N-methyl-3- phenylpropan-1-amine | C | 2.75 | 268.1 |
| 8 | 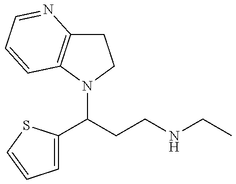 [SMILES: CCNCCC(c1cccs1)N1CCc2ncccc21] | 3-(2,3-Dihydro-1H- pyrrolo[3,2-b]pyridin-1- yl)-N-ethyl-3-(thiophen- 2-yl)propan-1-amine⁽¹⁾ | D | 3.16 | 288.0 |
| 9 | 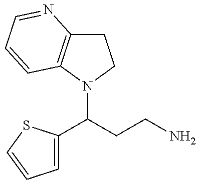 [SMILES: NCCC(c1cccs1)N1CCc2ncccc21] | 3-(2,3-Dihydro-1H- pyrrolo[3,2-b]pyridin-1- yl)-3-(thiophen-2- yl)propan-1-amine⁽²⁾ | E | 2.98 | 260.0 |
| 10 | 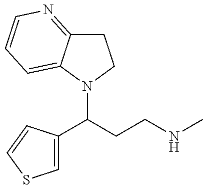 [SMILES: CNCCC(c1ccsc1)N1CCc2ncccc21] | 3-(2,3-Dihydro-1H- pyrrolo[3,2-b]pyridin-1- yl)-N-methyl-3- (thiophen-3-yl)propan-1- amine | E | 3.04 | 274.0 |
| 11 | 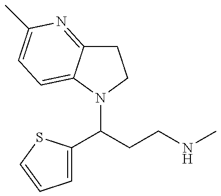 | N-methyl-3-(5-methyl- 2,3-dihydro-1H- pyrrolo[3,2-b]pyridin-1- yl)-3-(thiophen-2- yl)propan-1-amine | E | 3.20 | 288.0 |
| 12 | 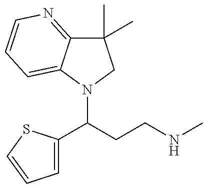 [SMILES: CNCCC(c1cccs1)N1CC(C)(C)c2ncccc21] | 3-(3,3-Dimethyl-2,3- dihydro-1H-pyrrolo[3,2- b]pyridin-1-yl)-N-methyl- 3-(thiophen-2-yl)propan- 1-amine | E | 3.44 | 302.0 |
| 13 | 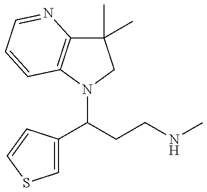 [SMILES: CNCCC(c1ccsc1)N1CC(C)(C)c2ncccc21] | 3-(3,3-Dimethyl-2,3- dihydro-1H-pyrrolo[3,2- b]pyridin-1-yl)-N-methyl- 3-(thiophen-3-yl)propan- 1-amine | E | 3.52 | 302.1 |
| 14 | 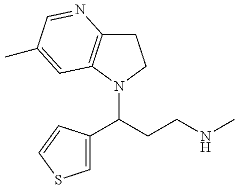 | N-methyl-3-(6-methyl- 2,3-dihydro-1H- pyrrolo[3,2-b]pyridin-1- yl)-3-(thiophen-3- yl)propan-1-amine | E | 3.42 | 288.1 |

⁽¹⁾Ethylamine solution was used in Step 2 instead of methylamine
⁽²⁾Ammonia was used in Step 2 instead of methylamine

### Example 15: 3-(2,3-Dihydro-1H-pyrrolo[3,2-b]pyridin-1-yl)-N,N-dimethyl-3-(thiophen-2-yl)propan-1-amine

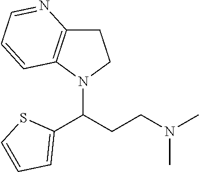 [SMILES: CN(C)CCC(c1cccs1)N1CCc2ncccc21]

### Step 1. Methyl 3-(2,3-dihydro-1H-pyrrolo[3,2-b]pyridin-1-yl)-3-(thiophen-2-yl)propanoate

Following the experimental procedure described for the preparation of Step 1 of Example 2 using suitable starting materials, the title compound was obtained.

### Step 2. 3-(2,3-Dihydro-1H-pyrrolo[3,2-b]pyridin-1-yl)-3-(thiophen-2-yl)propanoic Acid, Sodium Salt

A solution of the product obtained in Step 1 (287 mg, 0.95 mmol) in a mixture of THF (0.95 mL) and 1 M NaOH aqueous solution (0.95 mL, 0.95 mmol) was stirred at 50° C. overnight. The solvent was removed under vacuum to give the title compound as a crude product that was directly used in the following step (281 mg, quant. yield assumed).

### Step 3. 3-(2,3-Dihydro-1H-pyrrolo[3,2-b]pyridin-1-yl)-N,N-dimethyl-3-(thiophen-2-yl)propanamide

A mixture of the product obtained in Step 2 (281 mg, 0.95 mmol), HATU (434 mg, 1.14 mmol), DIPEA (0.75 mL, 4.3 mmol) and dimethylamine hydrochloride (388 mg, 4.7 mmol) in DMF (13 mL) was stirred at r.t. overnight. The reaction mixture was diluted with EtOAc and the organic phase was sequentially washed with NaHCO₃ sat. sol., water and brine, dried over MgSO₄, filtered and concentrated to dryness to give the title compound as a crude product that was directly used in the following step (128 mg, 44% yield).

### Step 4. Title Compound

Following the experimental procedure described for the preparation of Step 3 of Example 2 using the product obtained in Step 3 as starting material, the title compound was obtained (32 mg, 26% yield).

HPLC retention time (method D): 3.67 min; MS: 288.0 (M+H).

### Examples 16 and 17: (S)-3-(2,3-Dihydro-1H-pyrrolo[3,2-b]pyridin-1-yl)-N-methyl-3-(thiophen-2-yl)propan-1-amine and (R)-3-(2,3-dihydro-1H-pyrrolo[3,2-b]pyridin-1-yl)-N-methyl-3-(thiophen-2-yl)propan-1-amine

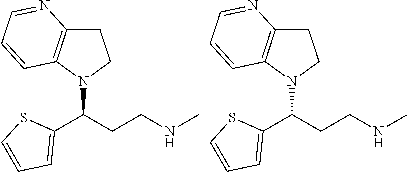

Starting from Example 3, a chiral preparative HPLC separation (column: Chiralpak IC; temperature: ambient; flow: 12 mL/min; eluent: n-Heptane/(IPA+0.3% DEA) 85/15 v/v) was carried out to give the title compounds.

### Examples 18 and 19: (R)-3-(2,3-Dihydro-1H-pyrrolo[3,2-b]pyridin-1-yl)-3-(5-fluorothiophen-2-yl)-N-methylpropan-1-amine and (S)-3-(2,3-dihydro-1H-pyrrolo[3,2-b]pyridin-1-yl)-3-(5-fluorothiophen-2-yl)-N-methylpropan-1-amine

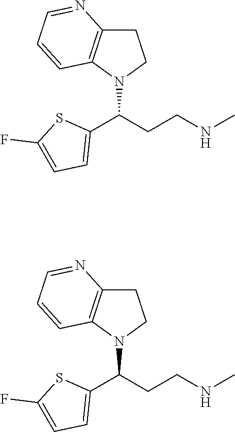 [SMILES: CNCC[C@H](c1ccc(F)s1)N1CCc2ncccc21 ; CNCC[C@@H](c1ccc(F)s1)N1CCc2ncccc21]

Starting from Example 6, a chiral preparative HPLC separation (column: Chiralcel OJ; temperature: ambient; flow: 10 mL/min; eluent: n-Heptane/(EtOH+0.2% DEA) 96:4 v/v) was carried out to give the title compounds.

### Examples 20 and 21: (R)-3-(3,3-Dimethyl-2,3-dihydro-1H-pyrrolo[3,2-b]pyridin-1-yl)-N-methyl-3-(thiophen-2-yl)propan-1-amine and (S)-3-(3,3-dimethyl-2,3-dihydro-1H-pyrrolo[3,2-b]pyridin-1-yl)-N-methyl-3-(thiophen-2-yl)propan-1-amine

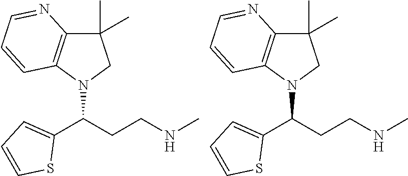

### Step 1. (S,E)-4-Benzyl-3-(3-(thiophen-2-yl)acryloyl)oxazolidin-2-one

To a solution of (E)-3-(thiophen-2-yl)acrylic acid (1.0 g, 6.49 mmol) in dry THF (31 mL), cooled at −30° C. under nitrogen, TEA (2.7 mL, 19.5 mmol) and pivaloyl chloride (0.88 mL, 0.86 mmol) were added dropwise and the mixture was stirred at −30° C. for 2 h. Then, lithium chloride (0.33 g, 7.78 mmol) and (S)-4-benzyl-2-oxazolidinone (1.26 g, 7.13 mmol) were added and the reaction mixture was stirred at r.t. overnight. Aqueous NH₄Cl sat. sol. and EtOAc were added, the phases were separated and the aqueous phase was extracted with EtOAc. The combined organic phases were washed with brine, dried over MgSO₄ and concentrated to dryness. The crude product was purified by flash chromatography, silica gel, gradient CH/EtOAc 100:0 to CH/EtOAc 0:100 to give the title compound (1.17 g, 58% yield).

### Step 2a and 2b. (S)-4-Benzyl-3-((R)-3-(3,3-dimethyl-2,3-dihydro-1H-pyrrolo[3,2-b]pyridin-1-yl)-3-(thiophen-2-yl)propanoyl)oxazolidin-2-one and (S)-4-benzyl-3-((S)-3-(3,3-dimethyl-2,3-dihydro-1H-pyrrolo[3,2-b]pyridin-1-yl)-3-(thiophen-2-yl)propanoyl)oxazolidin-2-one

To a solution of 3,3-dimethyl-2,3-dihydro-1H-pyrrolo[3,2-b]pyridine (224 mg, 1.51 mmol) in dry THF (11 mL), cooled at −78° C. under nitrogen, LDA solution (1.5 M in THF/ethylbenzene/heptane, 1.2 mL, 1.8 mmol) was added dropwise and the mixture was stirred at −78° C. for 30 min. Then, a solution of the product obtained in Step 1 (430 mg, 1.37 mmol) in dry THF (11 mL) was slowly added and the reaction mixture was stirred at −78° C. for 4 h. Aqueous NH₄Cl sat. sol. and EtOAc were added and the mixture was allowed to warm-up. The phases were separated and the aqueous phase was extracted with EtOAc. The combined organic phases were dried over MgSO₄ and concentrated to dryness. The crude product was purified by flash chromatography, silica gel, gradient CH/EtOAc 100:0 to CH/EtOAc 0:100 to give the title compounds Step 2a (117 mg, 18% yield) and Step 2b (209 mg 33% yield), together with a mixed fraction.

### Step 3a and 3b. (R)-3-(3,3-Dimethyl-2,3-dihydro-1H-pyrrolo[3,2-b]pyridin-1-yl)-N-methyl-3-(thiophen-2-yl)propanamide and (S)-3-(3,3-dimethyl-2,3-dihydro-1H-pyrrolo[3,2-b]pyridin-1-yl)-N-methyl-3-(thiophen-2-yl)propanamide

In a sealed tube, a mixture of the product obtained in Step 2a (117 mg, 0.25 mmol) and methylamine (33 wt % in EtOH, 1.58 mL, 12.7 mmol) was heated at 100° C. overnight. Then, the solvent was concentrated to dryness and the crude product was purified by flash chromatography, silica gel, gradient CH/EtOAc 100:0 to CH/EtOAc 0:100 to give the title compound (Step 3a, 67 mg, 84% yield).

Following an analogous procedure but starting from Step 2b, the title compound Step 3b was obtained.

### Step 4a and 4b. Title Compounds

To a solution of the product obtained in Step 3a (67 mg, 0.21 mmol) in THF (1.4 mL), borane-methyl sulfide complex (0.1 mL, 1.06 mmol) was added at r.t. and the reaction mixture was heated to reflux for 4 h under a nitrogen atmosphere. Then it was cooled to r.t. and it was concentrated to dryness. The residue was dissolved in MeOH (6 mL), 1 M HCl (4 mL) was added and the resulting mixture was heated to reflux for 1 h and then it was allowed to cool down to r.t. The mixture was basified with 1 M NaOH and it was extracted with EtOAc. The organic phases were combined, washed with brine, dried over MgSO₄, filtered and concentrated to dryness. The crude product was purified by flash chromatography, silica gel, gradient DCM to MeOH:DCM (1:4) to give Example 20 (23.7 mg, 37% yield).

Following an analogous procedure but starting from the product obtained in Step 3b, Example 21 was obtained.

HPLC retention time (method E): 3.68 min; MS: 302.1 (M+H).

This method was used for the preparation of Examples 22-61 using suitable starting materials:

| | | | | Ret | |
|---|---|---|---|---|---|
| | | | HPLC | time | MS |
| EX | Structure | Chemical name | Method | (min) | (M + H) |
| 22 | 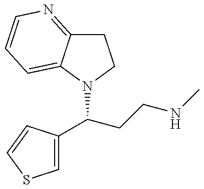 [SMILES: CNCC[C@H](c1ccsc1)N1CCc2ncccc21] | (R)-3-(2,3-Dihydro-1H- pyrrolo[3,2-b]pyridin-1- yl)-N-methyl-3- (thiophen-3-yl)propan-1- amine | E | 3.12 | 274.1 |
| 23 | 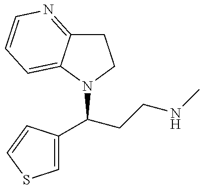 [SMILES: CNCC[C@@H](c1ccsc1)N1CCc2ncccc21] | (S)-3-(2,3-Dihydro-1H- pyrrolo[3,2-b]pyridin-1- yl)-N-methyl-3- (thiophen-3-yl)propan-1- amine | E | 3.19 | 274.1 |
| 24 | 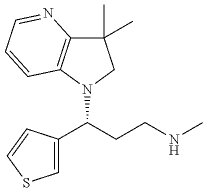 [SMILES: CNCC[C@H](c1ccsc1)N1CC(C)(C)c2ncccc21] | (R)-3-(3,3-Dimethyl-2,3- dihydro-1H-pyrrolo[3,2- b]pyridin-1-yl)-N-methyl- 3-(thiophen-3-yl)propan- 1-amine | E | 3.84 | 302.1 |
| 25 | 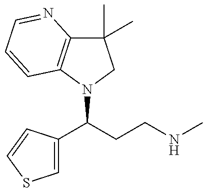 [SMILES: CNCC[C@@H](c1ccsc1)N1CC(C)(C)c2ncccc21] | (S)-3-(3,3-Dimethyl-2,3- dihydro-1H-pyrrolo[3,2- b]pyridin-1-yl)-N-methyl- 3-(thiophen-3-yl)propan- 1-amine | E | 3.82 | 302.1 |
| 26 | 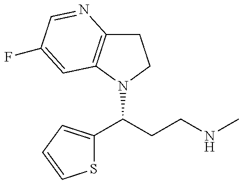 [SMILES: CNCC[C@H](c1cccs1)N1CCc2ncc(F)cc21] | (R)-3-(6-Fluoro-2,3- dihydro-1H-pyrrolo[3,2- b]pyridin-1-yl)-N-methyl- 3-(thiophen-2-yl)propan- 1-amine | E | 3.65 | 292.1 |
| 27 | 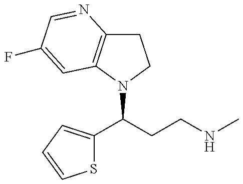 [SMILES: CNCC[C@@H](c1cccs1)N1CCc2ncc(F)cc21] | (S)-3-(6-Fluoro-2,3- dihydro-1H-pyrrolo[3,2- b]pyridin-1-yl)-N-methyl- 3-(thiophen-2-yl)propan- 1-amine | E | 3.70 | 292.1 |
| 28 | 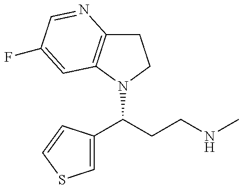 [SMILES: CNCC[C@H](c1ccsc1)N1CCc2ncc(F)cc21] | (R)-3-(6-Fluoro-2,3- dihydro-1H-pyrrolo[3,2- b]pyridin-1-yl)-N-methyl- 3-(thiophen-3-yl)propan- 1-amine | E | 3.70 | 292.1 |
| 29 | 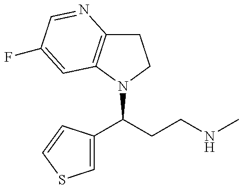 [SMILES: CNCC[C@@H](c1ccsc1)N1CCc2ncc(F)cc21] | (S)-3-(6-Fluoro-2,3- dihydro-1H-pyrrolo[3,2- b]pyridin-1-yl)-N-methyl- 3-(thiophen-3-yl)propan- 1-amine | E | 3.68 | 292.1 |
| 30 | 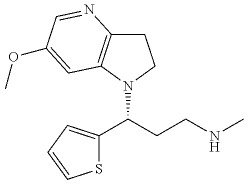 | (R)-3-(6-Methoxy-2,3- dihydro-1H-pyrrolo[3,2- b]pyridin-1-yl)-N-methyl- 3-(thiophen-2-yl)propan- 1-amine | E | 3.55 | 304.1 |
| 31 | 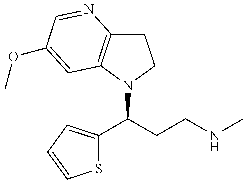 | (S)-3-(6-Methoxy-2,3- dihydro-1H-pyrrolo[3,2- b]pyridin-1-yl)-N-methyl- 3-(thiophen-2-yl)propan- 1-amine | E | 3.52 | 304.1 |
| 32 | 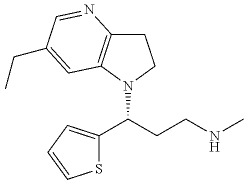 [SMILES: CCc1cnc2c(c1)N([C@H](CCNC)c1cccs1)CC2] | (R)-3-(6-Ethyl-2,3- dihydro-1H-pyrrolo[3,2- b]pyridin-1-yl)-N-methyl- 3-(thiophen-2-yl)propan- 1-amine | E | 4.01 | 302.1 |
| 33 | 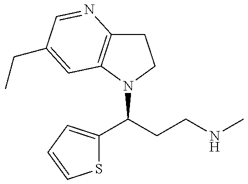 [SMILES: CCc1cnc2c(c1)N([C@@H](CCNC)c1cccs1)CC2] | (S)-3-(6-Ethyl-2,3- dihydro-1H-pyrrolo[3,2- b]pyridin-1-yl)-N-methyl- 3-(thiophen-2-yl)propan- 1-amine | E | 3.97 | 302.1 |
| 34 | 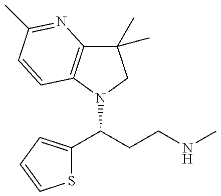 | (R)-N-methyl-3- (thiophen-2-yl)-3-(3,3,5- trimethyl-2,3-dihydro- 1H-pyrrolo[3,2-b]pyridin- 1-yl)propan-1-amine | E | 4.09 | 316.1 |
| 35 | 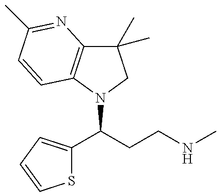 | (S)-N-methyl-3- (thiophen-2-yl)-3-(3,3,5- trimethyl-2,3-dihydro- 1H-pyrrolo[3,2-b]pyridin- 1-yl)propan-1-amine | E | 4.13 | 316.1 |
| 36 | 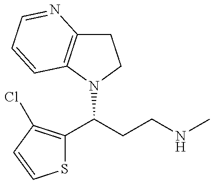 [SMILES: CNCC[C@H](c1sccc1Cl)N1CCc2ncccc21] | (R)-3-(3-Chlorothiophen- 2-yl)-3-(2,3-dihydro-1H- pyrrolo[3,2-b]pyridin-1- yl)-N-methylpropan-1- amine | E | 3.25 | 308.0 |
| 37 | 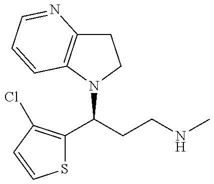 [SMILES: CNCC[C@@H](c1sccc1Cl)N1CCc2ncccc21] | (S)-3-(3-Chlorothiophen- 2-yl)-3-(2,3-dihydro-1H- pyrrolo[3,2-b]pyridin-1- yl)-N-methylpropan-1- amine | E | 3.27 | 308.1 |
| 38 | 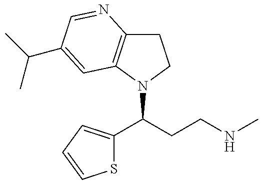 [SMILES: CNCC[C@@H](c1cccs1)N1CCc2ncc(C(C)C)cc21] | (S)-3-(6-Isopropyl-2,3- dihydro-1H-pyrrolo[3,2- b]pyridin-1-yl)-N-methyl- 3-(thiophen-2-yl)propan- 1-amine | E | 3.80 | 316.1 |
| 39 | 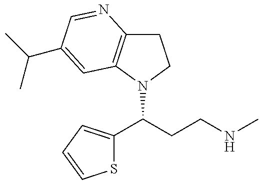 [SMILES: CNCC[C@H](c1cccs1)N1CCc2ncc(C(C)C)cc21] | (R)-3-(6-Isopropyl-2,3- dihydro-1H-pyrrolo[3,2- b]pyridin-1-yl)-N-methyl- 3-(thiophen-2-yl)propan- 1-amine | E | 3.93 | 316.1 |
| 40 | 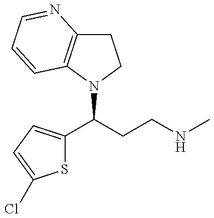 [SMILES: CNCC[C@@H](c1ccc(Cl)s1)N1CCc2ncccc21] | (S)-3-(5-Chlorothiophen- 2-yl)-3-(2,3-dihydro-1H- pyrrolo[3,2-b]pyridin-1- yl)-N-methylpropan-1- amine | E | 3.62 | 308.0 |
| 41 | 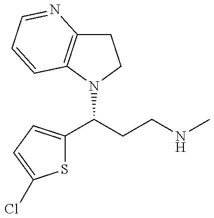 [SMILES: CNCC[C@H](c1ccc(Cl)s1)N1CCc2ncccc21] | (R)-3-(5-Chlorothiophen- 2-yl)-3-(2,3-dihydro-1H- pyrrolo[3,2-b]pyridin-1- yl)-N-methylpropan-1- amine | E | 3.62 | 308.0 |
| 42 | 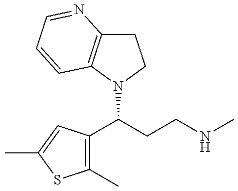 | (R)-3-(2,3-Dihydro-1H- pyrrolo[3,2-b]pyridin-1- yl)-3-(2,5- dimethylthiophen-3-yl)- N-methylpropan-1- amine | E | 3.83 | 302.1 |
| 43 | 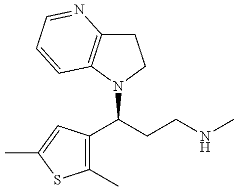 | (S)-3-(2,3-Dihydro-1H- pyrrolo[3,2-b]pyridin-1- yl)-3-(2,5- dimethylthiophen-3-yl)- N-methylpropan-1- amine | E | 3.85 | 302.1 |
| 44 | 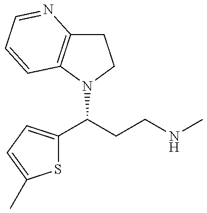 [SMILES: CNCC[C@H](c1ccc(C)s1)N1CCc2ncccc21] | (R)-3-(2,3-Dihydro-1H- pyrrolo[3,2-b]pyridin-1- yl)-N-methyl-3-(5- methylthiophen-2- yl)propan-1-amine | E | 3.46 | 288.1 |
| 45 | 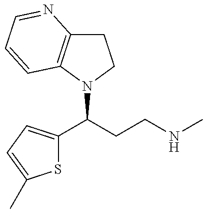 [SMILES: CNCC[C@@H](c1ccc(C)s1)N1CCc2ncccc21] | (S)-3-(2,3-Dihydro-1H- pyrrolo[3,2-b]pyridin-1- yl)-N-methyl-3-(5- methylthiophen-2- yl)propan-1-amine | E | 3.45 | 288.1 |
| 46 | 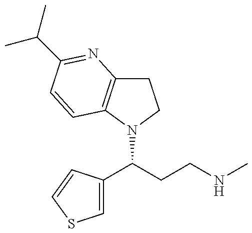 [SMILES: CNCC[C@H](c1ccsc1)N1CCc2nc(C(C)C)ccc21] | (R)-3-(5-Isopropyl-2,3- dihydro-1H-pyrrolo[3,2- b]pyridin-1-yl)-N-methyl- 3-(thiophen-3-yl)propan- 1-amine | E | 4.48 | 315.9 |
| 47 | 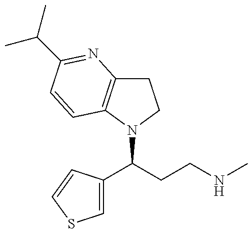 [SMILES: CNCC[C@@H](c1ccsc1)N1CCc2nc(C(C)C)ccc21] | (S)-3-(5-Isopropyl-2,3- dihydro-1H-pyrrolo[3,2- b]pyridin-1-yl)-N-methyl- 3-(thiophen-3-yl)propan- 1-amine | E | 4.48 | 315.9 |
| 48 | 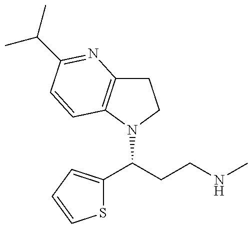 [SMILES: CNCC[C@H](c1cccs1)N1CCc2nc(C(C)C)ccc21] | (R)-3-(5-Isopropyl-2,3- dihydro-1H-pyrrolo[3,2- b]pyridin-1-yl)-N-methyl- 3-(thiophen-2-yl)propan- 1-amine | E | 4.52 | 315.9 |
| 49 | 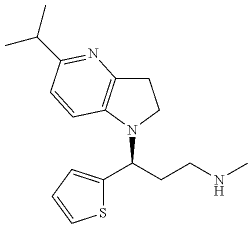 [SMILES: CNCC[C@@H](c1cccs1)N1CCc2nc(C(C)C)ccc21] | (S)-3-(5-Isopropyl-2,3- dihydro-1H-pyrrolo[3,2- b]pyridin-1-yl)-N-methyl- 3-(thiophen-2-yl)propan- 1-amine | E | 4.57 | 315.9 |
| 50 | 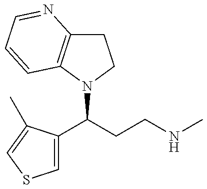 [SMILES: CNCC[C@@H](c1cscc1C)N1CCc2ncccc21] | (S)-3-(2,3-Dihydro-1H- pyrrolo[3,2-b]pyridin-1- yl)-N-methyl-3-(4- methylthiophen-3- yl)propan-1-amine | E | 3.39 | 287.9 |
| 51 | 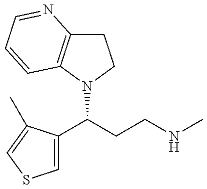 [SMILES: CNCC[C@H](c1cscc1C)N1CCc2ncccc21] | (R)-3-(2,3-Dihydro-1H- pyrrolo[3,2-b]pyridin-1- yl)-N-methyl-3-(4- methylthiophen-3- yl)propan-1-amine | E | 3.42 | 287.9 |
| 52 | 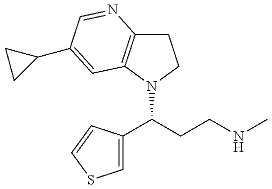 [SMILES: CNCC[C@H](c1ccsc1)N1CCc2ncc(C3CC3)cc21] | (R)-3-(6-Cyclopropyl- 2,3-dihydro-1H- pyrrolo[3,2-b]pyridin-1- yl)-N-methyl-3- (thiophen-3-yl)propan-1- amine | E | 3.80 | 313.9 |
| 53 | 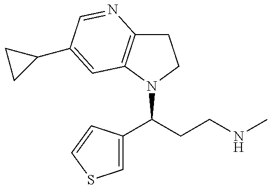 [SMILES: CNCC[C@@H](c1ccsc1)N1CCc2ncc(C3CC3)cc21] | (S)-3-(6-Cyclopropyl- 2,3-dihydro-1H- pyrrolo[3,2-b]pyridin-1- yl)-N-methyl-3- (thiophen-3-yl)propan-1- amine | E | 3.80 | 313.9 |
| 54 | 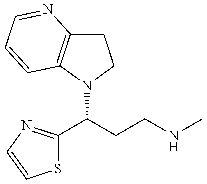 [SMILES: CNCC[C@H](c1nccs1)N1CCc2ncccc21] | (R)-3-(2,3-Dihydro-1H- pyrrolo[3,2-b]pyridin-1- yl)-N-methyl-3-(thiazol- 2-yl)propan-1-amine | E | 2.59 | 274.9 |
| 55 | 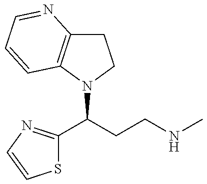 [SMILES: CNCC[C@@H](c1nccs1)N1CCc2ncccc21] | (S)-3-(2,3-Dihydro-1H- pyrrolo[3,2-b]pyridin-1- yl)-N-methyl-3-(thiazol- 2-yl)propan-1-amine | E | 2.61 | 274.9 |
| 56 | 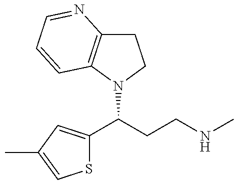 | (R)-3-(2,3-Dihydro-1H- pyrrolo[3,2-b]pyridin-1- yl)-N-methyl-3-(4- methylthiophen-2- yl)propan-1-amine | E | 3.39 | 287.9 |
| 57 | 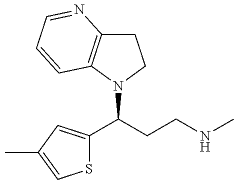 | (S)-3-(2,3-Dihydro-1H- pyrrolo[3,2-b]pyridin-1- yl)-N-methyl-3-(4- methylthiophen-2- yl)propan-1-amine | E | 3.42 | 287.9 |
| 58 | 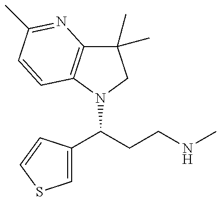 | (R)-N-methyl-3- (thiophen-3-yl)-3-(3,3,5- trimethyl-2,3-dihydro- 1H-pyrrolo[3,2-b]pyridin- 1-yl)propan-1-amine | E | 3.76 | 315.9 |
| 59 | 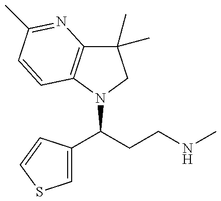 | (S)-N-methyl-3- (thiophen-3-yl)-3-(3,3,5- trimethyl-2,3-dihydro- 1H-pyrrolo[3,2-b]pyridin- 1-yl)propan-1-amine | E | 3.59 | 315.9 |
| 60 | 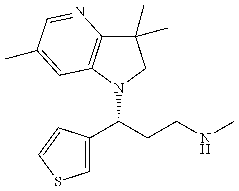 | (R)-N-methyl-3- (thiophen-3-yl)-3-(3,3,6- trimethyl-2,3-dihydro- 1H-pyrrolo[3,2-b]pyridin- 1-yl)propan-1-amine | E | 3.80 | 315.9 |
| 61 | 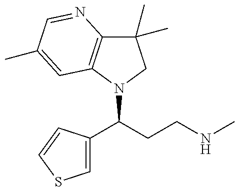 | (S)-N-methyl-3- (thiophen-3-yl)-3-(3,3,6- trimethyl-2,3-dihydro- 1H-pyrrolo[3,2-b]pyridin- 1-yl)propan-1-amine | E | 3.80 | 315.9 |

### Examples 62 and 63: (R)—N-methyl-3-(5-methyl-2,3-dihydro-1H-pyrrolo[3,2-b]pyridin-1-yl)-3-(thiophen-3-yl)propan-1-amine and (S)—N-methyl-3-(5-methyl-2,3-dihydro-1H-pyrrolo[3,2-b]pyridin-1-yl)-3-(thiophen-3-yl)propan-1-amine

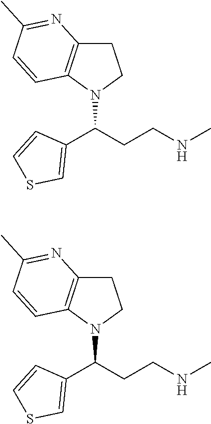

### Step 1. Ethyl 3-(5-methyl-2,3-dihydro-1H-pyrrolo[3,2-b]pyridin-1-yl)-3-(thiophen-3-yl)propanoate

Following the procedure described for the preparation of Step 1 of Example 2 but using (E)-ethyl 3-(thiophen-3-yl)acrylate and 5-methyl-2,3-dihydro-1H-pyrrolo[3,2-b]pyridine as starting materials, the title compound was obtained (243 mg, 42% yield).

### Step 2. 3-(5-Methyl-2,3-dihydro-1H-pyrrolo[3,2-b]pyridin-1-yl)-3-(thiophen-3-yl)propanoic Acid

Starting from the product obtained in Step 1 and following the experimental procedure described in Step 3 of Intermediate 5, the title compound was obtained (220 mg, quant. yield).

### Step 3a and 3b. (S)-4-Benzyl-3-((R)-3-(5-methyl-2,3-dihydro-1H-pyrrolo[3,2-b]pyridin-1-yl)-3-(thiophen-3-yl)propanoyl)oxazolidin-2-one and (S)-4-benzyl-3-((S)-3-(5-methyl-2,3-dihydro-1H-pyrrolo[3,2-b]pyridin-1-yl)-3-(thiophen-3-yl)propanoyl)oxazolidin-2-one

To a solution of the product obtained in Step 2 (266 mg, 0.92 mmol) in dry THF (4.4 mL), cooled at −30° C. under nitrogen, TEA (0.39 mL, 2.77 mmol) and pivaloyl chloride (0.13 mL, 1.02 mmol) were added dropwise and the mixture was stirred at −30° C. for 4 h. Then, lithium chloride (47 mg, 1.11 mmol) and (S)-4-benzyl-2-oxazolidinone (180 mg, 1.02 mmol) were added and the reaction mixture was stirred at r.t. overnight. Aqueous NH₄Cl sat. sol. and EtOAc were added, the phases were separated and the aqueous phase was extracted with EtOAc. The combined organic phases were washed with brine, dried over MgSO₄ and concentrated to dryness. The crude product was purified by flash chromatography, silica gel, gradient CH/EtOAc 100:0 to CH/EtOAc 0:100 to give the title compounds Step 3a (68 mg) and Step 3b (68 mg), together with 198 mg of a mixed fraction (74% global yield).

### Step 4a and 4b. (R)—N-methyl-3-(5-methyl-2,3-dihydro-1H-pyrrolo[3,2-b]pyridin-1-yl)-3-(thiophen-3-yl)propanamide and (S)—N-methyl-3-(5-methyl-2,3-dihydro-1H-pyrrolo[3,2-b]pyridin-1-yl)-3-(thiophen-3-yl)propanamide

Starting from the product obtained in Step 3a and following the experimental procedure described in Step 3a of Example 20, the title compound was obtained (Step 4a, 34 mg, 74% yield).

Following an analogous procedure but starting from Step 3b, the title compound Step 4b was obtained.

### Step 5a and 5b. Title Compounds

Starting from the product obtained in Step 4a and following the experimental procedure described in Step 4a of Example 20, the title compound was obtained (Step 5a, 18 mg, 56% yield).

Following an analogous procedure but starting from Step 4b, the title compound Step 5b was obtained.

HPLC retention time (method E): 3.39 min; MS: 287.9 (M+H).

Following the method described for the preparation of Example 2 but using suitable starting materials, Examples 64-75 were obtained:

| | | | | Ret | |
|---|---|---|---|---|---|
| | | | HPLC | time | Ms |
| EX | Structure | Chemical name | Method | (min) | (M + H) |
| 64 | 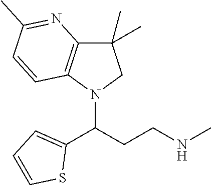 | N-methyl-3-(thiophen-2- yl)-3-(3,3,5-trimethyl- 2,3-dihydro-1H- pyrrolo[3,2-b]pyridin-1- yl)propan-1-amine | E | 3.88 | 315.9 |
| 65 | 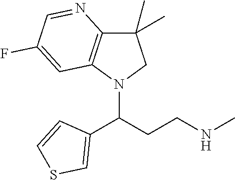 [SMILES: CNCCC(c1ccsc1)N1CC(C)(C)c2ncc(F)cc21] | 3-(6-Fluoro-3,3- dimethyl-2,3-dihydro- 1H-pyrrolo[3,2-b]pyridin- 1-yl)-N-methyl-3- (thiophen-3-yl)propan-1- amine | E | 4.23 | 320.1 |
| 66 | 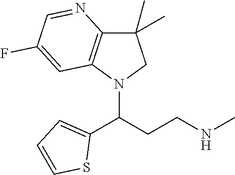 [SMILES: CNCCC(c1cccs1)N1CC(C)(C)c2ncc(F)cc21] | 3-(6-Fluoro-3,3- dimethyl-2,3-dihydro- 1H-pyrrolo[3,2-b]pyridin- 1-yl)-N-methyl-3- (thiophen-2-yl)propan-1- amine | E | 4.08 | 320.1 |
| 67 | 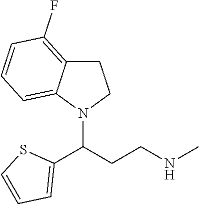 [SMILES: CNCCC(c1cccs1)N1CCc2c(F)cccc21] | 3-(4-Fluoroindolin-1-yl)- N-methyl-3-(thiophen-2- yl)propan-1-amine | E | 4.46 | 291.1 |
| 68 | 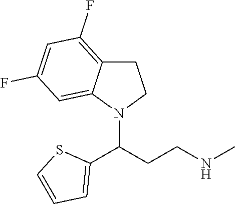 [SMILES: CNCCC(c1cccs1)N1CCc2c(F)cc(F)cc21] | 3-(4,6-Difluoroindolin-1- yl)-N-methyl-3- (thiophen-2-yl)propan-1- amine | E | 4.69 | 309.1 |
| 69 | 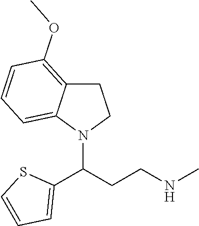 [SMILES: CNCCC(c1cccs1)N1CCc2c(OC)cccc21] | 3-(4-Methoxyindolin-1- yl)-N-methyl-3- (thiophen-2-yl)propan-1- amine | E | 4.52 | 303.1 |
| 70 | 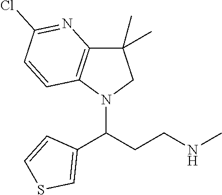 [SMILES: CNCCC(c1ccsc1)N1CC(C)(C)c2nc(Cl)ccc21] | 3-(5-Chloro-3,3- dimethyl-2,3-dihydro- 1H-pyrrolo[3,2-b]pyridin- 1-yl)-N-methyl-3- (thiophen-3-yl)propan-1- amine | E | 4.45 | 336.1 |
| 71 | 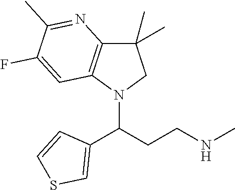 [SMILES: CNCCC(c1ccsc1)N1CC(C)(C)c2nc(C)c(F)cc21] | 3-(6-Fluoro-3,3,5- trimethyl-2,3-dihydro- 1H-pyrrolo[3,2-b]pyridin- 1-yl)-N-methyl-3- (thiophen-3-yl)propan-1- amine | E | 3.97 | 334.1 |
| 72 | 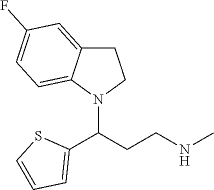 [SMILES: CNCCC(c1cccs1)N1CCc2cc(F)ccc21] | 3-(5-Fluoroindolin-1-yl)- N-methyl-3-(thiophen-2- yl)propan-1-amine | E | 4.30 | 291.1 |
| 73 | 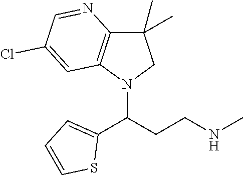 [SMILES: CNCCC(c1cccs1)N1CC(C)(C)c2ncc(Cl)cc21] | 3-(6-Chloro-3,3- dimethyl-2,3-dihydro- 1H-pyrrolo[3,2-b]pyridin- 1-yl)-N-methyl-3- (thiophen-2-yl)propan-1- amine⁽¹⁾ | E | 4.46 | 336.1 |
| 74 | 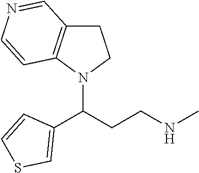 [SMILES: CNCCC(c1ccsc1)N1CCc2cnccc21] | 3-(2,3-Dihydro-1H- pyrrolo[3,2-c]pyridin-1- yl)-N-methyl-3- (thiophen-3-yl)propan-1- amine⁽¹⁾ | F | 0.29 | 274.1 |
| 75 | 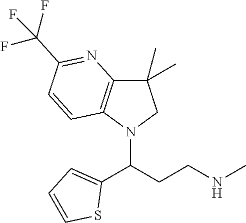 [SMILES: CNCCC(c1cccs1)N1CC(C)(C)c2nc(C(F)(F)F)ccc21] | 3-(3,3-Dimethyl-5- (trif1uoromethyl)-2,3- dihydro-1H-pyrrolo[3,2- b]pyridin-1-yl)-N-methyl- 3-(thiophen-2-yl)propan- 1-amine⁽¹⁾ | E | 5.21 | 370.1 |

⁽¹⁾The corresponding 4-methoxyphenyl ester was used in Step 1 instead of the alkyl ester.

Following the method described for the preparation of Examples 20 and 21 but using suitable starting materials, Examples 76-91 were obtained:

| | | | | Ret | |
|---|---|---|---|---|---|
| | | | HPLC | time | MS |
| EX | Structure | Chemical name | Method | (min) | (M + H) |
| 76 | 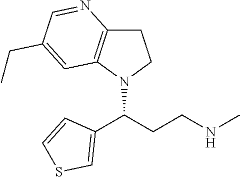 [SMILES: CCc1cnc2c(c1)N([C@H](CCNC)c1ccsc1)CC2] | (R)-3-(6-Ethyl-2,3- dihydro-1H-pyrrolo[3,2- b]pyridin-1-yl)-N-methyl- 3-(thiophen-3-yl)propan- 1-amine | E | 3.60 | 301.9 |
| 77 | 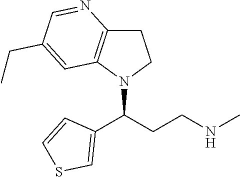 [SMILES: CCc1cnc2c(c1)N([C@@H](CCNC)c1ccsc1)CC2] | (S)-3-(6-Ethyl-2,3- dihydro-1H-pyrrolo[3,2- b]pyridin-1-yl)-N-methyl- 3-(thiophen-3-yl)propan- 1-amine | E | 3.69 | 301.9 |
| 78 | 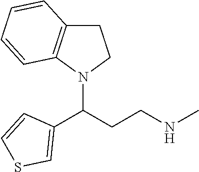 [SMILES: CNCCC(c1ccsc1)N1CCc2ccccc21] | 3-(Indolin-1-yl)-N- methyl-3-(thiophen-3- yl)propan-1-amine⁽¹⁾ | E | 4.26 | 272.9 |
| 79 | 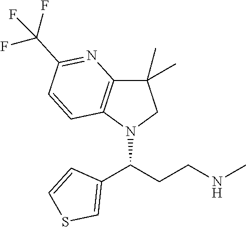 [SMILES: CNCC[C@H](c1ccsc1)N1CC(C)(C)c2nc(C(F)(F)F)ccc21] | 3-(3,3-Dimethyl-5- (trifluoromethyl)-2,3- dihydro-1H-pyrrolo[3,2- b]pyridin-1-yl)-N-methyl- 3-(thiophen-3-yl)propan- 1-amine⁽¹⁾ | E | 4.94 | 369.9 |
| 80 | 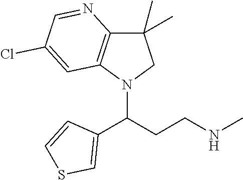 [SMILES: CNCCC(c1ccsc1)N1CC(C)(C)c2ncc(Cl)cc21] | 3-(6-Chloro-3,3- dimethyl-2,3-dihydro- 1H-pyrrolo[3,2-b]pyridin- 1-yl)-N-methyl-3- (thiophen-3-yl)propan-1- amine⁽¹⁾ | E | 4.39 | 335.9 |
| 81 | 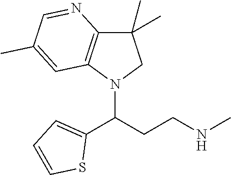 | N-methyl-3-(thiophen-2- yl)-3-(3,3,6-trimethyl- 2,3-dihydro-1H- pyrrolo[3,2-b]pyridin-1- yl)propan-1-amine⁽¹⁾ | E | 3.87 | 315.9 |
| 82 | 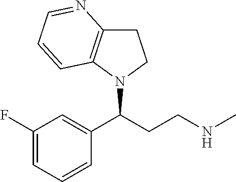 [SMILES: CNCC[C@@H](c1cccc(F)c1)N1CCc2ncccc21] | (S)-3-(2,3-Dihydro-1H- pyrrolo[3,2-b]pyridin-1- yl)-3-(3-fluorophenyl)-N- methylpropan-1-amine | E | 3.48 | 286.1 |
| 83 | 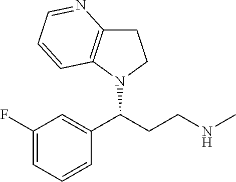 [SMILES: CNCC[C@H](c1cccc(F)c1)N1CCc2ncccc21] | (R)-3-(2,3-Dihydro-1H- pyrrolo[3,2-b]pyridin-1- yl)-3-(3-fluorophenyl)-N- methylpropan-1-amine | E | 3.48 | 286.1 |
| 84 | 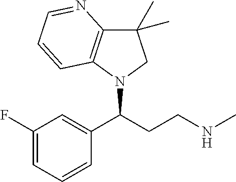 [SMILES: CNCC[C@@H](c1cccc(F)c1)N1CC(C)(C)c2ncccc21] | (S)-3-(3,3-Dimethyl-2,3- dihydro-1H-pyrrolo[3,2- b]pyridin-1-yl)-3-(3- fluorophenyl)-N- methylpropan-1-amine | E | 4.27 | 314.1 |
| 85 | 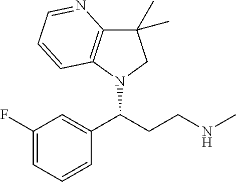 [SMILES: CNCC[C@H](c1cccc(F)c1)N1CC(C)(C)c2ncccc21] | (R)-3-(3,3-Dimethyl-2,3- dihydro-1H-pyrrolo[3,2- b]pyridin-1-yl)-3-(3- fluorophenyl)-N- methylpropan-1-amine | E | 4.33 | 314.1 |
| 86 | 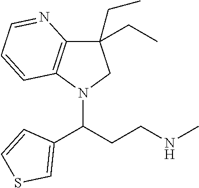 [SMILES: CCC1(CC)CN(C(CCNC)c2ccsc2)c2cccnc21] | 3-(3,3-Diethyl-2,3- dihydro-1H-pyrrolo[3,2- b]pyridin-1-yl)-N-methyl- 3-(thiophen-3-yl)propan- 1-amine⁽¹⁾ | E | 4.88 | 330.1 |
| 87 | 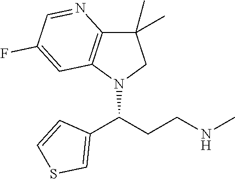 [SMILES: CNCC[C@H](c1ccsc1)N1CC(C)(C)c2ncc(F)cc21] | (R)-3-(6-Fluoro-3,3- dimethyl-2,3-dihydro- 1H-pyrrolo[3,2-b]pyridin- 1-yl)-N-methyl-3- (thiophen-3-yl)propan-1- amine | E | 3.71 | 320.2 |
| 88 | 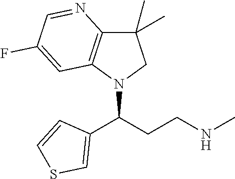 [SMILES: CNCC[C@@H](c1ccsc1)N1CC(C)(C)c2ncc(F)cc21] | (S)-3-(6-Fluoro-3,3- dimethyl-2,3-dihydro- 1H-pyrrolo[3,2-b]pyridin- 1-yl)-N-methyl-3- (thiophen-3-yl)propan-1- amine | E | 3.72 | 320.2 |
| 89 | 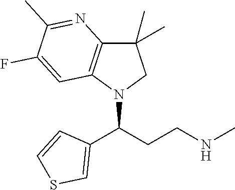 [SMILES: CNCC[C@@H](c1ccsc1)N1CC(C)(C)c2nc(C)c(F)cc21] | (S)-3-(6-Fluoro-3,3,5- trimethyl-2,3-dihydro- 1H-pyrrolo[3,2-b]pyridin- 1-yl)-N-methyl-3- (thiophen-3-yl)propan-1- amine | E | 4.01 | 334.1 |
| 90 | 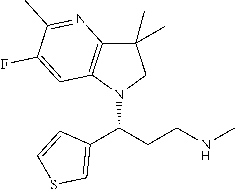 [SMILES: CNCC[C@H](c1ccsc1)N1CC(C)(C)c2nc(C)c(F)cc21] | (R)-3-(6-Fluoro-3,3,5- trimethyl-2,3-dihydro- 1H-pyrrolo[3,2-b]pyridin- 1-yl)-N-methyl-3- (thiophen-3-yl)propan-1- amine | E | 4.01 | 334.1 |
| 91 | 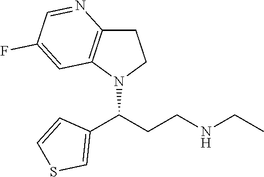 [SMILES: CCNCC[C@H](c1ccsc1)N1CCc2ncc(F)cc21] | (R)-N-ethyl-3-(6-fluoro- 2,3-dihydro-1H- pyrrolo[3,2-b]pyridin-1- yl)-3-(thiophen-3- yl)propan-1-amine⁽²⁾ | E | 3.43 | 306.1 |

⁽¹⁾Diastereomers were not separated in Step 2, rendering racemic compounds in Step 3 and Step 4.
⁽²⁾Ethylamine solution was used in Step 3 instead of methylamine

### Example 92: 3,3-Dimethyl-1-(3-(methylamino)-1-(thiophen-2-yl)propyl)indoline-6-carbonitrile

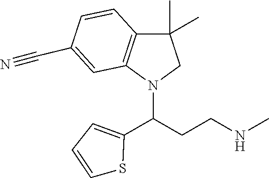 [SMILES: CNCCC(c1cccs1)N1CC(C)(C)c2ccc(C#N)cc21]

### Step 1. 1-(3-Chloro-1-(thiophen-2-yl)propyl)-3,3-dimethylindoline-6-carbonitrile

To a solution of 3-chloro-1-(thiophen-2-yl)propan-1-ol (176 mg, 0.98 mmol) in THF (7.7 mL), cooled at 0° C., TEA (0.42 mL, 3 mmol) and methanesulfonyl chloride (0.09 mL, 1.19 mmol) were added dropwise and the mixture was stirred at 0° C. for 1 h. A solution of 3,3-dimethylindoline-6-carbonitrile (206 mg, 1.19 mmol) in THF (1 mL) was added and the mixture was stirred at r.t. for 3 days and finally it was heated to reflux for an additional day to get the reaction to completion. It was then allowed to cool, and it was diluted with aqueous NaHCO₃ sat. sol and EtOAc. The phases were separated and the aqueous phase was extracted with EtOAc. The combined organic phases were dried over MgSO₄ and concentrated to dryness. The crude product was purified by flash chromatography, silica gel, gradient CH/EtOAc 100:0 to 50:50 to give the title compound (267 mg, 81% yield).

### Step 2. Title Compound

In a sealed tube, a solution of the product obtained in Step 1 (267 mg, 0.81 mmol) and methylamine (33 wt % in EtOH, 5 mL, 40 mmol) was heated at 100° C. overnight. Then, the solvent was concentrated. The crude product was purified by flash chromatography, silica gel, gradient DCM to MeOH:DCM (1:4) to give the title compound (96 mg, 36% yield).

HPLC retention time (method E): 4.54 min; MS: 326.1 (M+H).

This method was used for the preparation of Examples 93-101 using suitable starting materials:

| | | | | Ret | |
|---|---|---|---|---|---|
| | | | HPLC | time | MS |
| EX | Structure | Chemical name | Method | (min) | (M + H) |
| 93 | 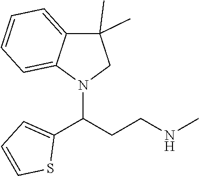 [SMILES: CNCCC(c1cccs1)N1CC(C)(C)c2ccccc21] | 3-(3,3-Dimethylindolin-1- yl)-N-methyl-3- (thiophen-2-yl)propan-1- amine | E | 5.18 | 301.1 |
| 94 | 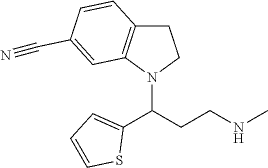 [SMILES: CNCCC(c1cccs1)N1CCc2ccc(C#N)cc21] | 1-(3-(Methylamino)-1- (thiophen-2- yl)propyl)indoline-6- carbonitrile | E | 4.43 | 298.1 |
| 95 | 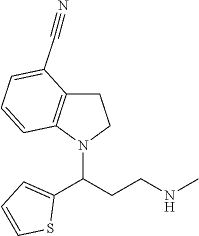 [SMILES: CNCCC(c1cccs1)N1CCc2c(C#N)cccc21] | 1-(3-(Methylamino)-1- (thiophen-2- yl)propyl)indoline-4- carbonitrile | E | 4.35 | 298.1 |
| 96 | 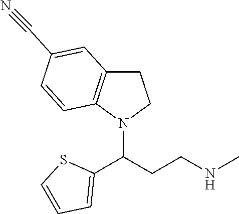 [SMILES: CNCCC(c1cccs1)N1CCc2cc(C#N)ccc21] | 1-(3-(Methylamino)-1- (thiophen-2- yl)propyl)indoline-5- carbonitrile | E | 4.07 | 298.1 |
| 97 | 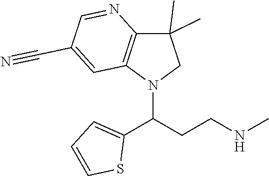 [SMILES: CNCCC(c1cccs1)N1CC(C)(C)c2ncc(C#N)cc21] | 3,3-Dimethyl-1-(3- (methylamino)-1- (thiophen-2-yl)propyl)- 2,3-dihydro-1H- pyrrolo[3,2-b]pyridine-6- carbonitrile | E | 3.91 | 327.1 |
| 98 | 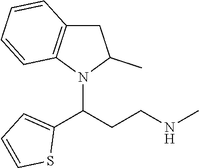 [SMILES: CNCCC(c1cccs1)N1c2ccccc2CC1C] | N-methyl-3-(2- methylindolin-1-yl)-3- (thiophen-2-yl)propan-1- amine | E | 4.45 | 287.1 |
| 99 | 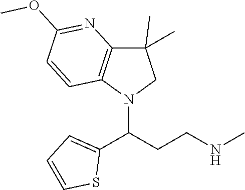 | 3-(5-Methoxy-3,3- dimethyl-2,3-dihydro- 1H-pyrrolo[3,2-b]pyridin- 1-yl)-N-methyl-3- (thiophen-2-yl)propan-1- amine | E | 4.26 | 332.1 |
| 100 | 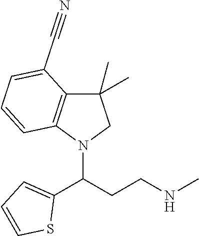 [SMILES: CNCCC(c1cccs1)N1CC(C)(C)c2c(C#N)cccc21] | 3,3-Dimethyl-1-(3- (methylamino)-1- (thiophen-2- yl)propyl)indoline-4- carbonitrile | E | 4.51 | 326.1 |
| 101 | 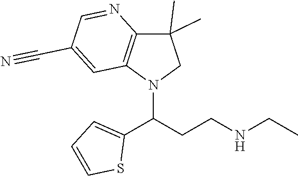 [SMILES: CCNCCC(c1cccs1)N1CC(C)(C)c2ncc(C#N)cc21] | 1-(3-(Ethylamino)-1- (thiophen-2-yl)propyl)- 3,3-dimethyl-2,3- dihydro-1H-pyrrolo[3,2- b]pyridine-6-carbonitrile | E | 4.17 | 341.1 |

### Examples 102 and 103: (S)—N-methyl-3-(6-methyl-2,3-dihydro-1H-pyrrolo[3,2-b]pyridin-1-yl)-3-(thiophen-3-yl)propan-1-amine and (R)—N-methyl-3-(6-methyl-2,3-dihydro-1H-pyrrolo[3,2-b]pyridin-1-yl)-3-(thiophen-3-yl)propan-1-amine

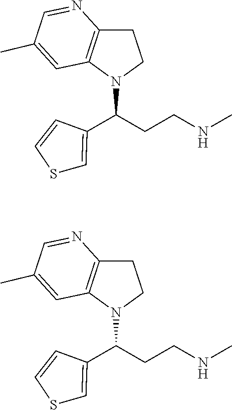

### Step 1. Ethyl 3-(6-methyl-2,3-dihydro-1H-pyrrolo[3,2-b]pyridin-1-yl)-3-(thiophen-3-yl)propanoate

Following the experimental procedure described for the preparation of Step 1 of Example 2 using (E)-ethyl 3-(thiophen-3-yl)acrylate and 6-methyl-2,3-dihydro-1H-pyrrolo[3,2-b]pyridine as starting materials, the title compound was obtained.

### Step 2. 3-(6-Methyl-2,3-dihydro-1H-pyrrolo[3,2-b]pyridin-1-yl)-3-(thiophen-3-yl)propanoic Acid

To a solution of the product obtained in Step 1 (346 mg, 1.09 mmol) in THF (4.5 mL), 1 N NaOH aqueous solution (5.5 mL, 5.5 mmol) was added and the mixture was stirred at r.t. overnight. Then, pH was adjusted to 4-5 with 1 N HCl. The precipitated solids were collected by filtration, washed with water and cold Et₂O and finally dried under vacuum to give the title compound (319 mg, quant. yield)

### Step 3a and 3b. (S)-4-Benzyl-3-((S)-3-(6-methyl-2,3-dihydro-1H-pyrrolo[3,2-b]pyridin-1-yl)-3-(thiophen-3-yl)propanoyl)oxazolidin-2-one and (S)-4-benzyl-3-((R)-3-(6-methyl-2,3-dihydro-1H-pyrrolo[3,2-b]pyridin-1-yl)-3-(thiophen-3-yl)propanoyl)oxazolidin-2-one

Following the experimental procedure described for the preparation of Step 1 of Examples 20 and 21 using the compound obtained in Step 2 as starting material, the title compounds were obtained.

### Step 4a and 4b. (S)—N-Methyl-3-(6-methyl-2,3-dihydro-1H-pyrrolo[3,2-b]pyridin-1-yl)-3-(thiophen-3-yl)propanamide and (R)—N-methyl-3-(6-methyl-2,3-dihydro-1H-pyrrolo[3,2-b]pyridin-1-yl)-3-(thiophen-3-yl)propanamide

Following the experimental procedure described for the preparation of Step 3a and 3b of Examples 20 and 21, using the compounds obtained in Step 3a and 3b as starting materials, the title compounds were obtained.

### Step 5a and 5b. Title Compounds

Following the experimental procedure described for the preparation of Step 4a and 4b of Examples 20 and 21, using the compounds obtained in Step 4a and 4b as starting materials, the title compounds were obtained.

HPLC retention time (method E): 3.44 min; MS: 287.9 (M+H).

### Example 104: 3,3-Dimethyl-1-(3-(methylamino)-1-(thiophen-3-yl)propyl)-2,3-dihydro-1H-pyrrolo[3,2-b]pyridine-5-carbonitrile Hydrochloride

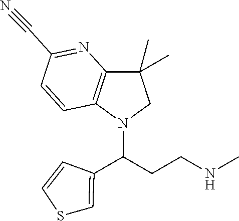 [SMILES: CNCCC(c1ccsc1)N1CC(C)(C)c2nc(C#N)ccc21]

### Step 1. Tert-Butyl (3-(5-chloro-3,3-dimethyl-2,3-dihydro-1H-pyrrolo[3,2-b]pyridin-1-yl)-3-(thiophen-3-yl)propyl)(methyl)carbamate

A solution of Example 70 (173 mg, 0.52 mmol) in DCM (8 mL) was cooled at 0° C. Then, TEA (0.1 mL, 0.77 mmol) and a solution of di-tert-butyl dicarbonate (124 mg, 0.57 mmol) in DCM (8 mL) were sequentially added and the mixture was stirred at r.t. overnight. Water was added, the layers were separated and the aqueous phase was back extracted with DCM. The combined organic phases were washed with brine, dried over MgSO₄ and concentrated under vacuum to give the title compound as a crude product that was used as such (267 mg, overweight, quant. yield assumed).

### Step 2. Tert-Butyl (3-(5-cyano-3,3-dimethyl-2,3-dihydro-1H-pyrrolo[3,2-b]pyridin-1-yl)-3-(thiophen-3-yl)propyl)(methyl)carbamate

A mixture of the product obtained in Step 1 (130 mg, 0.3 mmol), SPhos (12 mg, 0.03 mmol), tris(dibenzylideneacetone)dipalladium(0) (14 mg, 0.02 mmol) and zinc cyanide (53 mg, 0.45 mmol) in DMF (1.6 mL) was placed in a microwave vial. The system was inertized with argon and it was irradiated under microwave heating at 150° C. for 70 min. After cooling, aqueous NH₄Cl sat. sol. and EtOAc were added, the phases were separated and the aqueous phase was extracted with EtOAc. The combined organic phases were dried over MgSO₄ and concentrated to dryness. The residue was purified by flash chromatography, silica gel, gradient DCM to MeOH:DCM (1:4) to give the title compound (34 mg, 27% yield).

### Step 3. Title Compound

HCl (0.4 mL, 1 M solution in Et₂O, 0.4 mmol) was carefully added to a solution of the product obtained in Step 2 (34 mg, 0.08 mmol) in MeOH (1 mL) and the mixture was stirred at r.t. overnight. It was then concentrated to dryness and the residue was dried under vacuum to yield the title compound (29 mg, quant. yield).

HPLC retention time (method E): 3.85 min; MS: 327.1 (M+H).

### Examples 105 and 106: (S)-3-(6-Fluoro-3,3-dimethyl-2,3-dihydro-1H-pyrrolo[3,2-b]pyridin-1-yl)-N-methyl-3-(thiophen-2-yl)propan-1-amine and (R)-3-(6-fluoro-3,3-dimethyl-2,3-dihydro-1H-pyrrolo[3,2-b]pyridin-1-yl)-N-methyl-3-(thiophen-2-yl)propan-1-amine

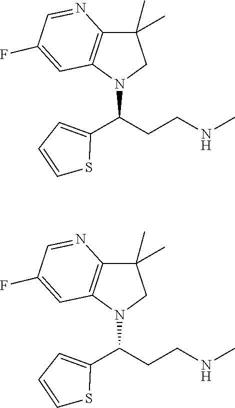 [SMILES: CNCC[C@@H](c1cccs1)N1CC(C)(C)c2ncc(F)cc21 ; CNCC[C@H](c1cccs1)N1CC(C)(C)c2ncc(F)cc21]

Starting from Example 66, a chiral preparative HPLC separation (column: Chiralcel ODH; temperature: ambient; flow: 2.5 mL/min; eluent: n-Heptane/(EtOH+0.2% DEA) 90:10 v/v) was carried out to give the title compounds.

### Examples 107 and 108: (S)-3-(3,3-Dimethyl-5-(trifluoromethyl)-2,3-dihydro-1H-pyrrolo[3,2-b]pyridin-1-yl)-N-methyl-3-(thiophen-3-yl)propan-1-amine and (R)-3-(3,3-dimethyl-5-(trifluoromethyl)-2,3-dihydro-1H-pyrrolo[3,2-b]pyridin-1-yl)-N-methyl-3-(thiophen-3-yl)propan-1-amine

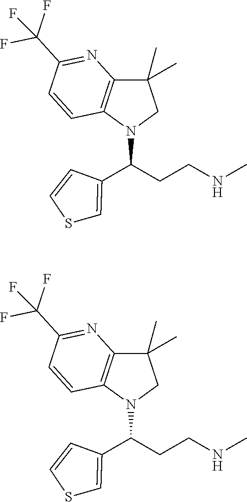 [SMILES: CNCC[C@@H](c1ccsc1)N1CC(C)(C)c2nc(C(F)(F)F)ccc21 ; CNCC[C@H](c1ccsc1)N1CC(C)(C)c2nc(C(F)(F)F)ccc21]

Starting from Example 79, a chiral preparative HPLC separation (column: Chiralcel ODH; temperature: ambient; flow: 2.5 mL/min; eluent: n-Heptane/(EtOH+0.2% DEA) 70:30 v/v) was carried out to give the title compounds.

### Examples 109 and 110: (S)-3-(6-Chloro-3,3-dimethyl-2,3-dihydro-1H-pyrrolo[3,2-b]pyridin-1-yl)-N-methyl-3-(thiophen-2-yl)propan-1-amine and (R)-3-(6-chloro-3,3-dimethyl-2,3-dihydro-1H-pyrrolo[3,2-b]pyridin-1-yl)-N-methyl-3-(thiophen-2-yl)propan-1-amine

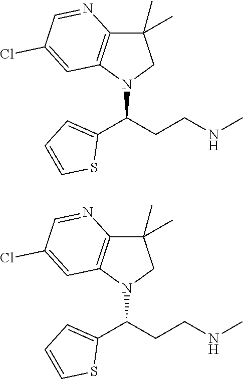

Starting from Example 73, a chiral preparative HPLC separation (column: Chiralcel ODH; temperature: ambient; flow: 0.5 mL/min; eluent: n-Heptane/(EtOH+0.2% DEA) 95:5 v/v) was carried out to give the title compounds.

### Examples 111 and 112: (S)-1-(3-(Methylamino)-1-(thiophen-2-yl)propyl)indoline-4-carbonitrile and (S)-1-(3-(methylamino)-1-(thiophen-2-yl)propyl)indoline-4-carbonitrile

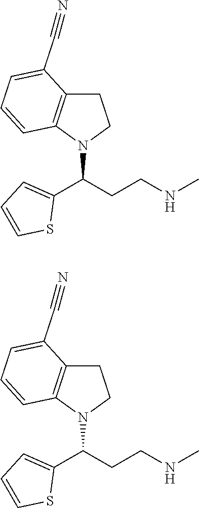 [SMILES: CNCC[C@@H](c1cccs1)N1CCc2c(C#N)cccc21 ; CNCC[C@H](c1cccs1)N1CCc2c(C#N)cccc21]

Starting from Example 95, a chiral preparative HPLC separation (column: Chiralcel ODH; temperature: ambient; flow: 10 mL/min; eluent: n-Heptane/(EtOH+0.2% DEA) 95:5 v/v) was carried out to give the title compounds.

### Examples 113 and 114: (S)-3-(5-Methoxy-3,3-dimethyl-2,3-dihydro-1H-pyrrolo[3,2-b]pyridin-1-yl)-N-methyl-3-(thiophen-2-yl)propan-1-amine and (R)-3-(5-methoxy-3,3-dimethyl-2,3-dihydro-1H-pyrrolo[3,2-b]pyridin-1-yl)-N-methyl-3-(thiophen-2-yl)propan-1-amine

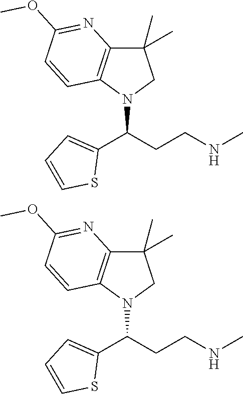

Starting from Example 99, a chiral preparative HPLC separation (column: Chiralcel ODH; temperature: ambient; flow: 10 mL/min; eluent: n-Heptane/(EtOH+0.2% DEA) 98:2 v/v) was carried out to give the title compounds.

### Examples 115 and 116: (S)-3,3-Dimethyl-1-(3-(methylamino)-1-(thiophen-2-yl)propyl)-2,3-dihydro-1H-pyrrolo[3,2-b]pyridine-6-carbonitrile and (R)-3,3-dimethyl-1-(3-(methylamino)-1-(thiophen-2-yl)propyl)-2,3-dihydro-1H-pyrrolo[3,2-b]pyridine-6-carbonitrile

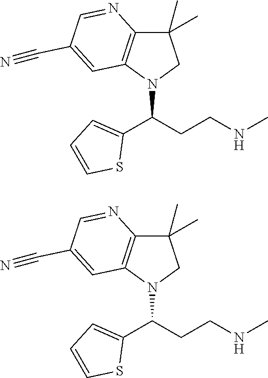

Starting from Example 97, a chiral preparative HPLC separation (column: Chiralcel ODH; temperature: ambient; flow: 10 mL/min; eluent: n-Heptane/(EtOH+0.3% DEA) 95:5 v/v) was carried out to give the title compounds.

### Examples 117, 118 and 119: (S)—N-methyl-3-((R)-2-methylindolin-1-yl)-3-(thiophen-2-yl)propan-1-amine, (R)—N-methyl-3-((S)-2-methylindolin-1-yl)-3-(thiophen-2-yl)propan-1-amine and (S/R)—N-methyl-3-((S/R)-2-methylindolin-1-yl)-3-(thiophen-2-yl)propan-1-amine

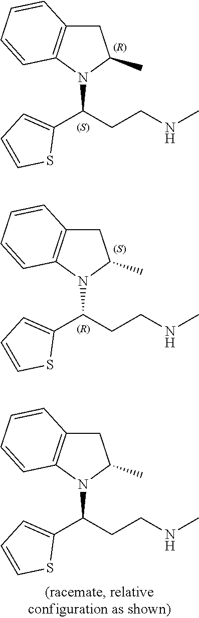

Starting from Example 98, a chiral preparative HPLC separation (column: Chiralcel ODH; temperature: ambient; flow: 10 mL/min; eluent: n-Heptane/(EtOH+0.2% DEA) 98:2 v/v) was carried out to give Examples 117 and 118 as pure enantiomers and Example 118 as a racemate with relative configuration as shown.

### Examples 120 and 121: (S)-1-(3-(Ethylamino)-1-(thiophen-2-yl)propyl)-3,3-dimethyl-2,3-dihydro-1H-pyrrolo[3,2-b]pyridine-6-carbonitrile and (R)-1-(3-(ethylamino)-1-(thiophen-2-yl)propyl)-3,3-dimethyl-2,3-dihydro-1H-pyrrolo[3,2-b]pyridine-6-carbonitrile

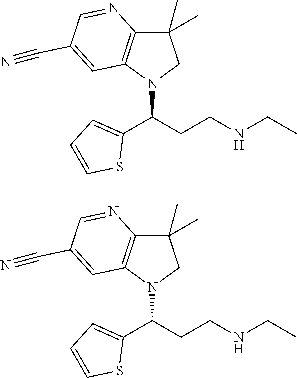

Starting from Example 101, a chiral preparative HPLC separation (column: Chiralpak IC; temperature: ambient; flow: 10 mL/min; eluent: n-Heptane/(EtOH+0.3% DEA) 95:5 v/v) was carried out to give the title compounds.

Examples of Biological Activity

Binding Assay to Human α2δ-1 Subunit of Cav2.2 Calcium Channel.

Human α2δ-1 enriched membranes (2.5 μg) were incubated with 15 nM of radiolabeled [3H]-Gabapentin in assay buffer containing Hepes-KOH 10 mM, pH 7.4.

NSB (non specific binding) was measured by adding 10 μM pregabalin. After 60 min incubation at 27° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, pH 7.4.

Filter plates were dried at 60° C. for 1 hour and 30 μl of scintillation cocktail were added to each well before radioactivity reading.

Readings were performed in a Trilux 1450 Microbeta radioactive counter (Perkin Elmer).

Binding Assay to Human Norepinephrine Transporter (NET).

Human norepinephrine transporter (NET) enriched membranes (5 μg) were incubated with 5 nM of radiolabeled [3H]-Nisoxetin in assay buffer containing 50 mM Tris-HCl, 120 mM NaCl, 5 mM KCl, pH 7.4.

NSB (non specific binding) was measured by adding 1 μM. After 60 min incubation at 4° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, 0.9% NaCl, pH 7.4. Filter plates were dried at 60° C. for 1 hour and 30 μl of scintillation cocktail were added to each well before radioactivity reading.

Readings were performed in a Trilux 1450 Microbeta radioactive counter (Perkin Elmer).

The following scale has been adopted for representing the binding to the α2δ-1 receptor expressed as Ki:
+ Ki-α2δ-1>=3000 nM
++ 500 nM<Ki-α2δ-1<3000 nM
+++ 100 nM<Ki-α2δ-1<500 nM
++++ Ki-α2δ-1<100 nM

For the NET receptor, the following scale has been adopted for representing the binding expressed as Ki:
+ Ki-NET>=1000 nM
++ 500 nM<Ki-NET<1000 nM
+++ 100 nM<Ki-NET<500 nM
++++ Ki-NET<100 nM

The results of the binding for the α2δ-1 and the NET receptor are shown in Table 1:

TABLE 1

| | Ki (nM) | Ki (nM) |
|---|---|---|
| Example | NET | alpha2delta |
| nr | Hum | Hum |
| 1 | ++++ | ++ |
| 2 | + | ++ |
| 3 | ++ | ++ |
| 4 | +++ | + |
| 5 | + | +++ |
| 6 | ++ | ++ |
| 7 | ++ | + |
| 8 | + | +++ |
| 9 | + | + |
| 10 | +++ | +++ |
| 11 | ++ | +++ |
| 12 | ++ | +++ |
| 13 | ++ | ++ |
| 14 | ++ | +++ |
| 15 | + | + |
| 16 | +++ | +++ |
| 17 | +++ | ++ |
| 18 | ++ | ++ |
| 19 | ++ | +++ |
| 20 | + | +++ |
| 21 | +++ | ++ |
| 22 | + | ++ |
| 23 | +++ | +++ |
| 24 | + | +++ |
| 25 | +++ | ++ |
| 26 | + | +++ |
| 27 | + | +++ |
| 28 | ++ | +++ |
| 29 | ++ | +++ |
| 30 | + | +++ |
| 31 | ++ | + |
| 32 | +++ | +++ |
| 33 | ++ | + |
| 34 | + | ++++ |
| 35 | ++++ | +++ |
| 36 | +++ | + |
| 37 | +++ | + |
| 38 | + | + |
| 39 | + | +++ |
| 40 | ++ | +++ |
| 41 | + | ++ |
| 42 | + | + |
| 43 | + | + |
| 44 | + | ++ |
| 45 | + | ++ |
| 46 | + | ++ |
| 47 | + | ++ |
| 48 | + | ++ |
| 49 | + | + |
| 50 | +++ | + |
| 51 | + | + |
| 52 | ++ | ++++ |
| 53 | + | ++ |
| 54 | + | + |
| 55 | + | + |
| 56 | + | ++ |
| 57 | + | ++ |
| 58 | + | +++ |
| 59 | +++ | +++ |
| 60 | + | +++ |
| 61 | + | ++ |
| 62 | + | ++ |
| 63 | ++ | ++ |
| 64 | +++ | ++++ |
| 65 | + | ++++ |
| 66 | + | +++ |
| 67 | ++++ | ++ |
| 68 | ++++ | ++ |
| 69 | ++++ | + |
| 71 | +++ | ++++ |
| 72 | +++ | + |
| 73 | + | +++ |
| 74 | + | ++ |
| 75 | + | +++ |
| 76 | +++ | +++ |
| 77 | +++ | ++ |
| 78 | ++++ | + |
| 79 | + | +++ |
| 80 | + | +++ |
| 81 | + | ++++ |
| 82 | ++ | ++ |
| 83 | +++ | + |
| 84 | +++ | + |
| 85 | + | + |
| 86 | + | ++ |
| 87 | + | +++ |
| 88 | ++ | +++ |
| 89 | +++ | +++ |
| 90 | + | ++++ |
| 91 | + | ++ |
| 92 | + | +++ |
| 93 | ++++ | ++ |
| 94 | + | +++ |
| 95 | ++++ | +++ |
| 96 | ++ | ++ |
| 97 | + | ++++ |
| 99 | +++ | +++ |
| 100 | + | ++++ |
| 101 | | ++++ |
| 102 | + | ++ |
| 103 | + | +++ |
| 104 | + | +++ |
| 105 | + | ++ |
| 106 | + | ++++ |
| 107 | +++ | +++ |
| 108 | + | +++ |
| 109 | + | + |
| 110 | + | +++ |
| 111 | ++++ | ++ |
| 112 | + | +++ |
| 113 | ++ | ++ |
| 114 | + | +++ |
| 115 | | ++++ |
| 116 | | ++ |
| 117 | ++++ | + |
| 118 | + | + |
| 119 | + | + |

1-16. (canceled)

17. A compound of general formula (I): 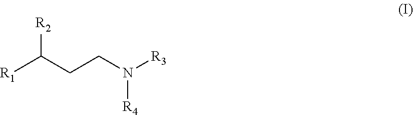
wherein:
R₁ is selected from an optionally substituted 5 or 6-membered aryl group or an optionally substituted 5 to 10-membered heteroaryl group having at least one heteroatom selected from the group consisting of N, O and S;
R₂ is 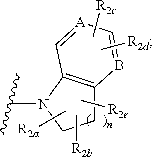
n is 1 or 2;
A and B independently represent a carbon atom leading to either —CH—, —CR_{2c}— or —CR_{2d}—; or a nitrogen atom with the proviso that, if one of A and B is nitrogen the other is a carbon atom, and with the proviso that when A and B are both carbon atoms, R₁ may not be phenyl;
R₂ₐ and R_{2b} are, independently from one another, a hydrogen atom or a branched or unbranched C₁₋₆ alkyl radical; or
R₂ₐ and R_{2b}, when attached to the same carbon atom, together with the carbon atom to which they are attached, may optionally form a spirocyclic structure;
R_{2c} and R_{2d} are, independently from one another, a hydrogen atom; a —(CH₂)ₘ—CN group, wherein m is 0 or 1; a halogen; a branched or unbranched C₁₋₆ alkyl radical; a C₁₋₆ alkylamino radical; an amino group; an hydroxy group; a C₁₋₆ alkoxy radical; C₁₋₆ haloalkoxy radical; an alkoxyalkyl C₁₋₆ radical; a C₃₋₆ cycloalkyl radical; a 5 or 6-membered heterocycloalkyl; a heterocycloalkylalkyl C₁₋₆; a C₁₋₆ haloalkyl radical; a —CF₃ group; an optionally substituted aryl group; an arylalkyl radical C; an optionally substituted 5 to 10-membered heteroaryl group having at least one heteroatom selected from the group consisting of N, O and S; or a heteroarylalkyl radical C₁₋₆;
R₂ₑ is a hydrogen atom: a ═O group; or a branched or unbranched C₁₋₆ alkyl radical;
R₃ and R₄ are, independently from one another, a hydrogen atom or a branched or unbranched optionally substituted C₁₋₆ alkyl radical;
or a pharmaceutically acceptable salt, co-crystal, isomer, prodrug or solvate thereof.

18. The compound according to claim 17, wherein R₁ represents a thiophene, a thiazole or a phenyl, which groups are optionally substituted by at least one substituent selected from the group consisting of halogen, branched or unbranched C₁₋₆-alkyl, C₁₋₆-alkoxy, C₁₋₆-haloalkoxy, C₁₋₆-haloalkyl, trihaloalkyl, CN and a hydroxyl group.

19. The compound according to claim 17, wherein R₁ represents a group selected from the group consisting of: 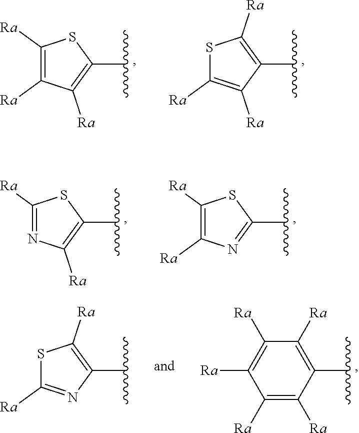
wherein each Rₐ independently represents a hydrogen atom, a halogen, branched or unbranched C₁₋₆ alkyl, C₁₋₆-alkoxy, C₁₋₆-haloalkoxy, C₁₋₆-haloalkyl, trihaloalkyl, CN or a hydroxyl group.

20. The compound according to claim 17, wherein R₂ represents a group selected from the group consisting of: 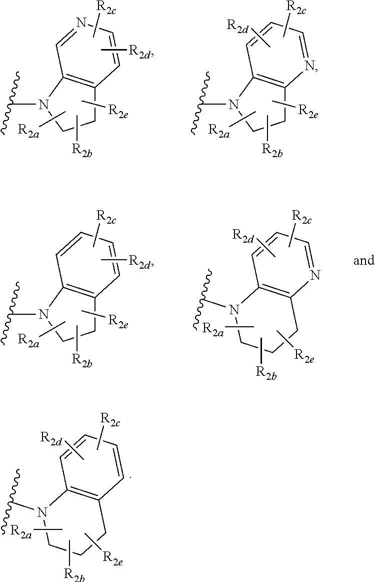
wherein R₂ₐ, R_{2b}, R_{2c}, R_{2d} and R₂ₑ are as defined in claim 17.

21. The compound according to claim 17, wherein R₂ₐ and R_{2b} independently represent hydrogen, methyl or ethyl.

22. The compound according to claim 17, wherein R₂ₐ and R_{2b}, are attached to the same carbon atom and both represent a methyl group or, together with the carbon atom to which they are attached, form a spirocyclopropyl.

23. The compound according to claim 17, wherein R₂ₑ represents a hydrogen atom, a ═O group, a methyl or an ethyl group.

24. The compound according to claim 17, wherein R_{2c} and R_{2d} are, independently from one another, a hydrogen atom; a —(CH₂)ₘ—CN group, wherein m is 0 or 1; a C₁₋₆ alkylamino radical; an amino group; an hydroxy group; a halogen; a branched or unbranched C₁₋₆ alkyl radical; a C₁₋₆ alkoxy radical; C₁₋₆ haloalkoxy radical, an alkoxyalkyl C₁₋₆ radical; or a C₃₋₆ cycloalkyl radical; a C₁₋₆ haloalkyl radical; —CF₃ group; an optionally substituted 5 or 6-membered aryl group; an arylalkyl radical C₁₋₆; or an optionally substituted 5 to 10-membered heteroaryl group having at least one heteroatom selected from the group consisting of N, O and S.

25. The compound according to claim 17, wherein R_{2c} and R_{2d} independently represent hydrogen, methyl, ethyl, isopropyl, halogen, methoxy, cyclopropyl, —CH₂—CN, —CN, —CH₂—N(CH₃)₂, methoxymethyl or a —CF₃ group.

26. The compound according to claim 17, wherein R₃ and R₄ independently represent hydrogen or a C₁₋₆ alkyl radical.

27. The compound according to claim 26, wherein R₃ and R₄ independently represent hydrogen, methyl or ethyl.

28. The compound according to claim 17, which is selected from the group consisting of:
3-(Indolin-1-yl)-N-methyl-3-(thiophen-2-yl)propan-1-amine;
3-(2,3-Dihydro-1H-pyrrolo[2,3-c]pyridin-1-yl)-N-methyl-3-(thiophen-2-yl)propan-1-amine;
3-(2,3-Dihydro-1H-pyrrolo[3,2-b]pyridin-1-yl)-N-methyl-3-(thiophen-2-yl)propan-1-amine;
3-(3,4-Dihydroquinolin-1(2H)-yl)-N-methyl-3-(thiophen-2-yl)propan-1-amine;
3-(3,4-Dihydro-1,5-naphthyridin-1(2H)-yl)-N-methyl-3-(thiophen-2-yl)propan-1-amine;
3-(2,3-Dihydro-1H-pyrrolo[3,2-b]pyridin-1-yl)-3-(5-fluorothiophen-2-yl)-N-methylpropan-1-amine;
3-(2,3-Dihydro-1H-pyrrolo[3,2-b]pyridin-1-yl)-N-methyl-3-phenylpropan-1-amine;
3-(2,3-Dihydro-1H-pyrrolo[3,2-b]pyridin-1-yl)-N-ethyl-3-(thiophen-2-yl)propan-1-amine;
3-(2,3-Dihydro-1H-pyrrolo[3,2-b]pyridin-1-yl)-3-(thiophen-2-yl)propan-1-amine;
3-(2,3-Dihydro-1H-pyrrolo[3,2-b]pyridin-1-yl)-N-methyl-3-(thiophen-3-yl)propan-1-amine;
N-methyl-3-(5-methyl-2,3-dihydro-1H-pyrrolo[3,2-b]pyridin-1-yl)-3-(thiophen-2-yl)propan-1-amine;
3-(3,3-Dimethyl-2,3-dihydro-1H-pyrrolo[3,2-b]pyridin-1-yl)-N-methyl-3-(thiophen-2-yl)propan-1-amine;
3-(3,3-Dimethyl-2,3-dihydro-1H-pyrrolo[3,2-b]pyridin-1-yl)-N-methyl-3-(thiophen-3-yl)propan-1-amine;
N-methyl-3-(6-methyl-2,3-dihydro-1H-pyrrolo[3,2-b]pyridin-1-yl)-3-(thiophen-3-yl)propan-1-amine;
3-(2,3-Dihydro-1H-pyrrolo[3,2-b]pyridin-1-yl)-N,N-dimethyl-3-(thiophen-2-yl)propan-1-amine;
(S)-3-(2,3-Dihydro-1H-pyrrolo[3,2-b]pyridin-1-yl)-N-methyl-3-(thiophen-2-yl)propan-1-amine;
(R)-3-(2,3-Dihydro-1H-pyrrolo[3,2-b]pyridin-1-yl)-N-methyl-3-(thiophen-2-yl)propan-1-amine;
(R)-3-(2,3-Dihydro-1H-pyrrolo[3,2-b]pyridin-1-yl)-3-(5-fluorothiophen-2-yl)-N-methylpropan-1-amine;
(S)-3-(2,3-Dihydro-1H-pyrrolo[3,2-b]pyridin-1-yl)-3-(5-fluorothiophen-2-yl)-N-methylpropan-1-amine;
(R)-3-(3,3-Dimethyl-2,3-dihydro-1H-pyrrolo[3,2-b]pyridin-1-yl)-N-methy-3-(thiophen-2-yl)propan-1-amine;
(S)-3-(3,3-Dimethyl-2,3-dihydro-H-pyrrolo[3,2-b]pyridin-1-yl)-N-methyl-3-(thiophen-2-yl)propan-1-amine;
(R)-3-(2,3-Dihydro-1H-pyrrolo[3,2-b]pyridin-1-yl)-N-methyl-3-(thiophen-3-yl)propan-1-amine;
(S)-3-(2,3-Dihydro-1H-pyrrolo[3,2-b]pyridin-1-yl)-N-methyl-3-(thiophen-3-yl)propan-1-amine;
(R)-3-(3,3-Dimethyl-2,3-dihydro-1H-pyrrolo[3,2-b]pyridin-1-yl)-N-methyl-3-(thiophen-3-yl)propan-1-amine;
(S)-3-(3,3-Dimethyl-2,3-dihydro-1H-pyrrolo[3,2-b]pyridin-1-yl)-N-methyl-3-(thiophen-3-yl)propan-1-amine;
(R)-3-(6-Fluoro-2,3-dihydro-1H-pyrrol[3,2-b]pyridin-1-yl)-N-methyl-3-(thiophen-2-yl)propan-1-amine;
(S)-3-(6-Fluoro-2,3-dihydro-1H-pyrrolo[3,2-b]pyridin-1-yl)-N-methyl-3-(thiophen-2-yl)propan-1-amine;
(R)-3-(6-Fluoro-2,3-dihydro-1H-pyrrolo[3,2-b]pyridin-1-yl)-N-methyl-3-(thiophen-3-yl)propan-1-amine;
(S)-3-(6-Fluoro-2,3-dihydro-1H-pyrrolo[3,2-b]pyridin-1-yl)-N-methy-3-(thiophen-3-yl)propan-1-amine;
(R)-3-(6-Methoxy-2,3-dihydro-1H-pyrrolo[3,2-b]pyridin-1-yl)-N-methyl-3-(thiophen-2-yl)propan-1-amine;
(S)-3-(6-Methoxy-2,3-dihydro-1H-pyrrolo[3,2-b]pyridin-1-yl)-N-methyl-3-(thiophen-2-yl)propan-1-amine;
(R)-3-(6-Ethyl-2,3-dihydro-1H-pyrrolo[3,2-b]pyridin-1-yl)-N-methyl-3-(thiophen-2-yl)propan-1-amine;
(S)-3-(6-Ethyl-2,3-dihydro-1H-pyrrolo[3,2-b]pyridin-1-yl)-N-methyl-3-(thiophen-2-yl)propan-1-amine;
(R)—N-methyl-3-(thiophen-2-yl)-3-(3,3,5-trimethyl-2,3-dihydro-1H-pyrrolo[3,2-b]pyridin-1-yl)propan-1-amine;
(S)—N-methyl-3-(thiophen-2-yl)-3-(3,3,5-trimethyl-2,3-dihydro-H-pyrrolo[3,2-b]pyridin-1-yl)propan-1-amine;
(R)-3-(3-Chlorothiophen-2-yl)-3-(2,3-dihydro-1H-pyrrolo[3,2-b]pyridin-1-yl)-N-methylpropan-1-amine;
(S)-3-(3-Chlorothiophen-2-yl)-3-(2,3-dihydro-H-pyrrolo[3,2-b]pyridin-1-yl)-N-methylpropan-1-amine;
(S)-3-(6-Isopropyl-2,3-dihydro-1H-pyrrolo[3,2-b]pyridin-1-yl)-N-methyl-3-(thiophen-2-yl)propan-1-amine;
(R)-3-(6-Isopropyl-2,3-dihydro-1H-pyrrolo[3,2-b]pyridin-1-yl)-N-methyl-3-(thiophen-2-yl)propan-1-amine;
(S)-3-(5-Chlorothiophen-2-yl)-3-(2,3-dihydro-1H-pyrrolo[3,2-b]pyridin-1-yl)-N-methylpropan-1-amine;
(R)-3-(5-Chlorothiophen-2-yl)-3-(2,3-dihydro-1H-pyrrolo[3,2-b]pyridin-1-yl)-N-methylpropan-1-amine;
(R)-3-(2,3-Dihydro-1H-pyrrolo[3,2-b]pyridin-1-yl)-3-(2,5-dimethylthiophen-3-yl)-N-methylpropan-1-amine;
(S)-3-(2,3-Dihydro-1H-pyrrolo[3,2-b]pyridin-1-yl)-3-(2,5-dimethylthiophen-3-yl)-N-methylpropan-1-amine;
(R)-3-(2,3-Dihydro-1H-pyrrolo[3,2-b]pyridin-1-yl)-N-methyl-3-(5-methylthiophen-2-yl)propan-1-amine;
(S)-3-(2,3-Dihydro-1H-pyrrolo[3,2-b]pyridin-1-yl)-N-methyl-3-(5-methylthiophen-2-yl)propan-1-amine;
(R)-3-(5-Isopropyl-2,3-dihydro-1H-pyrrolo[3,2-b]pyridin-1-yl)-N-methyl-3-(thiophen-3-yl)propan-1-amine;
(S)-3-(5-isopropyl-2,3-dihydro-1H-pyrrolo[32-b]pyridin-1-yl)-N-methyl-3-(thiophen-3-yl)propan-1-amine;
(R)-3-(5-Isopropyl-2,3-dihydro-1H-pyrrolo[3,2-b]pyridin-1-yl)-N-methyl-3-(thiophen-2-yl)propan-1-amine;
(S)-3-(5-Isopropyl-2,3-dihydro-1H-pyrrolo[3,2-b]pyridin-1-yl)-N-methyl-3-(thiophen-2-yl)propan-1-amine;
(S)-3-(2,3-Dihydro-1H-pyrrolo[3,2-b]pyridin-1-yl)-N-methy-3-(4-methylthiophen-3-yl)propan-1-amine;
(R)-3-(2,3-Dihydro-1H-pyrrolo[3,2-b]pyridin-1-yl)-N-methyl-3-(4-methylthiophen-3-yl)propan-1-amine;
(R)-3-(6-Cyclopropyl-2,3-dihydro-1H-pyrrolo[3,2-b]pyridin-1-yl)-N-methyl-3-(thiophen-3-yl)propan-1-amine;
(S)-3-(6-Cyclopropyl-2,3-dihydro-1H-pyrrolo[3,2-b]pyridin-1-yl)-N-methyl-3-(thiophen-3-yl)propan-1-amine;
(R)-3-(2,3-Dihydro-1H-pyrrolo[3,2-b]pyridin-1-yl)-N-methyl-3-(thiazol-2-yl)propan-1-amine;
(S)-3-(2,3-Dihydro-1H-pyrrolo[3,2-b]pyridin-1-yl)-N-methyl-3-(thiazol-2-yl)propan-1-amine;
(R)-3-(2,3-Dihydro-1H-pyrrolo[3,2-b]pyridin-1-yl)-N-methyl-3-(4-methylthiophen-2-yl)propan-1-amine;
(S)-3-(2,3-Dihydro-1H-pyrrol[3,2-b]pyridin-1-yl)-N-methyl-3-(4-methylthiophen-2-yl)propan-1-amine;
(R)—N-methyl-3-(thiophen-3-yl)-3-(3,3,5-trimethyl-2,3-dihydro-1H-pyrrolo[3,2-b]pyridin-1-yl)propan-1-amine;
(S)—N-methyl-3-(thiophen-3-yl)-3-(3,3,5-trimethyl-2,3-dihydro-1H-pyrrolo[3,2-b]pyridin-1-yl)propan-1-amine;
(R)—N-methyl-3-(thiophen-3-yl)-3-(3,3,6-trimethyl-2,3-dihydro-1H-pyrrolo[3,2-b]pyridin-1-yl)propan-1-amine;
(S)—N-methyl-3-(thiophen-3-yl)-3-(3,3,6-trimethyl-2,3-dihydro-1H-pyrrolo[3,2-b]pyridin-1-yl)propan-1-amine;
(R)—N-methyl-3-(5-methyl-2,3-dihydro-H-pyrrolo[3,2-b]pyridin-1-yl)-3-(thiophen-3-yl)propan-1-amine;
(S)—N-methyl-3-(5-methyl-2,3-dihydro-1H-pyrrolo[3,2-b]pyridin-1-yl)-3-(thiophen-3-yl)propan-1-amine;
N-methyl-3-(thiophen-2-yl)-3-(3,3,5-trimethyl-2,3-dihydro-1H-pyrrolo[3,2-b]pyridin-1-yl)propan-1-amine;
3-(6-Fluoro-3,3-dimethyl-2,3-dihydro-1H-pyrrolo[3,2-b]pyridin-1-yl)-N-methyl-3-(thiophen-3-yl)propan-1-amine;
3-(6-Fluoro-3,3-dimethyl-2,3-dihydro-1H-pyrrolo[3,2-b]pyridin-1-yl)-N-methyl-3-(thiophen-2-yl)propan-1-amine;
3-(4-Fluoroindolin-1-yl)-N-methyl-3-(thiophen-2-yl)propan-1-amine;
3-(4,6-Difluoroindolin-1-yl)-N-methyl-3-(thiophen-2-yl)propan-1-amine;
3-(4-Methoxyindolin-1-yl)-N-methyl-3-(thiophen-2-yl)propan-1-amine;
3-(5-Chloro-3,3-dimethyl-2,3-dihydro-1H-pyrrolo[3,2-b]pyridin-1-yl)-N-methyl-3-(thiophen-3-yl)propan-1-amine;
3-(6-Fluoro-3,3,5-trimethyl-2,3-dihydro-1H-pyrrolo[3,2-b]pyridin-1-yl)-N-methyl-3-(thiophen-3-yl)propan-1-amine;
3-(5-Fluoroindolin-1-yl)-N-methyl-3-(thiophen-2-yl)propan-1-amine;
3-(6-Chloro-3,3-dimethyl-2,3-dihydro-1H-pyrrolo[3,2-b]pyridin-1-yl)-N-methyl-3-(thiophen-2-yl)propan-1-amine;
3-(2,3-Dihydro-1H-pyrrolo[3,2-c]pyridin-1-yl)-N-methyl-3-(thiophen-3-yl)propan-1-amine;
3-(3,3-Dimethyl-5-(trifluoromethyl)-2,3-dihydro-1H-pyrrolo[3,2-b]pyridin-1-yl)-N-methyl-3-(thiophen-2-yl)propan-1-amine;
(R)-3-(6-Ethyl-2,3-dihydro-1H-pyrrolo[3,2-b]pyridin-1-yl)-N-methyl-3-(thiophen-3-yl)propan-1-amine;
(S)-3-(6-Ethyl-2,3-dihydro-1H-pyrrolo[3,2-b]pyridin-1-yl)-N-methyl-3-(thiophen-3-yl)propan-1-amine;
3-(Indolin-1-yl)-N-methyl-3-(thiophen-3-yl)propan-1-amine;
3-(3,3-Dimethyl-5-(trifluoromethyl)-2,3-dihydro-1H-pyrrolo[3,2-b]pyridin-1-yl)-N-methyl-3-(thiophen-3-yl)propan-1-amine;
3-(6-Chloro-3,3-dimethyl-2,3-dihydro-1H-pyrrolo[3,2-b]pyridin-1-yl)-N-methyl-3-(thiophen-3-yl)propan-1-amine;
N-methyl-3-(thiophen-2-yl)-3-(3,3,6-trimethyl-2,3-dihydro-1H-pyrrolo[3,2-b]pyridin-1-yl)propan-1-amine;
(S)-3-(2,3-Dihydro-1H-pyrrolo[3,2-b]pyridin-1-yl)-3-(3-(3-fluorophenyl)-N-methylpropan-1-amine;
(R)-3-(2,3-Dihydro-1H-pyrrolo[3,2-b]pyridin-1-yl)-3-(3-fluorophenyl)-N-methylpropan-1-amine;
(S)-3-(3,3-Dimethyl-2,3-dihydro-1H-pyrrolo[3,2-b]pyridin-1-yl)-3-fluorophenyl)-N-methylpropan-1-amine;
(R)-3-(3,3-Dimethyl-2,3-dihydro-1H-pyrrolo[3,2-b]pyridin-1-yl)-3-(3-fluorophenyl)-N-methylpropan-1-amine;
3-(3,3-Diethyl-2,3-dihydro-1H-pyrrolo[3,2-b]pyridin-1-yl)-N-methyl-3-(thiophen-3-yl)propan-1-amine;
(R)-3-(6-Fluoro-3,3-dimethyl-2,3-dihydro-1H-pyrrolo[3,2-b]pyridin-1-yl)-N-methyl-3-(thiophen-3-yl)propan-1-amine;
(S)-3-(6-Fluoro-3,3-dimethyl-2,3-dihydro-1H-pyrrolo[3,2-b]pyridin-1-yl)-N-methyl-3-(thiophen-3-yl)propan-1-amine;
(S)-3-(6-Fluoro-3,3,5-trimethyl-2,3-dihydro-1H-pyrrolo[3,2-b]pyridin-1-yl)-N-methyl-3-(thiophen-3-yl)propan-1-amine;
(R)-3-(6-Fluoro-3,3,5-trimethyl-2,3-dihydro-1H-pyrrolo[3,2-b]pyridin-1-yl)-N-methyl-3-(thiophen-3-yl)propan-1-amine;
(R)—N-ethyl-3-(6-fluoro-2,3-dihydro-1H-pyrrolo[3,2-b]pyridin-1-yl)-3-(thiophen-3-yl)propan-1-amine;
3,3-Dimethyl-1-(3-(methylamino)-1-(thiophen-2-yl)propyl)indoline-6-carbonitrile;
3-(3,3-Dimethylindolin-1-yl)-N-methyl-3-(thiophen-2-yl)propan-1-amine;
1-(3-(Methylamino)-1-(thiophen-2-yl)propyl)indoline-6-carbonitrile;
1-(3-(Methylamino)-1-(thiophen-2-yl)propyl)indoline-4-carbonitrile;
1-(3-(Methylamino)-1-(thiophen-2-yl)propyl)indoline-5-carbonitrile;
3,3-Dimethyl-1-(3-(methylamino)-1-(thiophen-2-yl)propyl)-2,3-dihydro-1H-pyrrolo[3,2-b]pyridine-6-carbonitrile;
N-methyl-3-(2-methylindolin-1-yl)-3-(thiophen-2-yl)propan-1-amine;
3-(5-Methoxy-3,3-dimethyl-2,3-dihydro-1H-pyrrolo[3,2-b]pyridin-1-yl)-N-methyl-3-(thiophen-2-yl)propan-1-amine;
3,3-Dimethyl-1-(3-(methylamino)-1-(thiophen-2-yl)propyl)indoline-4-carbonitrile;
1-(3-(Ethylamino)-1-(thiophen-2-yl)propyl)-3,3-dimethyl-2,3-dihydro-1H-pyrrolo[3,2-b]pyridine-6-carbonitrile;
(S)—N-methyl-3-(6-methyl-2,3-dihydro-1H-pyrrolo[3,2-b]pyridin-1-yl)-3-(thiophen-3-yl)propan-1-amine;
(R)—N-methyl-3-(6-methyl-2,3-dihydro-1H-pyrrolo[3,2-b]pyridin-1-yl-3-(thiophen-3-yl)propan-1-amine;
3,3-Dimethyl-1-(3-(methylamino)-1-(thiophen-3-yl)propyl)-2,3-dihydro-1H-pyrrolo[3,2-b]pyridine-5-carbonitrile hydrochloride;
(S)-3-(6-Fluoro-3,3-dimethyl-2,3-dihydro-1H-pyrrol[3,2-b]pyridin-1-yl)-N-methyl-3-(thiophen-2-yl)propan-1-amine;
(R)-3-(6-fluoro-3,3-dimethyl-2,3-dihydro-1H-pyrrolo[3,2-b]pyridin-1-yl)-N-methyl-3-(thiophen-2-yl)propan-1-amine;
(S)-3-(3,3-Dimethyl-5-(trifluoromethyl)-2,3-dihydro-1H-pyrrolo[3,2-b]pyridin-1-yl)-N-methyl-3-(thiophen-3-yl)propan-1-amine;
(R)-3-(3,3-dimethyl-5-(trifluoromethyl)-2,3-dihydro-H-pyrrolo[3,2-b]pyridin-1-yl)-N-methyl-3-(thiophen-3-yl)propan-1-amine;
(S)-3-(6-Chloro-3,3-dimethyl-2,3-dihydro-1H-pyrrolo[3,2-b]pyridin-1-yl)-N-methyl-3-(thiophen-2-yl)propan-1-amine;
(R)-3-(6-chloro-3,3-dimethyl-2,3-dihydro-1H-pyrrolo[3,2-b]pyridin-1-yl)-N-methyl-3-(thiophen-2-yl)propan-1-amine;
(S)-1-(3-(Methylamino)-1-(thiophen-2-yl)propyl)indoline-4-carbonitrile;
(S)-1-(3-(methylamino)-1-(thiophen-2-yl)propyl)indoline-4-carbonitrile;
(S)-3-(5-Methoxy-3,3-dimethyl-2,3-dihydro-1H-pyrrolo[3,2-b]pyridin-1-yl)-N-methyl-3-(thiophen-2-yl)propan-1-amine;
(R)-3-(5-methoxy-3,3-dimethyl-2,3-dihydro-1H-pyrrolo[3,2-b]pyridin-1-yl)-N-methyl-3-(thiophen-2-yl)propan-1-amine;
(S)-3,3-Dimethyl-1-(3-(methylamino)-1-(thiophen-2-yl)propyl)-2,3-dihydro-1H-pyrrolo[3,2-b]pyridine-6-carbonitrile;
(R)-3,3-dimethyl-1-(3-(methylamino)-1-(thiophen-2-yl)propyl)-2,3-dihydro-1H-pyrrolo[3,2-b]pyridine-6-carbonitrile;
(S)—N-methyl-3-((R)-2-methylindolin-1-yl)-3-(thiophen-2-yl)propan-1-amine;
(R)—N-methyl-3-((S)-2-methylindolin-1-yl)-3-(thiophen-2-yl)propan-1-amine;
(S/R)—N-methyl-3-((S/R)-2-methylindolin-1-yl)-3-(thiophen-2-yl)propan-1-amine;
(S)-1-(3-(Ethylamino)-1-(thiophen-2-yl)propyl)-3,3-dimethyl-2,3-dihydro-1H-pyrrolo[3,2-b]pyridine-6-carbonitrile and
(R)-1-(3-(ethylamino)-1-(thiophen-2-yl)propyl)-3,3-dimethyl-2,3-dihydro-1H-pyrrolo[3,2-b]pyridine-6-carbonitrile.

29. A process for the preparation of a compound of general formula (I) according to claim 17: 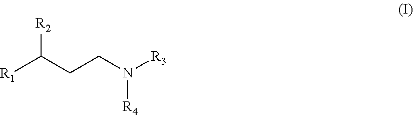
comprising:
a) a reduction reaction of a carboxamido compound of formula (IV): 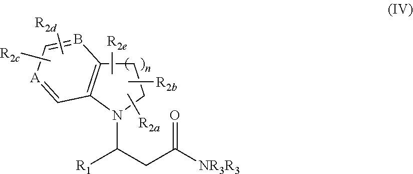
wherein R₁, R₂ₐ, R_{2b}, R_{2c}, R_{2d}, R₂ₑ, R₃, R₄, A, B and n are as defined in claim 17, or
b) reaction of a compound of formula (VI-H) or (VI-G): 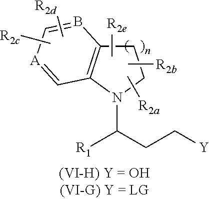
with a compound of formula (V):
HNR₃R₄ (v)
wherein R₁, R₂ₐ, R_{2b}, R_{2c}, R_{2d}, R₂ₑ, R₃, R₄, A, B and n are as defined in claim 17, and LG represents a suitable leaving group or
c) reaction of a compound of formula (II) 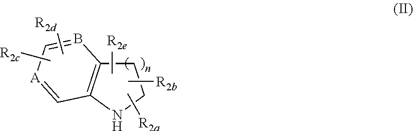
with a compound of formula (IIIc): 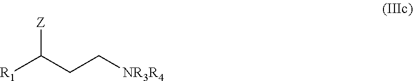
wherein R₁, R₂ₐ, R_{2b}, R_{2c}, R₂ₑ, R_{2d}, R₂ₑ, R₃, R₄, A, B and n are as defined in claim 17, and Z independently represents a leaving group or a hydroxy group.

30. A method for the treatment and/or prophylaxis of diseases and/or disorders mediated by the subunit α2δ, including α2δ-1 subunit of voltage-gated calcium channels and/or noradrenaline transporter (NET), in a subject in need thereof, comprising administration of an effective amount of the compound according to claim 17.

31. The method according to claim 30, wherein the disease or disorder is selected from the group consisting of pain, depression, anxiety and attention-deficit-/hyperactivity disorder (ADHD).

32. The method according to claim 31, wherein the pain is selected from the group consisting of neuropathic pain, inflammatory pain, chronic pain and other pain conditions involving allodynia and hyperalgesia.

33. A pharmaceutical composition comprising the compound according to claim 17, or a pharmaceutically acceptable salt, co-crystal, isomer, prodrug or solvate thereof, and at least a pharmaceutically acceptable carrier, additive, adjuvant or vehicle.
